(19) United States
(12) Patent Application Publication
(10) Pub. No.: US 2011/0081377 A1
(43) Pub. Date: Apr. 7, 2011

(54) AEROSOLIZED GENETIC VACCINES AND METHODS OF USE

(75) Inventors: Mario Roederer, Bethesda, MD (US); Srinivas Rao, Columbia, MD (US); Gary J. Nabel, Washington, DC (US); Charla A. Andrews, Washington, DC (US)

(73) Assignee: The United States, as represented by the Secretary Department of Health and Human Services, Bethesda, MD (US)

(21) Appl. No.: 12/887,286

(22) Filed: Sep. 21, 2010

Related U.S. Application Data
(63) Continuation-in-part of application No. PCT/US2009/001754, filed on Mar. 20, 2009.
(60) Provisional application No. 61/038,534, filed on Mar. 21, 2008.

(51) Int. Cl. A61K 39/295; A61K 39/00; A61K 39/12; A61K 39/02; A61K 9/14; A61K 39/235; A61P 37/04; A61P 31/12; A61P 31/04; A61P 31/10

(52) U.S. Cl. 424/202.1; 424/184.1; 424/204.1; 424/234.1; 424/274.1; 424/489; 424/233.1

(57) ABSTRACT

The present invention generally features methods for the mucosal delivery of immunogenic compositions and methods for treating or preventing diseases featuring such immunogenic compositions. In particular embodiments, the immunogenic compositions are genetic vaccines formulated as aerosols.

### CROSS-REFERENCE TO RELATED APPLICATION

This application is a continuation-in-part of PCT/US2009/001754, filed Mar. 20, 2009, which claims the benefit of U.S. Provisional Application No. 61/038,534, filed on Mar. 21, 2008, both of which are incorporated herein by reference in their entirety.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. This research was supported by the intramural program of the National Institutes of Allergy and Infectious Diseases, NIH. The Government may have certain rights in this invention.

### BACKGROUND OF THE INVENTION

Currently, many vaccines are administered by needle-and-syringe. Growing evidence suggests that mucosal immunization may offer operational advantages over intramuscular (IM) administration. Needle-free delivery methods may improve compliance, reduce discomfort, and improve safety of vaccines. Particularly in the developing world, needle-free delivery could mitigate the risk of blood-borne pathogen transmission by unsafe injection practices or inadequately sterilized equipment, and be easier and safer to deploy by non-medical personnel. There is growing interest in the use of needle-free methods of mucosal vaccine delivery, which could provide an alternative, cost-effective strategy to control diseases in, developing countries. Furthermore, methods for mucosal vaccine delivery directly to the lung would likely be advantageous for the treatment or prevention infections caused by respiratory pathogens, including tuberculosis, respiratory syncitial virus infection, and influenza.

Vaccine delivery to mucosal sites is typically achieved through introduction of the vaccine by intranasal or oral routes. But these forms of administration may be problematic. Oral administration of some viral vector vaccines has not elicited potent immune responses. Intranasal delivery of viral based vaccines has raised safety concerns because following intranasal delivery virus was found in the olfactory bulb of immunized animals. This finding raised concern that intranasally delivered recombinant viral based vaccines could disseminate to the central nervous system. Accordingly, improved methods of mucosal vaccine delivery are required. Such methods are of particular interest for the delivery of viral vector vaccines.

### SUMMARY OF THE INVENTION

As described below, the present invention features methods for the mucosal delivery of immunogenic compositions and methods for treating or preventing diseases featuring such immunogenic compositions.

In one aspect, the invention generally features a method of inducing a pathogen-specific immune response in a subject (e.g., a human). The method involves administering to the subject an aerosol genetic vaccine containing an effective amount of an expression vector containing a polynucleotide encoding a pathogen polypeptide and a pharmaceutically acceptable excipient.

In a related aspect, the invention features a method of treating or preventing a pathogen infection in a subject. The method involves administering to the subject an aerosol genetic vaccine containing an effective amount of an expression vector containing a polynucleotide encoding a pathogen polypeptide and a pharmaceutically acceptable excipient.

In another related aspect, the invention features a method of inducing a cellular immune response in a subject's lung. The method involves administering to the subject an aerosol genetic vaccine containing an effective amount of an expression vector containing a polynucleotide encoding a pathogen polypeptide and a pharmaceutically acceptable excipient. In one embodiment, the method further induces a humoral immune response in lung, peripheral blood, and mucosal sites.

In yet another related aspect, the invention features a method of inducing a systemic humoral response in a subject. The method involves administering to the subject an aerosol genetic vaccine containing an effective amount of an expression vector containing a polynucleotide encoding a pathogen polypeptide and a pharmaceutically acceptable excipient.

In still another related aspect, the invention features a method of inducing a pathogen-specific humoral and/or cellular immune response in a subject having pre-existing anti-adenoviral immunity. The method involves administering to the subject an aerosol genetic vaccine containing an effective amount of an adenoviral expression vector containing a polynucleotide encoding a pathogen polypeptide and a pharmaceutically acceptable excipient.

In another aspect, the invention features a method of selectively administering to the upper airway of a subject an aerosol genetic vaccine containing an effective amount of an expression vector containing a polynucleotide encoding a pathogen polypeptide and a pharmaceutically acceptable excipient. In one embodiment, the particles of the vaccine have a mass median diameters of about 10-12 μm (e.g., 10, 10.5, 11, 11.4, 12 μm).

In another aspect, the invention features a method of administering to the upper total airway epithelia of a subject an aerosol genetic vaccine containing an effective amount of an expression vector containing a polynucleotide encoding a pathogen polypeptide and a pharmaceutically acceptable excipient. In one embodiment, the particles of the vaccine have mass median diameters of about 3-5 μm (e.g., 3, 3.5, 4, 4.4, 5 μm). In one embodiment, of the preceding aspects, the method delivers between about 10⁹ and 10¹¹ particles (e.g., 10⁹, 10¹⁰, 10¹¹).

In another aspect, the invention features a method for inducing protective immunity in a subject (e.g., human) against tuberculosis. The method involves administering to the subject an aerosol genetic vaccine containing an adenoviral vector containing a polynucleotide encoding tuberculosis antigens 85A/B or 10.4.

In another aspect, the invention features an immunogenic composition containing an effective amount of an expression vector containing a polynucleotide encoding a pathogen polypeptide and a pharmaceutically acceptable excipient, where the immunogenic composition is formulated for aerosol delivery to the lung. In various embodiments, the expression vector is any one of an adenoviral vector, lentiviral vector, adeno-associated viral vector, retroviral vector, lenti viral vector, human parainfluenza virus type 1 vector, human coronavirus 229E vector, paramxyoviral vector (PV), respiratory syncytial viral vector (RSV), or human parainfluenza virus (PIV)-based vector.

In another aspect, the invention features an immunogenic composition containing an adenoviral expression vector containing a pathogen polynucleotide encoding a polypeptide that is any one of TB 85A/B or 10.4 antigens, influenza hemagglutinin or nucleoprotein, HIV or SIV Gag-Pol and HIV or SIV Env, where the pathogen polynucleotide is positioned for expression in a mammalian cell and a pharmaceutically acceptable excipient, where the immunogenic composition is formulated for aerosol delivery to lung tissue.

In yet another aspect, the invention features a genetic vaccine in an amount sufficient to induce a protective immune response in a subject, the vaccine containing an adenoviral expression vector containing a pathogen polynucleotide encoding a polypeptide that is any one of TB 85A/B or 10.4 antigens, influenza hemagglutinin or nucleoprotein, HIV or SIV Gag, Pol or Env, having a mass median diameter between about 1 and 15 μm and a pharmaceutically acceptable excipient, where the vaccine is formulated for aerosol delivery to the lung.

In still another aspect, the invention features a pharmaceutical pack containing an effective amount of an immunogenic composition of any previous aspect, a device for aerosol delivery of the composition, and written instructions for use of the pack for the treatment or prevention of a pathogen infection. In one embodiment, the device is a nebulizer or metered device inhaler. In another embodiment, the pack further comprises an adjuvant. In yet another embodiment, the composition comprises between about 10⁹ to 10¹¹ particles.

In still another aspect, the invention features a device for dispersing a genetic vaccine into particles and delivering a dose of the particles to a subject, the device containing an effective amount of an expression vector containing a polynucleotide encoding a pathogen polypeptide.

In another aspect, the invention features a device for dispersing a genetic vaccine into particles and delivering a dose of the particles to a subject, the device containing an immunogenic composition of any previous aspect or a composition delineated herein. In one embodiment, the device is a nebulizer (e.g., jet, ultrasonic, pressurized and vibrating porous plate nebulizer), metered dose inhaler or dry powder inhaler. In another embodiment, the composition comprises particles having a mass median diameter between about 1 and 15 μm. In another embodiment, the device selectively delivers the vaccine to total lung tissue.

In another aspect, the invention features a method for production of an aerosol genetic vaccine for the treatment or prevention of a pathogen infection. The method involves providing an effective amount of an immunogenic composition containing an effective amount of an expression vector containing a polynucleotide encoding a pathogen polypeptide and a pharmaceutically acceptable excipient; and dispersing the composition into particles having a median mass median diameter of between 1 and 15 μm.

In another aspect, the invention features a method for production of an aerosol genetic vaccine for the treatment or prevention of a pathogen infection. The method involves providing an effective amount of an immunogenic composition containing an effective amount of an expression vector containing a polynucleotide encoding a pathogen polypeptide and a pharmaceutically acceptable excipient; contacting the immunogenic composition with a vibrating membrane; and dispersing the composition into droplets having a median mass median diameter of between 1 and 15 μm.

In various embodiments of the above aspects, the expression vector is any one of an adenoviral vector, lentiviral vector, adeno-associated viral vector, retroviral vector, lentiviral vector, human parainfluenza virus type 1 vector, human coronavirus 229E vector, paramxyoviral vector (PV), respiratory syncytial viral vector (RSV), or human parainfluenza virus (PIV)-based vector. In other embodiments of the above aspects, the expression vector comprises a pathogen polynucleotide encoding a polypeptide that is any one of TB 85A/B or 10.4 antigens, influenza hemagglutinin (HA), HIV or SIV Gag-Pol and HIV or SIV Env, where the pathogen polypeptide is positioned for expression in a mammalian cell. In other embodiments of the above aspects, the expression vector is an adenoviral vector. In one embodiment, the composition comprises droplets having a mass median diameter of between about 1 and 15 μm, where the bottom limit of the range is any integer between 1 and 14 and the upper limit of the range is any integer between 2 and 15. Alternatively, the droplets/particles have a mass median diameter of about 3-5 μm or 9-12 μm. In another embodiment, the composition contains droplets having a mass median diameter of about 11.4 μm or 4.4 μm. In another embodiment, the dispersed composition comprises between about 10⁹ to 10¹¹ particles.

In other embodiments of the above aspects, a pathogen polypeptide is a bacterial (e.g., tuberculosis 10.4 or 85A/B antigens), viral (e.g., influenza, respiratory syncytial virus (RSV), SIV or HIV polypeptide), fungal, or yeast polypeptide. In other embodiments of the above aspects, the viral polypeptide is an Env, Gag, Pol (e.g., HIV or SIV Gag, Pol, Or Env), influenza hemagglutinin, or influenza nucleoprotein. In still other embodiments of the above aspects or any other aspect of the invention delineated herein, the expression vector is an adenoviral expression vector containing a pathogen polynucleotide encoding a polypeptide that is any one of TB 10.4 or 85A/B-specific antigens, influenza hemagglutinin (HA) or nucleoprotein, HIV or SIV Gag-Pol and HIV or SIV Env, or a fragment thereof. In one embodiment, the humoral response comprises IgA and IgG isotypes. In another embodiment, the method induces a protective immune response against a mucosal pathogen. In still another embodiment, the vaccine is administered to a subject at least about 2 and 20 times (2, 3, 4, 5 times), such as once a day, once a week, once a month or annually. In other embodiments of the above aspects, the method generates humoral, CD4 and CD8 T cell responses. Such as where the CD4 and CD8 T cells are polyfunctional making two or more of IFNγ, TNF, and IL2. In other embodiments of the above aspects, serum IgG levels peak at about 4, 6, or 8 weeks after immunization. In other embodiments of the above aspects, the immunity persisted for at least about 3, 6, 9 or 12 months. In other embodiments of the above aspects, the method generates a negligible anti-vector immune response. In other embodiments, the method induces an antibody response at mucosal surfaces. In other embodiments, the immunogenic composition is administered to a respiratory, pulmonary airway, gastrointestinal, genitourinary, or secretory gland mucosal surface. In other embodiments, the method induces a large T cell response in the lung of the subject. In other embodiments, the adenoviral vector (e.g., a replication-incompetent recombinant adenoviral vector) is rAd5 or rAd35. In other embodiments, the vaccine was administered as a single dose containing about 10⁹-10¹¹ particles. In other embodiments, the methods further involve administering a second dose. In other embodiments, the method induces 25-50% of CD8 T cells and from 3-12% of CD4 T cells. In other embodiments, the viral polypeptide is any one of an influenza, respiratory syncytial virus (RSV), SIV, HIV, herpes virus, lentivirus, adenovirus, rhinovirus, and rotavirus polypeptide. In other embodiments, the composition further comprises an adjuvant or a cytokine.

The invention provides immunogenic compositions formulated as aerosols and related therapeutic and prophylactic methods. Other features and advantages of the invention will be apparent from the detailed description, and from the claims.

### DEFINITIONS

As used in the specification and claims, the singular form “a”, “an” and “the” include plural references unless the context clearly dictates otherwise.

The term “aerosol” or “aerosolized” as used herein is meant to refer to dispersions in air of solid or liquid particles. In general, such particles have low settling velocities and relative airborne stability. In certain embodiments, the particle size distribution is between 0.01 um and 15 um.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a Marker (e.g., gene or polypeptide) as detected by standard art known methods, such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant any immunoglobulin polypeptide, or fragment thereof, having immunogen binding ability.

The term “cell mediated immune response” is meant to refer to an immune response that involves the activation of macrophages, natural killer cells, antigen-specific T-lymphocytes, or the release of various cytokines in response to an antigen. In certain preferred examples, the size of the immunogenic particles is such that the cell mediated immune response is activated in the absence of the activation of the humoral immune response. In particular examples, an immunogenic particle size of between 10-15 microns is sufficient to activate a cell mediated immune response in the absence of a humoral immune response.

“Detect” refers to identifying the presence, absence or amount of the analyte to be detected.

By “disease” is meant any condition or disorder that damages or interferes with the normal function of a cell, tissue, or organ. Examples of diseases include pathogen infections, such as a viral, bacterial, yeast, or fungal infection, invasion or colonization of a host cell.

By “effective amount” is meant the amount of an agent required to prevent or ameliorate the symptoms of a disease relative to an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “genetic vaccine” is meant an immunogenic composition comprising a polynucleotide encoding an antigen.”

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The term “humoral immune response” is meant to refer to the aspect of the immune response that is mediated by secreted antibodies or by B lymphocytes.

By “immunogenic composition” is meant a composition capable of inducing an immune response when introduced to a subject.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the type of cells involved. Immune responses include cell-mediated responses, humoral responses, or innate responses.

The term “in combination” as used herein refers to the use of more than one therapy. The use of the term “in combination” does not restrict the order in which therapies are administered. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 or more weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 or more weeks after) the administration of a second therapy. The invention contemplates using administration of a genetic vaccine in combination with a prime boost, an adjuvant, an additional conventional or genetic vaccine, and/or additional prophylactic and/or therapeutic agents (e.g., antiviral agents, antibacterial agents, anti-fungals, or any other antibiotic or biocide).

By “immunomodulatory protein” is meant any protein that alters an immune response. An alteration in the immune response is an increase or decrease in the response.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

The term “mucosal surface” is meant to refer to any epithelial membrane lining a body cavity or passage. Mucosal surfaces include, but are not limited to, the respiratory tracts, gastrointestinal tracts, genitourinary tracts, and secretory glands. In certain embodiments, a mucosal surface is a distal mucosal surface, for example a mucosal surface located at a point that is distant from the point of administration of the immunogenic composition. For example, in certain embodiments, the immunogenic composition is administered to the respiratory tract, and the immune response is generated at a distal mucosal surface. Distal mucosal surfaces in certain examples, include, but are not limited to, the rectal or vaginal mucosa.

The term “nebulizer” as used herein is meant to refer to any device that disperses an agent as an aerosol. In certain examples, the device generates an aerosol comprising particles that are between about 0.01-15 microns in size. In preferred examples, when a vaccine formulation or immunogenic composition is applied to the device, the resulting aerosol contains the vaccine and can deliver it into the lungs of a subject by normal breathing.

As used herein, “obtaining” as in “obtaining an agent” includes synthesizing, purchasing, or otherwise acquiring the agent.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

The terms “particles,” “aerosolized particles,” or “droplets” are used interchangeably to refer to immunogenic agents dispersed as an aerosol. In one embodiment, the particles are formed by dispersing a vaccine using a vibrating membrane. In another embodiment, the particles are formed upon forcing the composition through a flexible porous membrane or by dispersing the composition using a propellant. The size of the particles allows them to remain suspended in air for a sufficient amount of time such that they can be administered to a subject's lung or other mucosal surface. Preferably, the particles have a size in the range of 0.01 microns to about 15 microns, in some embodiments about 2 to about 5 microns, in other embodiments about 10 to about 15 microns.

By “pathogen” is meant any bacteria, virus, or fungi, capable of interfering with the normal function of a cell.

By “pathogen-specific immune response” is meant an immune response that targets a pathogen antigen to a greater degree than other antigens.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “protective immune response” is meant an immune response that reduces a subject's propensity to develop a pathogen infection, that reduces the severity of the infection, or that ameliorates a symptom of a pathogen infection.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

As used herein, the term “respiratory virus” refers to a virus that infects a cell of the respiratory tract. The respiratory tract includes, for example, air passages from the nose to the pulmonary alveoli, through the pharynx, larynx, trachea, and bronchi.

By “selectively administer” is meant administer to a desired target tissue or organ to a greater extent than to other tissues or organs. For example, at least about 55%, 60%, 70%, 80%, 90% or more of the agent is administered to the upper airways, and less than about 50%, 40%, 30%, 20%, 10%, or 5% is administered to other tissues or organs.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a bovine, equine, canine, ovine, rodent, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A-1H show cellular immune responses induced by aerosolized immunization with rAd5 encoding an antigen. FIG. 1A includes two graphs showing spot forming cells (SFC) per 10⁶ cells plotted as a function of time. The terms env, pol and gag refer to the SIV inserts present in the rAd5 adenoviral vector (Letvin J Virol 78 (14), 7490, 2004) particles encoding an SIV Gag-Pol fusion protein. Following immunization with rAd5 encoding antigen at week 0 and week 3, T cell responses were measured by IFNγ ELISpot to SIV Gag-Pol and Env peptides in peripheral blood monocytes (PBMC). FIG. 1B includes two graphs showing the percentage of T cells responding to Env, Gag, or Pol as a function of time. Robust and persistent CD4 and CD8 T cell responses were measured in the Bronchoalveolar lavage (BAL) by intracellular cytokine staining (ICS); the responses to SIV Gag-Pol and Env peptides are summed. FIG. 1C provides plots displaying flow cytometry data showing an example of the cytokine responses in BAL CD4 and CD8 T cells for one animal (4 μm aerosol) at 55 weeks, one year after the immunization. The flow cytometric gating strategy is shown (top). FIGS. 1D-1F show that dose titration of rAd5 encoding an antigen showed little effect on the total BAL T cell magnitude (FIG. 1D) when measured 8 weeks after a single immunization. FIG. 1E shows the quality of the T cell response induced by various doses of rAd5 encoding an antigen. The fraction of the response that is polyfunctional (3+) or solely IFNγ is highlighted by arrows. FIG. 1F provides six pie charts. The quality is represented as pie charts depicting the polyfunctionality of the response: antigen-specific cells secreting a single cytokine are depicted in light grey (1+), two cytokines in dark gray (2+), and all three cytokines in black (3+). FIG. 1G provides two graphs showing the percentage of BAL CD8 and CD4 following stimulation by rAd5 with an SIV insert or rAd5 with HIV env insert. Three animals were immunized with 10¹¹ particles of rAd5 encoding SIV Env and Gag-Pol by aerosol (black at left) or aerosol and IM simultaneously (grey); de novo responses to the SIV proteins are shown (left). Neutralizing titres in the serum following this first immunization are shown in Table 1. These six animals were then immunized with 10¹⁰ particles of aerosolized rAd5 encoding HIV EnvA (middle); shown are the memory responses to either SIV proteins (left) or the de novo responses to HIV proteins (right). For comparison, the de novo HIV responses in three different animals that received only aerosolized HIV Env rAd5 are shown (right). FIG. 1H provides two graphs showing the percentage of BAL CD8 and CD4 following stimulation by rAd5 with an SIV Env insert. Ten animals were immunized with 5×10⁹ particles of rAd5 encoding SIV Env. Seven of these animals were seronegative for rAd5 at the time of immunization (black at left), and three were serpositive from naturally acquired infection (grey at right).

FIGS. 2A-2G are graphs showing humoral responses induced by immunization with aerosolized rAd5 encoding an antigen. FIGS. 2A and 2B show the induction of IgG and IgA, respectively, to the vector inserts, SIV Env (left) and SIV Gag-Pol (right). IgG and IgA were measured in serum. Data is shown as specific activity, i.e., μg of antigen-specific antibody of a given isotype per mg of total antibody of that isotype for each animal (n=3 per group). The empty inverted triangles represent one animal that had high pre-immunization anti-gag-pol cross-reactivity. FIGS. 2C and 2D show the rAd5 dose-response induction of HIV EnvA-specific IgG and IgA, respectively, in serum and three mucosal sites. FIGS. 2E and 2F show the induction of HIV EnvA specific IgG and IgA, respectively, at week 6 for the animals described in FIG. 1G. Prior Ad5 exposure occurred 6 months (aerosol+IM) or 14 months (aerosol) before the aerosolized rAd5 HIV-env administration. FIG. 2G includes eight graphs showing humoral responses induced by immunization with aerosolized rAd5 encoding the indicated antigen in Ad-seropositive animals. The full kinetics for all six animals referred to in FIG. 1G and FIGS. 2E and 2F are shown.

FIGS. 3A-C are graphs comparing intranasal and aerosol delivery of rAd, while FIGS. 3D and 3E depict graphs and pie charts, respectively, showing cellular immunity induced by immunization with aerosolized rAd35 encoding the indicated antigen. With respect to FIGS. 3A-C, 10¹⁰ particles of rAd 5 encoding HIV EnvA were given to eight female animals at weeks 0 and 4 by the aerosol route (grey at right, n=4) or intranasally (black at left, n=3). FIG. 3A shows the total CD4 and CD8 response (i.e. cells making IL2, TNF, or IFNγ after ex vivo stimulation) at the peak of response (week 7) for BAL (left panel) and PBMC (right panel). FIGS. 3B and 3C show the HIV Env-specific levels of IgG and IgA, respectively, assayed in serum and three different mucosal tissues four weeks after immunization by either intranasal (black) or aerosol (grey) delivery. FIG. 3D shows TB antigens 85A/B-specific BAL responses for CD4 and CD8 T cells measured by intracellular cytokine staining as described in FIG. 1B at the indicated time points post-immunization. rAd35 encoding the TB antigens 85A, 85B, and 10.4 was given by 4 μm aerosols to four animals at three different doses, or to three animals at 10¹¹ particles by intramuscular injection. Animals were immunized at week 0 and week 8. Shown are the 85A/B-specific BAL responses for CD4 and CD8 T cells measured by intracellular cytokine staining as in FIG. 1B at the indicated time points post-immunization. FIG. 3E shows the quality of the CD4 and CD8 T cell responses in the BAL to 85A/B peptides (week 17) as shown for rAd5 encoding an antigen in FIG. 1E.

FIGS. 4A-4I are graphs showing that immunization with aerosolized rAd5 encoding an antigen provides protective immunity to live viral challenge. Four groups of ferrets (n=5) were immunized using either an intramuscular (IM) rAd5 encoding influenza hemagglutinin (HA), or aerosolized (AE) sham rAd5, rAd5 with a HA, or nucleoprotein (NP) rAd5. 18 days later, animals were challenged with a lethal dose of H5N1. FIG. 4A shows nasal viral load as a function of time for the indicated adenoviral vectors and inserts. Viral load in the nasal wash immediately post-challenge shows early control by the HA-immunized animals. Lines indicate the mean±1 sd. FIG. 4B is a survival plot showing that the HA-immunized animals were completely protected against the lethal challenge. FIG. 4C shows the percentage of neutralizing antibodies present in animals immunized with hemagglutinin (HA), nucleoprotein (NP) or sham immunized using intramuscular (IM) or aerosol (AE) delivery. Neutralizing antibodies to the immunization strain were detectable only in the sera of animals immunized by IM (and one animal immunized by aerosol). FIG. 4D shows that no neutralizing antibodies to the challenge strain were detected. For FIGS. 4E-I, three groups of ferrets (n=15 each) were immunized using either a sham rAd5 (aerosol delivery) or a rAd5 encoding influenza HA (Indonesia) by either IM or aerosol delivery. After 18 days, 5 animals in each group were euthanized to measure cellular responses, while the remaining 10 animals were challenged with a lethal dose of H5N1 (Vietnam). FIG. 4E shows that protection in vaccinated groups was 100%. FIG. 4F shows a time course that plots relative weight in comparison to weight a day 0. FIG. 4G shows viral loads as measured in the three groups at 2, 4, and 7 days post-infection. FIG. 4H depicts the induction of IFNγ mRNA in BAL following restimulation with HA peptides ex vivo (each bar represents one animal measured in triplicate, and the error bar represents±1 standard deviation). FIG. 41 shows neutralization of Indonesia H5N1 vaccine strain by sera from all fifteen animals at the time of challenge. A single animal in the aerosol group (light grey solo dots at bottom of plot) showed no neutralization, as well as higher nasal viral loads and significant weight loss, but met no other criteria for euthanasia prior to the termination of the experiment.

FIG. 5 shows the investigational eFlow™ Nebulizer System components and operation. The device produces a dense aerosol from a vibrating metal membrane nebulizer. The reagent is poured into a medication reservoir and the membrane is activated. The dense aerosol is “mist-like,” and is inhaled during the receiver's respiration. The elapsed time of membrane activation is related to amount and viscosity of reagent. Variable volumes can be used from 0.5 to 4 mL. Particle size can vary depending on the aerosol head size used. In the experiments described herein, aerosol heads were used that generated particles of MMD=4.4±1.6 μm or 11.4±1.8 μm in size, delivering a 1 mL volume over a 1 minute time frame.

FIGS. 6A-6C are graphs showing clinical pathology evaluations for white blood cell counts, lymphocyte percentages, alanine aminotransferase (ALT) level, aspartate aminotransferase (AST) level, blood urea nitrogen level, and creatinine. In addition to the daily clinical observations post-immunization, all animals were periodically evaluated for clinical pathology parameters including complete blood count (CBC) and clinical chemistries. Shown are clinical values for (a) complete blood count (CBC), (b) liver function tests, and (c) kidney function tests for macaques immunized as described in FIG. 1A (10¹¹ particles of rAd5 encoding an antigen): red lines are the averages for 3 animals immunized with 4 μm aerosol; blue are averages for 3 animals immunized with 11 μm aerosol. Dotted lines represent the normal ranges. All parameters were generally within normal ranges, with no specific trends observed concomitant with the immunizations performed at weeks 0 and 3.

FIG. 7 includes three micrographs showing the histology of lung tissue after aerosolized immunization. Representative sections of different lobes of macaque lungs, bronchial lymph node and tonsils were collected as discussed in the Methods sections. Two animals were immunized with 10¹¹ rAd5-HIV and euthanized either 2 or 9 days later. Hematoxylin and eosin stained samples at 100X of the representative lung section are shown. Interstitial congestion and intra-alveolar eosinophilic material in immunized animals represents proteinaceous fluid from recent bronchial alveolar lavage. In general, the lungs appeared to be within normal limits.

FIG. 8 provides the sequence of Ad5-SIV gag-pol.

FIGS. 9A and 9B provides the sequences of the SIV gag-pol and env inserts.

FIG. 10 provides a map of an exemplary E1/E3 deleted rAd35 vector (Vogels, 2008/0199917).

### DETAILED DESCRIPTION OF THE INVENTION

The invention features immunogenic compositions and methods for inducing a therapeutic or prophylactic immune response featuring such compositions. The invention is based, at least in part, on the discovery that aerosal delivery of replication-incompetent recombinant adenoviral vectors expressing gene products from infectious pathogens targeted the entire lung surface of nonhuman primates that inhaled the vaccine through normal respiration. In nonhuman primates, this regimen induced remarkably high, and stable, lung T cell responses, as well as systemic and mucosal humoral responses of both IgA and IgG isotypes. Moreover, strong immunogenicity was achieved even in the face of pre-existing anti-adenoviral immunity—overcoming a critical hurdle to the use of these vectors in humans. In a lethal challenge model, this immunization route completely protected ferrets against H5N1 highly pathogenic avian influenza virus. Taken together, the unique immunogenicity profile offered by this route of immunization offers a powerful approach to generating protective immune responses against mucosal pathogens. Because it elicits both systemic and mucosal humoral immunity, this approach may be applicable to sexually transmitted pathogens, such as HIV. Importantly, this vaccination approach can be used repeatedly to generate humoral, CD4, and CD8 T cell responses. Accordingly, the invention provides aerosol immunogenic compositions for use in generating an immune response in a subject, as well as therapeutic and prophylactic methods employing such immunogenic compositions for the treatment or prevention of disease, such as pathogen infections.

### Mucosal Immunization

The generation of potent cellular and humoral responses at mucosal surfaces has been a goal of immune intervention against a variety of disease, including tuberculosis, influenza, respiratory syncytial virus (RSV), SIV and HIV. In the case of tuberculosis, T cell responses to primary exposure in the lung are not detectable for several weeks; by contrast, in the setting of Bacillus of Calmette and Guérin (BCG) vaccination, lung T cell responses can be observed within 14 days of infection. However, while BCG vaccination provides proven efficacy against systemic infection, it provides only variable protection against pulmonary infection. Without wishing to be bound by theory, having a high frequency of T cells resident in the lung at the time of tuberculosis infection may be important for optimal protection against pulmonary infection. Protection against RSV disease may similarly require potent Th1-biased or CD8⁺ T cell responses in the respiratory tract at the time of infection. In the case of HIV, there is substantial evidence that effective cytotoxic T cell responses in the gut mucosa during acute HIV infection can ameliorate pathogenesis. Hence, vaccination at mucosal sites offers a direct way to generate such responses.

Respiratory delivery may be optimal for targeting pathogens that infect respiratory tissues, such as tuberculosis, RSV, and influenza. Safety is an important concern when considering eliciting immune responses in the lung. To date, no animal studies have revealed safety concerns arising from delivery of immunogens by aerosol. As reported in more detail below, the immunogenicity of replication-defective recombinant adenoviruses encoding HIV, SIV, tuberculosis, and influenza genes was tested when the viruses were delivered as a fine aerosol into the lung. In nonhuman primates, this regimen induced very high, stable cellular immune responses that were localized to the lung, as well as humoral responses in the lung, peripheral blood, and, importantly, at multiple mucosal sites. In ferrets, this regimen induced protective immunity against H5N1 (pandemic) influenza challenge. These data indicate that aerosolized adenoviral vectors are highly useful for vaccination against respiratory infections, such as tuberculosis, influenza, and RSV, and provide a platform for generating mucosal antibody responses against other pathogens.

### Expression Vectors for Use in the Invention

The invention provides immunogenic compositions for delivery as aerosols having droplets between about 1 and 15 μm in size. The immunogenic composition comprises an expression vector suitable for expression in a mammalian cell, where the expression vector comprises a polynucleotide encoding a pathogen polypeptide. In one example, the expression vector is an adenoviral vector. Adenoviral vectors transduce a wide range of target cells resulting in high-level gene expression. Ad5 vectors are the most commonly used adenovirus vector. Ad5 vectors transduce a wide range of cell types. Ad5 vectors utilize the Coxsackie-Adenovirus Receptor (CAR) to enter cells. Transduction efficiency is related to the level of CAR expression on the cell membrane. Ad5 expression vectors useful for expressing a polypeptide in a mammalian (e.g., human) cell are known in the art and are described herein.

For example, U.S. Patent Publication Kovesdi et al., 20020110545, entitled “Adenovector pharmaceutical composition,” incorporated by reference in its entirety, and Nabel et al., 20070207166, entitled “Method of Using Adenoviral Vectors to Induce an Immune Response,” incorporated by reference in its entirety, describe adenoviral vectors, including replication deficient Ad5 expression vectors, useful in the compositions and methods of the present invention. Other descriptions of Ad5 vectors are provided, for example, by WO 00/03029, WO 02/24730, WO 00/70071, and WO 02/40665. Recombinant Ad5 expression vectors are commercially available, for example from GenVec.

Given that the Ad5 genome has been completely sequenced, particular embodiments of the present invention are described with respect to the Ad5 serotype. Any subtype, mixture of subtypes, or chimeric adenovirus may be used as the source of DNA for generation of a replication deficient adenoviral vector. In one embodiment, an adenoviral vector useful in the compositions or methods of the invention is at least deficient in a function provided by early region 1 (E1) and/or one or more functions encoded by early region 2 (E2), such as early region 2A (E2A) and early region 2B (E2B), and/or early region 3 (E3), and/or early region 4 (E4) of the adenoviral genome. Any one of the deleted functional regions may be replaced with a promoter-variable expression cassette to produce a novel gene product. The insertion of a novel gene into the E2A region, for example, may be facilitated by the introduction of a unique restriction site, such that the novel gene product may be expressed from the E2A promoter.

In addition, complementing cell lines for propagation of multiply deficient adenoviral vectors are known in the art. Such cell lines are known in the art and described, for example, in U.S. Patent Publication Kovesdi et al., 20020110545. In one embodiment, a cell line is characterized in complementing for at least one gene function of the gene functions comprising the E1, E2, E3 and E4 regions of the adenoviral genome. Other cell lines include those that complement adenoviral vectors that are deficient in at least one gene function from the gene functions comprising the late regions, those that complement for a combination of early and late gene functions, and those that complement for all adenoviral functions. One of ordinary skill in the art will appreciate that the cell line of choice would be one that specifically complements for those functions that are missing from the recombinant multiply deficient adenoviral vector of interest and that can be generated using standard molecular biological techniques. The cell lines are further characterized in containing the complementing genes in a non-overlapping fashion, which eliminates the possibility of the vector genome recombining with the cellular DNA. Accordingly, replication-competent adenoviruses are eliminated from the vector stocks, which are, therefore, suitable for certain therapeutic purposes, especially gene therapy purposes. This also eliminates the replication of the adenoviruses in noncomplementing cells.

Ad35 vectors are known in the art and are described, for example, by Vogels et al., U.S. Patent Publication No. 20080199917, entitled “Means and methods for producing adenovirus vectors,” and by Pau et al., U.S. Patent Publication No. 20070088156, entitled “Recombinant viral-based malaria vaccines,” each of which is incorporated herein by reference. Recombinant Ad35 expression vectors are commercially available, for example, from Crucell Holland B. V., which provided the replication-deficient adenovirus 35 vector used in the experiments described herein. Aeras 402 expresses M. tuberculosis antigens Ag85A, Ag85B, and TB 10.4. AERAS-402, and is available from Crucell Holland B. V. and the Aeras Global TB Vaccine Foundation. Radosevic et al., Infect Immun 75 (8), 4105 (2007) describe methods for generating the rAd vector serotype 35, which expresses a unique fusion protein of Mycobacterium tuberculosis (Mtb) antigens Ag85A, Ag85B and TB 10.4 (Ad35-28 TBS), described in Example 4.

Briefly, Radosevic states that PER.C6® cells were co-transfected with an adaptor pAdApt35Bsu plasmid containing TBS insert in the former E1 region and plasmids containing the remaining viral genome, all pre-digested with appropriate enzymes to liberate the Ad sequences from the plasmid backbone. Viruses were plaque purified and propagated on adherent-cell cultures. Purified stocks were obtained using standard CsCl gradient centrifugation, and upon dialysis steps formulated in PBS/5% sucrose (Radosevic, Materials and Methods, recombinant Ad35 TBS).

Methods for making the recombinant Ad35 vector described by Radosevic supra are described by Havenga et al., J Gen Virol. 2006 August; 87(Pt 8):2135-43, which is hereby incorporated by reference in its entirety. Briefly, Havenga teaches that to generate an rAd35 genome carrying Ad5-derived E4-Orf6 sequences, the Ad5 E4-Orf6 sequence was first cloned into a pBr plasmid containing Ad35 sequences (nt 18138 to right ITR: GenBank accession no. AY271307). This plasmid, called pBr.Ad35PRN, served as a template to amplify an Ad35 genome fragment of 1·8 kb (nt 30099-31880, containing a 39 tail homologous to Ad5 sequence) by using primers E4-F1 (59-AGAGGAACACATTCCCCC-39) and E4-R2 (59-GGGGAGAAAGGACTGTGTATTCTGTCAAATGG-39). A second PCR was performed, using as template a cosmid clone containing Ad5 sequences from nt 3534 to the right ITR (pWE.Ad5.AflII-rITR; Vogels et al., 2003; Holterman et al., 2004) and primers E4-F3 (59-TTTGACAGAATACACAGTCCTTTCTCCCCGGCTGG-39) and E4-R4 (59-ACAAAATACGAGAATGACTACGTCCGGCGTTCC-39). This amplification reaction resulted in a 1?1 kb fragment corresponding to Ad5 sequence nt 32963-34077 (numbering as in GenBank accession no. M73260) and flanked by sequences homologous to the first (39 end) and third (59 end) PCR fragments. A third, 357 by fragment corresponding to nt 32972-33329 in the Ad35 genome sequence and having a 59-homologous tail to the second PCR fragment was obtained by using pBr.Ad35PRN as template in combination with primers E4-F5 (59-GGACGTAGTCATTCTCGTATTTTGTATAGC-39) and E4-R6 (59-TCACCAACACAGTGGGGG-39). The three PCR fragments were combined via assembly PCR using the two outer primers (E4-F5 and E4-R2). The resulting 2·8 kb DNA fragment was digested with MluI and NdeI and cloned in an MluI-NdeI-digested plasmid called pBr.Ad35.PRNDE3. This gave rise to a plasmid called pBr.Ad35.PRNDE3.5Orf6. Plasmid pBr.Ad35.PRNDE3 is similar to pBr.Ad35.PRN, but carries a 2·6 kb deletion within the E3 region (corresponding to nt 27648-30320 of the Ad35 genome).

To create a new adaptor plasmid containing the pIX promoter, plasmid pAdapt35.IPI (Vogels et al., 2003) was modified. Hereto, first an AgeI restriction site located in the multiple cloning site behind the cytomegalovirus (CMV) promoter needed to be removed by partial AgeI restriction, followed by filling in protruding ends with Klenow enzyme and religation. This plasmid was subsequently digested with BglII, blunted with Klenow and further digested with AgeI. Ligation of the blunted AgeI DNA fragment to a Bsu36I-AgeI fragment corresponding to nt 3234-4251 in Ad35, in which the Bsu36I site was blunted with Klenow enzyme, resulted in the expected plasmid called pAdapt35.Bsu. To generate an Ad35.E1B.Luc vector, a pBr plasmid containing Ad35 genome sequence (left ITR to nt 4669) was used in which the E1A′sequences between SnaBI and HindIII (nt 452-1338 in Ad35) were replaced by an AvrII-BglII fragment (CMV-Luciferase-SV40 pA) derived from pAdapt35.Luc (Vogels et al., 2003). This plasmid thus contains the E1B promoter and coding sequence in its native position relative to pIX. The triple-antigen insert (TB-S), as well as single antigen-encoding DNAs, were obtained via gene synthesis of codon-optimized DNA sequences for expression in humans (Geneart GmbH) and contain the Mycobacterium tuberculosis antigens Ag85A (SwissProt accession no. P17944; aa 44-338), provided below:

| | |
|---|---|
| 1 | mqlvdrvrga vtgmsrrlvv gavgaalvsg lvgavggtat agafsrpglp veylqvpsps |
| 61 | mgrdikvqfq sgganspaly lldglraqdd fsgwdintpa fewydqsgls vvmpvggqss |
| 121 | fysdwyqpac gkagcqtykw etfltselpg wlqanrhvkp tgsavvglsm aassaltlai |
| 181 | yhpqqfvyag amsglldpsq amgptligla mgdaggykas dmwgpkedpa wqrndpllnv |
| 241 | gklianntrv wvycgngkps dlggnnlpak flegfvrtsn ikfqdaynag gghngvfdfp |
| 301 | dsgthsweyw gaqlnamkpd lqralgatpn tgpapqga |

which is encoded by the following nucleic acid sequence:

| | |
|---|---|
| 1 | cgacacatgc ccagacactg cggaaatgcc accttcaggc cgtcgcgtcg gtcccgaatt |
| 61 | ggccgtgaac gaccgccgga taagggtttc ggcggtgcgc ttgatgcggg tggacgcccg |
| 121 | aagttgtggt tgactacacg agcactgccg ggcccagcgc ctgcagtctg acctaattca |
| 181 | ggatgcgccc aaacatgcat ggatgcgttg agatgaggat gagggaagca agaatgcagc |
| 241 | ttgttgacag ggttcgtggc gccgtcacgg gtatgtcgcg tcgactcgtg gtcggggccg |
| 301 | tcggcgcggc cctagtgtcg ggtctggtcg gcgccgtcgg tggcacggcg accgcggggg |
| 361 | cattttcccg gccgggcttg ccggtggagt acctgcaggt gccgtcgccg tcgatgggcc |
| 421 | gtgacatcaa ggtccaattc caaagtggtg gtgccaactc gcccgccctg tacctgctcg |
| 481 | acggcctgcg cgcgcaggac gacttcagcg gctgggacat caacaccccg gcgttcgagt |
| 541 | ggtacgacca gtcgggcctg tcggtggtca tgccggtggg tggccagtca agcttctact |
| 601 | ccgactggta ccagcccgcc tgcggcaagg ccggttgcca gacttacaag tgggagacct |
| 661 | tcctgaccag cgagctgccg gggtggctgc aggccaacag gcacgtcaag cccaccggaa |
| 721 | gcgccgtcgt cggtctttcg atggctgctt cttcggcgct gacgctggcg atctatcacc |
| 781 | cccagcagtt cgtctacgcg ggagcgatgt cgggcctgtt ggacccctcc caggcgatgg |
| 841 | gtcccaccct gatcggcctg gcgatgggtg acgctggcgg ctacaaggcc tccgacatgt |
| 901 | ggggcccgaa ggaggacccg gcgtggcagc gcaacgaccc gctgttgaac gtcgggaagc |
| 961 | tgatcgccaa caacacccgc gtctgggtgt actgcggcaa cggcaagccg tcggatctgg |
| 1021 | gtggcaacaa cctgccggcc aagttcctcg agggcttcgt gcggaccagc aacatcaagt |
| 1081 | tccaagacgc ctacaacgcc ggtggcggcc acaacggcgt gttcgacttc ccggacagcg |
| 1141 | gtacgcacag ctgggagtac tggggcgcgc agctcaacgc tatgaagccc gacctgcaac |
| 1201 | gggcactggg tgccacgccc aacaccgggc ccgcgcccca gggcgcctag ctccgaacag |
| 1261 | acacaacatc tagcnncggt gacccttgtg gnncanatgt ttcctaaatc ccgtccctag |
| 1321 | ctcccgcngc nnccgtgtgg ttagctacct gacnncatgg gttt |

Ag85B (SwissProt accession no. P31952; aa 41-325). The amino acid and encoding nucleic acid sequences are provided below:

| | |
|---|---|
| 1 | mtdvsrkira wgrrlmigta aavvlpglvg laggaataga fsrpglpvey lqvpspsmgr |
| 61 | dikvqfqsgg nnspavylld glraqddyng wdintpafew yyqsglsivm pvggqssfys |
| 121 | dwyspacgka gcqtykwetf ltselpqwls anravkptgs aaiglsmags samilaayhp |
| 181 | qqfiyagsls alldpsqgmg psliglamgd aggykaadmw gpssdpawer ndptqqipkl |
| 241 | vanntrlwvy cgngtpnelg ganipaefle nfvrssnlkf qdaynaaggh navfnfppng |
| 301 | thsweywgaq lnamkgdlqs slgag |
| 1 | atgacagacg tgagccgaaa gattcgagct tggggacgcc gattgatgat cggcacggca |
| 61 | gcggctgtag tccttccggg cctggtgggg cttgccggcg gagcggcaac cgcgggcgcg |
| 121 | ttctcccggc cggggctgcc ggtcgagtac ctgcaggtgc cgtcgccgtc gatgggccgc |
| 181 | gacatcaagg ttcagttcca gagcggtggg aacaactcac ctgcggttta tctgctcgac |
| 241 | ggcctgcgcg cccaagacga ctacaacggc tgggatatca acaccccggc gttcgagtgg |
| 301 | tactaccagt cgggactgtc gatagtcatg ccggtcggcg ggcagtccag cttctacagc |
| 361 | gactggtaca gcccggcctg cggtaaggct ggctgccaga cttacaagtg ggaaaccttc |
| 421 | ctgaccagcg agctgccgca atggttgtcc gccaacaggg ccgtgaagcc caccggcagc |
| 481 | gctgcaatcg gcttgtcgat ggccggctcg tcggcaatga tcttggccgc ctaccacccc |
| 541 | cagcagttca tctacgccgg ctcgctgtcg gccctgctgg acccctctca ggggatgggg |
| 601 | cctagcctga tcggcctcgc gatgggtgac gccggcggtt acaaggccgc agacatgtgg |
| 661 | ggtccctcga gtgacccggc atgggagcgc aacgacccta cgcagcagat ccccaagctg |
| 721 | gtcgcaaaca acacccggct atgggtttat tgcgggaacg gcaccccgaa cgagttgggc |
| 781 | ggtgccaaca tacccgccga gttcttggag aacttcgttc gtagcagcaa cctgaagttc |
| 841 | caggatgcgt acaacgccgc gggcgggcac aacgccgtgt tcaacttccc gcccaacggc |
| 901 | acgcacagct gggagtactg gggcgctcag ctcaacgcca tgaagggtga cctgcagagt |
| 961 | tcgttaggcg ccggctga |

and TB 10.4 (SwissProt accession no. 053693; full sequence). The amino acid and nucleic acid sequences are provided below:

| 1 | msqimynypa mlghagdmag yagtlqslga eiaveqaalq sawqgdtgit yqawqaqwnq |
|---|---|
| 61 | amedlvrayh amsstheant mammardtae aakwgg |
| NC_000962.2 | |
| 1 | atgtcgcaaa tcatgtacaa ctaccccgcg atgttgggtc acgccgggga tatggccgga |
| 61 | tatgccggca cgctgcagag cttgggtgcc gagatcgccg tggagcaggc cgcgttgcag |
| 121 | agtgcgtggc agggcgatac cgggatcacg tatcaggcgt ggcaggcaca gtggaaccag |
| 181 | gccatggaag atttggtgcg ggcctatcat gcgatgtcca gcacccatga agccaacacc |
| 241 | atggcgatga tggcccgcga cacggccgaa gccgccaaat ggggcggcta g |

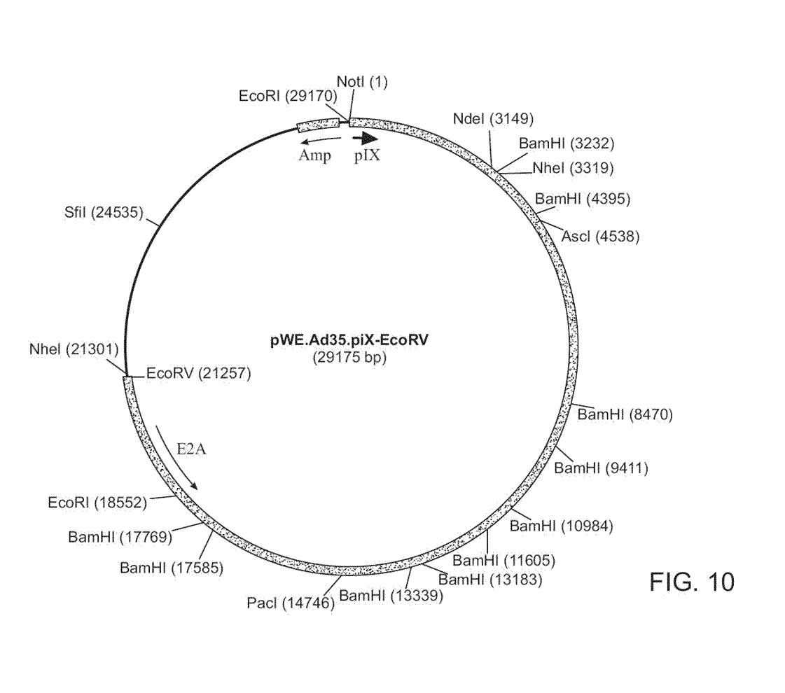

The three coding sequences are linked directly, thus without signal peptides or intervening sequences, in the order Ag85A-Ag85B-TB 10.4. Sequences were cloned unidirectionally into the pAdap35.Bsu plasmids and vectors carrying Ad5-derived Orf6 were generated. An exemplary map for a E1/E3-deleted Ad35 based vector, which is described in US 2008/0199917 is provided at FIG. 10.

Adenovirus from various origins, subtypes, or mixture of subtypes can be used as the source of the viral genome for the adenoviral vector. Non-human adenovirus (e.g., simian, avian, canine, ovine, or bovine adenoviruses) can be used to generate the adenoviral vector. In another embodiment, a human adenovirus is used as the source of the viral genome for the adenoviral vector. Adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561 and International Patent Applications WO 97/12986 and WO 98/53087.

Preferably, adenoviral vectors useful in the invention are replication-deficient in host cells. By “replication-deficient” is meant that the adenoviral vector will not replicate in a host cell. A replication deficient adenoviral vector requires complementation of one or more regions of the adenoviral genome that are required for replication, as a result of, for example a deficiency in at least one replication-essential gene function (i.e., such that the adenoviral vector does not replicate in typical host cells, especially those in a human patient that could be infected by the adenoviral vector). Suitable replication-deficient adenoviral vectors, including multiply replication-deficient adenoviral vectors, are disclosed in U.S. Pat. Nos. 5,837,511; 5,851,806; 5,994,106; 6,127,175; and 6,482,616; U.S. Patent Application Publications 2001/0043922 A1, 2002/0004040 A1, 2002/0031831 A1, 2002/0110545 A1, and 2004/0161848 A1, and International Patent Application Publications WO 94/28152, WO 95/02697, WO 95/16772, WO 95/34671, WO 96/22378, WO 97/12986, WO 97/21826, and WO 03/022311.

Although the Examples specifically describe the use of recombinant adenoviral (rAd) vectors (e.g., rAd5, rAd35) encoding SIV Env, Pol, and Gag polypeptides, influenza hemagglutinin polypeptides, and TB antigens 85A/B and 10.4, one of skill in the art will appreciate that the invention is not so limited. Adeno-associated viruses, retroviruses, lentiviruses, and liposomal vectors have all shown promise in preclinical studies in the lung, and some have been tested in clinical trials. Moreover, most of these vectors can be delivered to the human airway epithelium relatively safely. In other embodiments, genetic vaccines of the invention employ human parainfluenza virus type 1, human coronavirus 229E, paramxyovirus (PV), respiratory syncytial virus (RSV), and human parainfluenza virus (PIV)-based vectors. The invention is readily adaptable, and may be used with any viral vector suitable for expression in a mammalian cell, as well as with any antigen suitable for inducing a desired immune response.

Immunogenic compositions of the invention may affect one or more aspects of the immune response in a subject. Immunogenic compositions of the invention can affect a cell mediated immune response and/or a humoral immune response. Aspects of the immune response include, but are not limited to, the inflammatory response, the complement cascade, leukocyte and lymphocyte differentiation, proliferation, and/or effectors function, monocytes and/or basophil counts, and the cellular communication among cells of the immune system. In certain embodiments of the invention, an immunogenic composition modulates one aspect of the immune response. In other embodiments, an immunogenic composition modulates more than one aspect of the immune response, e.g. a cell mediated immune response and a humoral immune response.

In one embodiment, a recombinant adenoviral vector encoding a pathogen polypeptide is prepared. Methods for inserting a desired polypeptide into an adenoviral vector are known in the art. See, for example, Santra et al., “Replication-defective adenovirus serotype 5 vectors elicit durable cellular and humoral immune responses in nonhuman primates,” J Virol. 2005 May; 79(10):6516-22; and Ohno et al., “Gene therapy for vascular smooth muscle cell proliferation after arterial injury,” Science, Vol 265, Issue 5173, 781-784. In general, adenoviral vectors are prepared from two components: a viral DNA vector and a packaging cell line. The adenoviral DNA vector is a plasmid DNA that contains a portion of the viral genome. It has had the E1A region deleted, and desired genes are cloned into a multicloning site that has been inserted in place of the E1A region of the genome. Because the large size of the adenoviral genome limits the use of a single plasmid-based system in vector production, the adenoviral vector is typically produced using either in vitro ligation or homologous recombination. Using in vitro ligation, wild-type adenoviral DNA is isolated and cleaved with a desired restriction endonuclease (e.g., ClaI). The digested viral DNA is then ligated onto the adenoviral DNA vector containing the gene of interest which has previously been digested with the restriction endonuclease. The ligated DNA is then transfected into a packaging cell line. Both the adenoviral DNA vector and the viral DNA component are co-transfected into a packaging cell line. These DNA species then undergo homologous recombination resulting in vector production (Wolff, J A. Gene Therapeutics, 1994. p. 347).

Preferably, a transducing viral vector (e.g., retroviral, adenoviral, and adeno-associated viral) is used that is capable of infecting cells found in the lung (e.g., epithelial cells, macrophages, dendritic cells). Vectors capable of infecting such cells are known in the art (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). In one embodiment, a polynucleotide encoding a pathogen polypeptide or a fragment thereof, is cloned into a viral vector, and expression is driven from an endogenous promoter, from a retroviral long terminal repeat, or from a promoter specific for a target cell type of interest (e.g., mucosa, such as lung epithelial cells, macrophages, dendritic cells). Other viral vectors that can be used in the methods of the invention include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are well developed and have been used in clinical settings (Rosenberg et al., N. Engl. J. Med 323:370, 1990; Anderson et al., U.S. Pat. No. 5,399,346).

As described herein, the invention provides formulations containing gene constructs which, when expressed, produce a protein. This protein may be one which the patient is in need of, or it may stimulate an immune response so that the patient is thereby vaccinated. The protein may also be one that induces immunological tolerance, or which produces an enzyme which digests unwanted mucus comprised, in part, of DNA. The protein may also be one that provides a detectable signal, and is thus useful in diagnostic methods.

In some embodiments, polynucleotides are contained within, or associated with, non-viral delivery vehicles. In some of these embodiments, the polynucleotide is delivered naked or formulated and condensed with a carrier. In other embodiments, the polynucleotide is associated with (complexed with) an artificial viral envelope. It is not desirable, in some cases, to deliver the genetic material to the outermost areas of the lungs where gas transfer takes place. By adjusting various parameters, particularly particle size, but also optionally particle density, inspiratory flow rate, the inspired volume when the aerosol “bolus” is delivered, and the total volume inhaled, specific locations within the respiratory tract, may be targeted. Thus, by methods described herein, it is possible to deliver the genetic material to the desired region(s) of the respiratory tract. When the genetic material is brought into contact with the mucous membranes of the central regions of the lungs or the peripheral gas exchange areas of the lungs or pulmonary macrophages and other cells of the respiratory tract, the material migrates into cells where it is expressed and thereafter the product of the expression delivered to the patient. Alternatively, the polynucleotides, with or without the vehicles, can migrate into the lymph or blood circulation to target other sites in the body.

Any of the immunogenic compositions as described herein are capable of generating an immune response against an infectious agent.

The methods of the invention can also be used to deliver drugs that cannot be administered orally due to lack of solubility and bio-availability or poor absorption or degradation of drugs in the gastrointestinal tract. Furthermore, the methods of the invention can be used for the systemic administration of drugs which usually require parenteral administration by the intravenous (iv.), intramuscular (im), subcutaneous (sc), and intrathecal route.

Thus, the invention, and in particular use of an aerosol nebulizer, allows both topical and systemic aerosol drug delivery via either the nasal or the pulmonary route for a wide variety of drugs that can be formulated or prepared in-situ or immediately before use as solution, suspension or emulsion or any other pharmaceutical application system, such as liposomes or nanosomes. The nebulizer can be modified with respect to the pore size and dimension of the mixing chamber to direct aerosol delivery either into the nose or lungs. Therefore, various droplet and particle sizes can be generated which can deliver an aerosolized immunogenic particles with a size distribution between 0.01 um and 15 um.

Viral vectors of the invention comprise polynucleotides encoding pathogenic polypeptides useful for generating a prophylactic or therapeutic immune response. Such pathogenic polypeptides are known in the art and exemplary pathogenic proteins are described herein.

### Recombinant Pathogen Polypeptides

The invention provides aerosol formulations of genetic vaccines that contain expression vectors that include polynucleotides encoding a pathogen polypeptide or a polypeptide having at least about 85% amino acid sequence identity to a reference polypeptide. Preferably, such vaccines are capable of inducing a therapeutic or prophylactic immune response against the pathogen. The invention describes the use of particular pathogen polypeptides useful in the treatment or prevention of tuberculosis, HIV/AIDS, SIV, and influenza. One of skill in the art will readily appreciate that the particular polypeptides described are merely exemplary and are in no way intended to limit the invention.

In one embodiment, the invention provides an aerosol formulation of an expression vector (e.g., adenoviral vector) encoding a tuberculosis antigen (e.g., 85A/B or 10.4). For example, the invention provides a genetic vaccine comprising an expression vector encoding tuberculosis (TB) antigen 85A (Huygen et al., Infect. Immun. 57 (10), 3123-3130 (1989). The sequence of an exemplary TB 85A polypeptide (NCBI Accession No. P0A4V2) is provided below:

| | |
|---|---|
| 1 | mqlvdrvrga vtgmsrrlvv gavgaalvsg lvgavggtat agafsrpglp veylqvpsps |
| 61 | mgrdikvqfq sgganspaly lldglraqdd fsgwdintpa fewydqsgls vvmpvggqss |
| 121 | fysdwyqpac gkagcqtykw etfltselpg wlqanrhvkp tgsavvglsm aassaltlai |
| 181 | yhpqqfvyag amsglldpsq amgptligla mgdaggykas dmwgpkedpa wqrndpllnv |
| 241 | gklianntrv wvycgngkps dlggnnlpak flegfvrtsn ikfqdaynag gghngvfdfp |
| 301 | dsgthsweyw gaqlnamkpd lqralgatpn tgpapqga |

In another embodiment, the invention provides an aerosol formulation of a vaccine that contains an expression vector encoding tuberculosis (TB) 85B. The sequence of an exemplary TB antigen 85B (NCBI Accession No. NP 216402) is provided below:

| | |
|---|---|
| 1 | mtdvsrkira wgrrlmigta aavvlpglvg laggaataga fsrpglpvey lqvpspsmgr |
| 61 | dikvqfqsgg nnspavylld glraqddyng wdintpafew yyqsglsivm pvggqssfys |
| 121 | dwyspacgka gcqtykwetf ltselpqwls anravkptgs aaiglsmags samilaayhp |
| 181 | qqfiyagsls alldpsqgmg psliglamgd aggykaadmw gpssdpawer ndptqqipkl |
| 241 | vanntrlwvy cgngtpnelg ganipaefle nfvrssnlkf qdaynaaggh navfnfppng |
| 301 | thsweywgaq lnamkgdlqs slgag |

In another embodiment, the invention provides an aerosol formulation of a vaccine that contains an expression vector encoding TB 10.4 (NCBI Accession No. P0A568). TB 10.4 is described by Skjot et al., Infect. Immun. 68 (1), 214-220 (2000). The sequence of an exemplary TB 10.4 is provided below:

| | |
|---|---|
| 1 | msqlmynypa mlghagdmag yagtlqslga eiaveqaalq sawqgdtgit yqawqaqwnq |
| 61 | amedlvrayh amsstheant mammardtae aakwgg. |

Adenoviral vectors encoding TB polypeptides are known in the art and described, for example, by Radosevic, K. et al., Infect Immun 75 (8), 4105 (2007), which is incorporated by reference in its entirety.

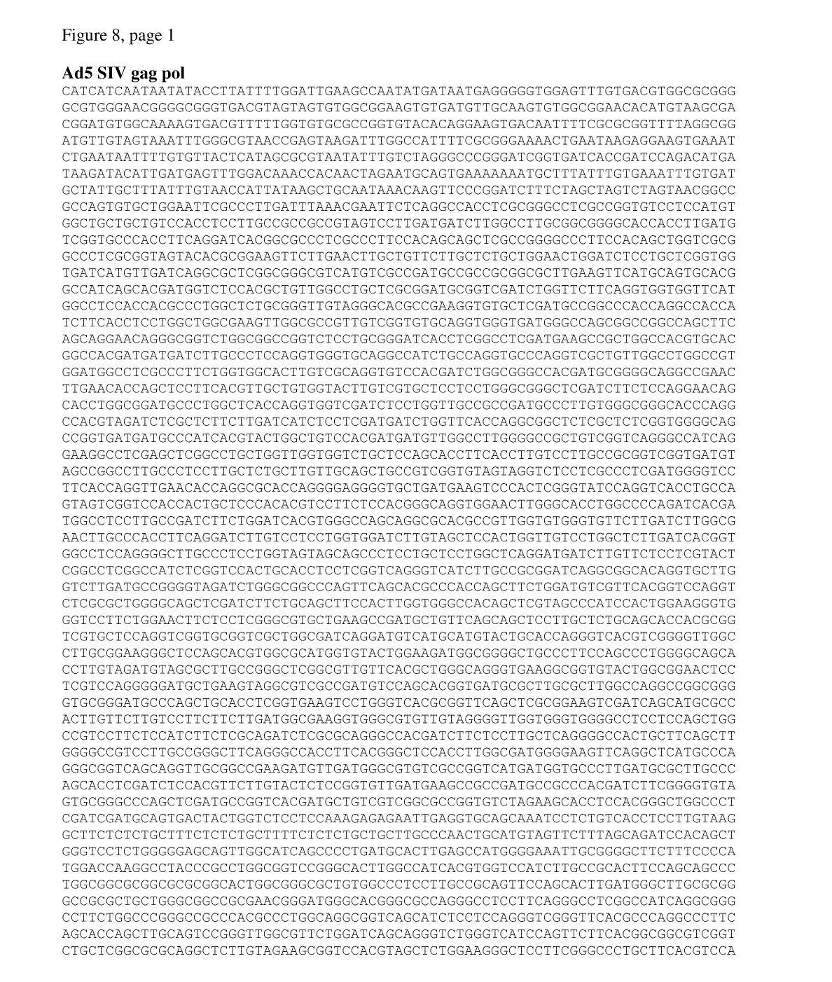

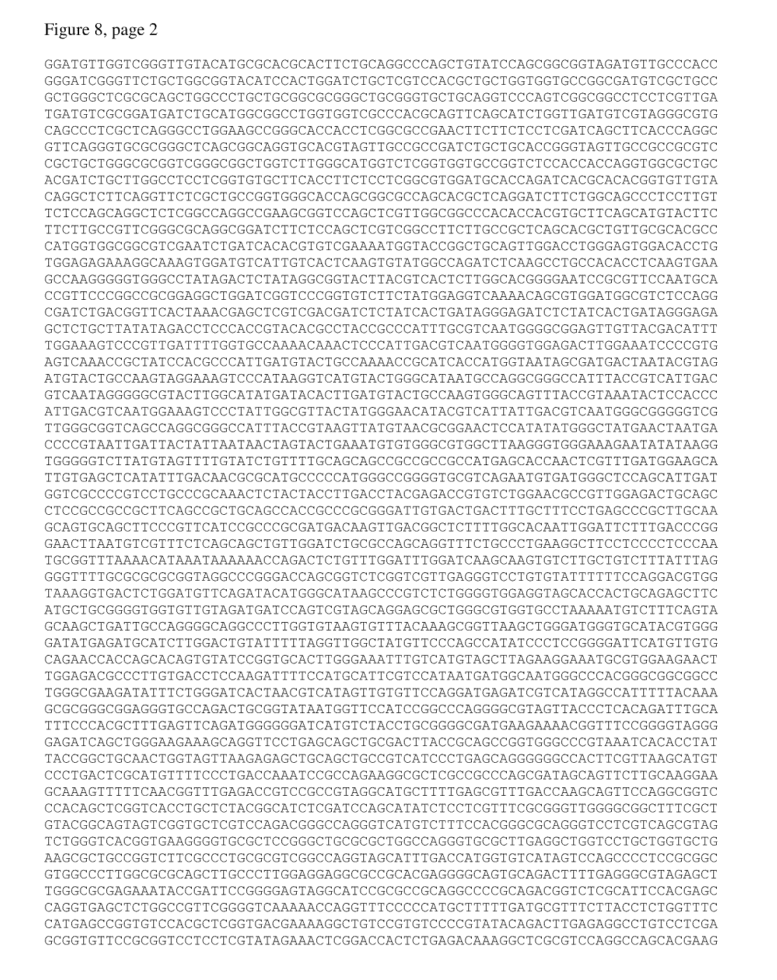

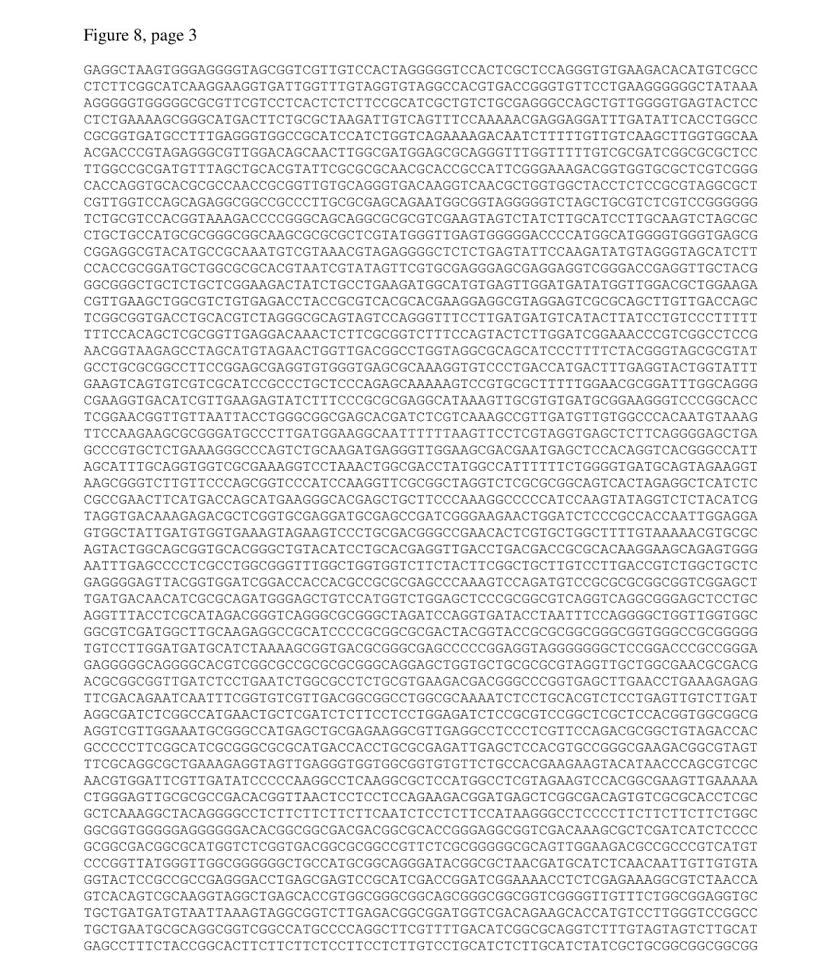

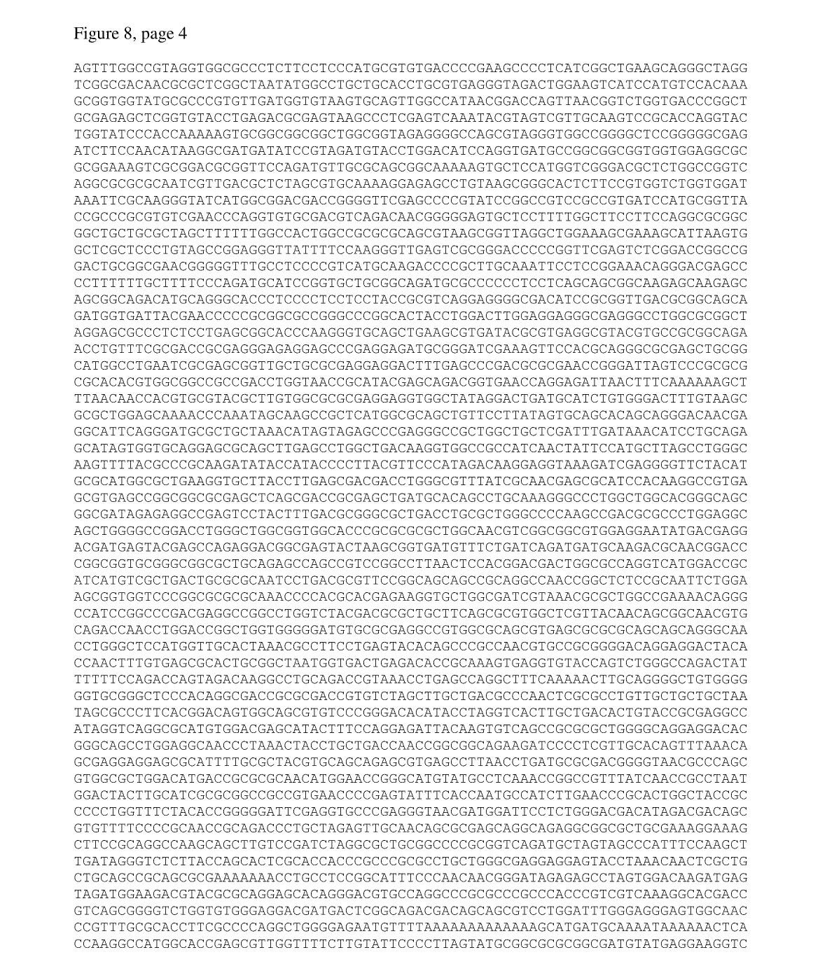

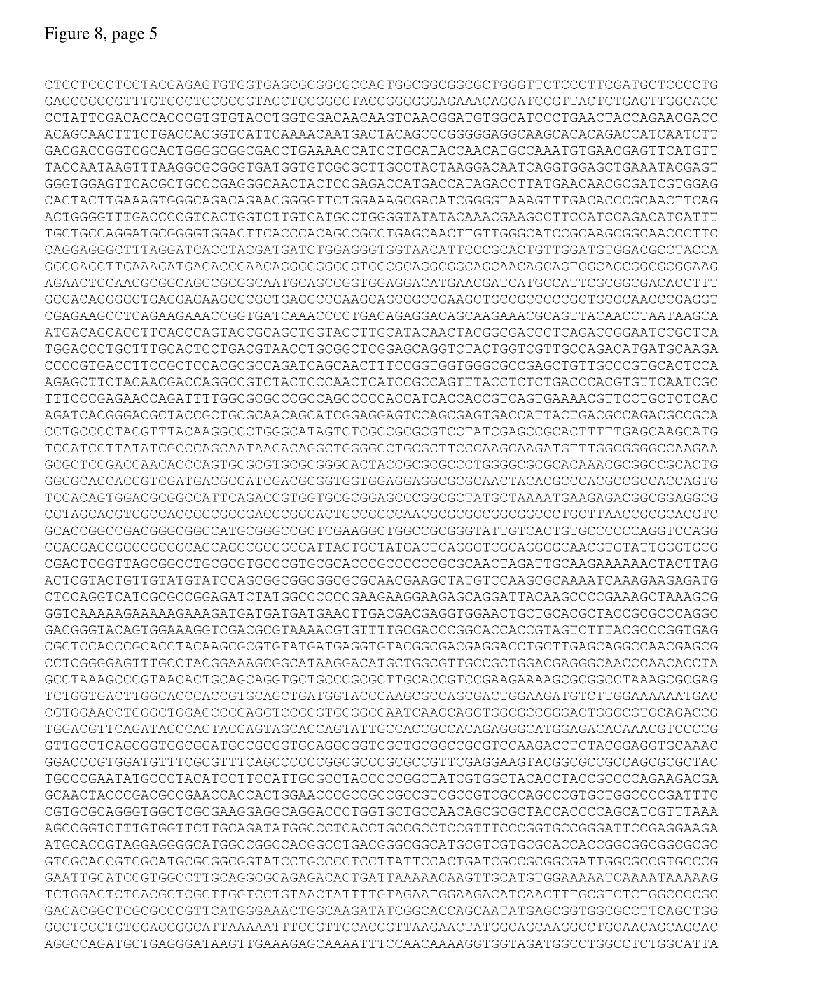

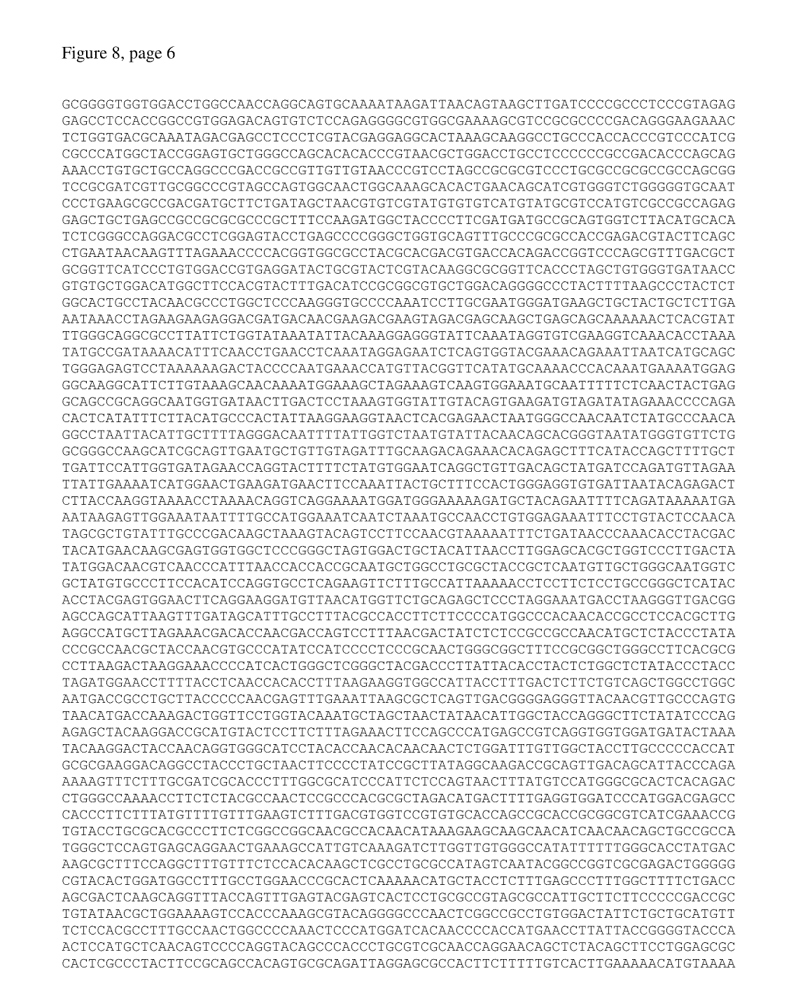

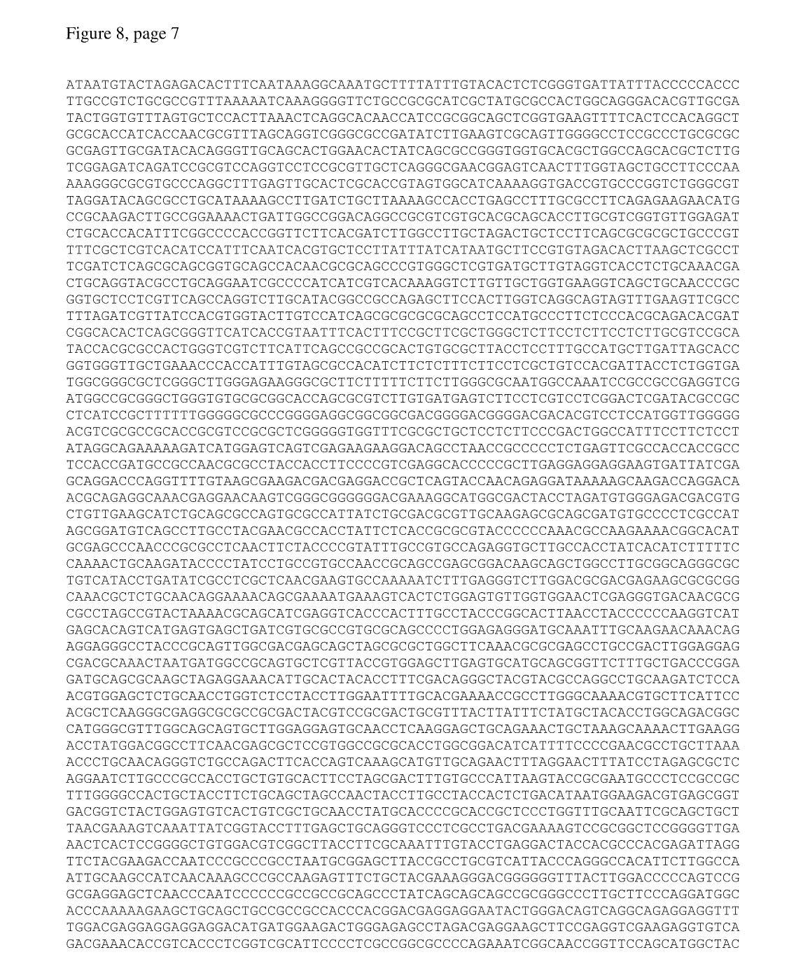

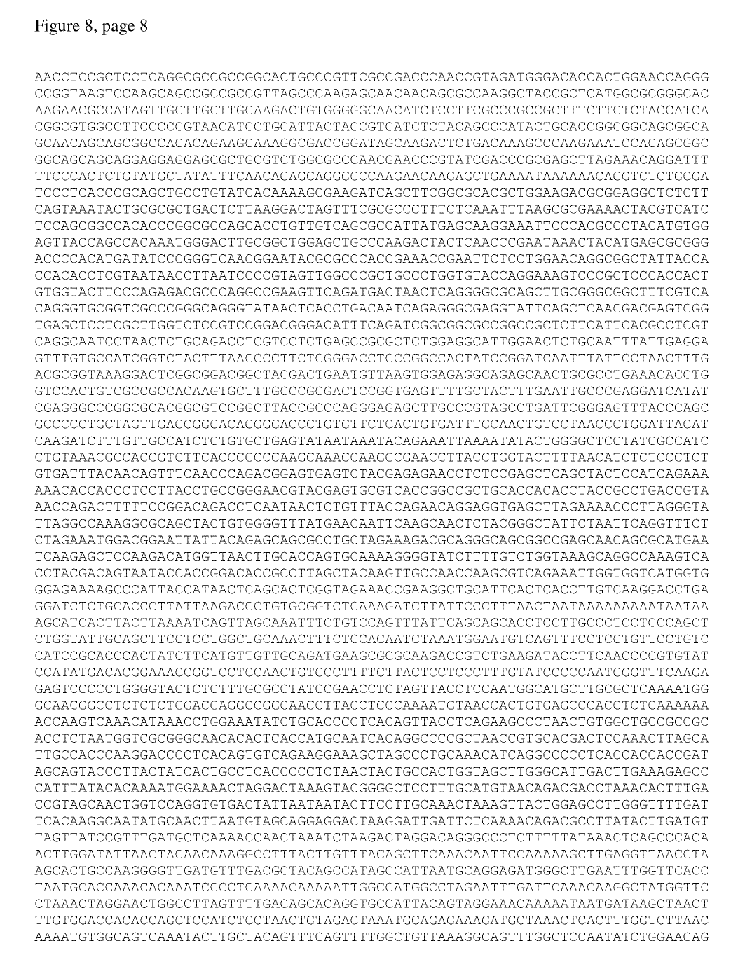

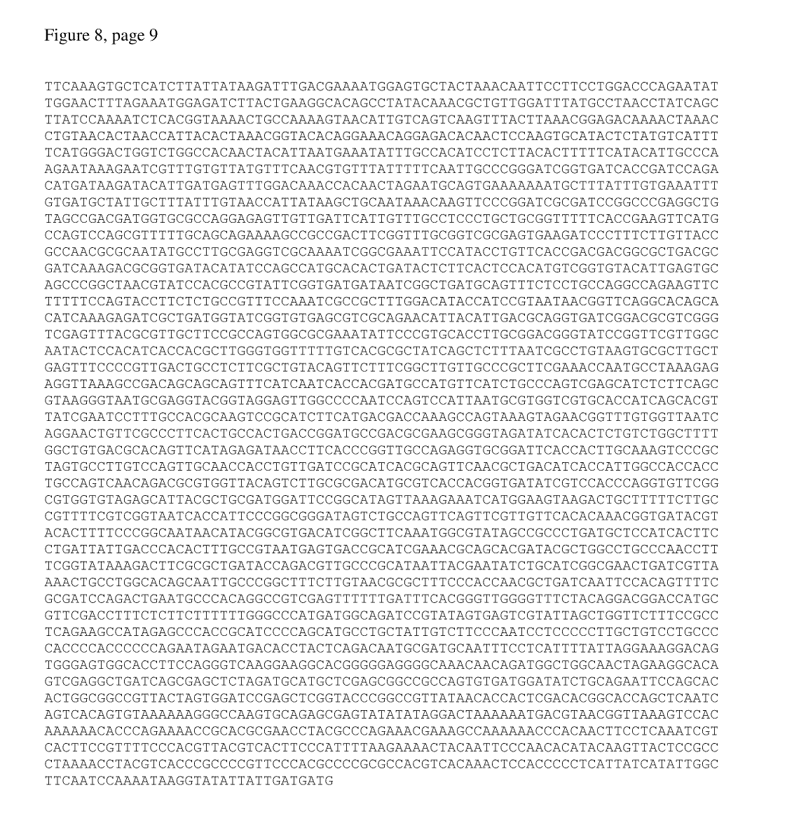

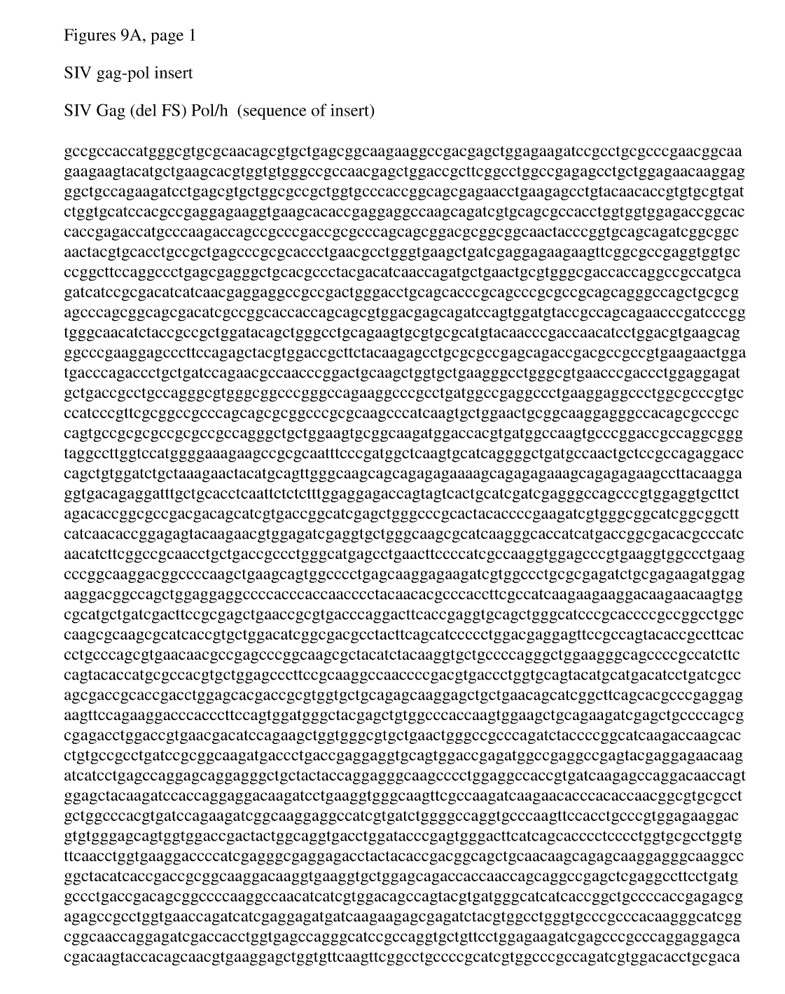

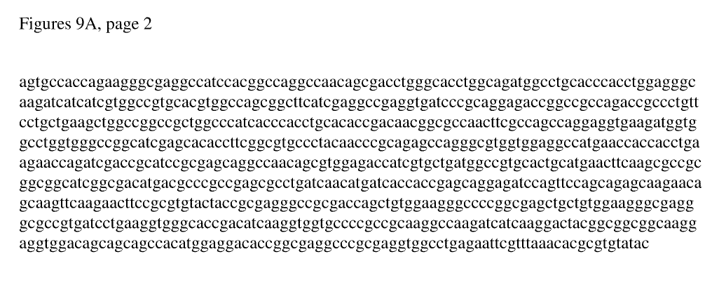

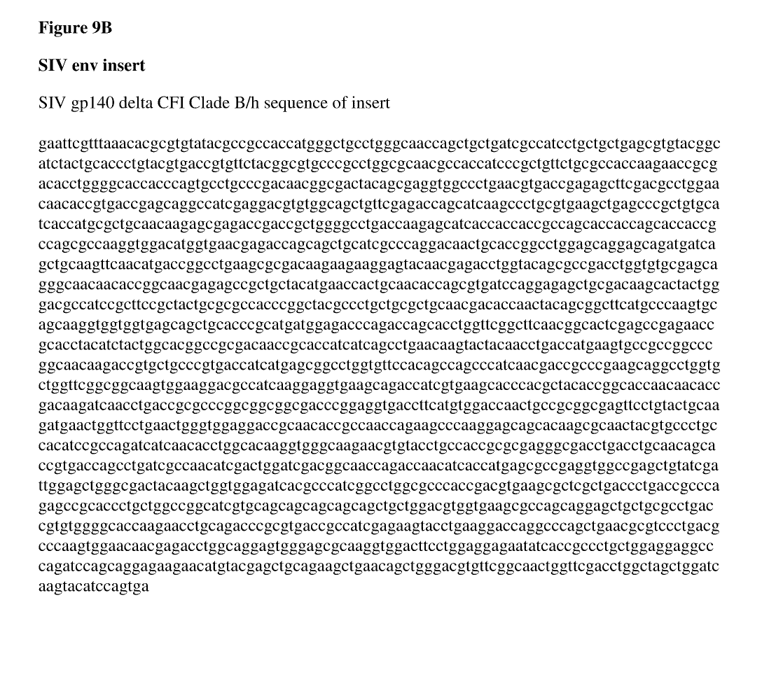

Exemplary adenoviral vectors encoding SIV polypeptides are described, for example, by Sun et al., “Virus-Specific Cellular Immune Correlates of Survival in Vaccinated Monkeys after Simian Immunodeficiency Virus Challenge,” Journal of Virology, 80:10950-10956, 2006, which is incorporated by reference in its entirety. Exemplary sequences of SIV Env and SIV Gag-Pol polypeptides are provided at FIGS. 8 (Ad5 SIV env), 9A and 9B.

The invention further provides an aerosol formulation of a vaccine that contains an expression vector encoding an influenza polypeptide, such as hemagglutinin (HA) (e.g., HA from Influenza A, such as NCBI Accession No. ABM90500, an HA from Influenza B, such as NP 056660). For example, the invention provides an adenoviral vector comprising an exemplary influenza hemagglutinin polypeptide. The sequence of an exemplary Influenza hemagglutinin polypeptide from Influenza A virus (A/Indonesia/CDC1032T/2007(H5N1) (NCBI Accession No. ABM90500) is provided below:

| | |
|---|---|
| 1 | dqicigyhan nsteqvdtim eknvtvthaq dilekthngk lcdldgvkpl ilrdcsvagw |
| 61 | llgnpmcdef invpewsyiv ekanptndlc ypgsfndyee lkhllsrinh fekiqiipks |
| 121 | swsdheassg vssacpylgs psffrnvvwl ikknstypti kksynntnqe dllvlwgihh |
| 181 | pnneeeqtrl yqnpttyisi gtstlnqrlv pkiatrskvn gqsgrmeffw tilkpndain |
| 241 | fesngnfiap eyaykivkkg dsaimksele ysncntkcqt pmgainssmp fhnihpltig |
| 301 | ecpkyvkssr lvlatglrns pqresrrkkr glfgaiagfi eggwqgmvdg wygyhhsneq |
| 361 | gsgyaadkes tqkaidgvtn kvnsiidkmn tqfeavgref nnlerrienl nkkmedgfld |
| 421 | vwtynaellv lmenertldf hdsnvknlyd kvrlqlrdna kelgngcfef yhkcdnecme |
| 481 | sirngtynyp qyseearlkr eeisgvkles igtyqilsiy stvasslala imiaglslwm |
| 541 | csngslqcri ci |

The invention further provides an aerosol formulation of a vaccine that contains an expression vector encoding an HIV polypeptide. Examples of suitable HIV polypeptides include all or part of an HIV Gag, Env, Pol, Tat, Reverse Transcriptase (RT), Vif, Vpr, Vpu, Vpo, Integrase, or Nef proteins. Preferably, each of the two or more HIV polypeptides comprises all or part of an HIV Gag, Env, and/or Pol protein. Suitable Env proteins are known in the art and include, for example, gp160, gp120, gp41, gp145, and gp140. In addition, an HIV antigen can be a modified Env protein that exhibits enhanced immunogenicity in vivo. For example, the antigen can be an Env protein comprising mutations in the cleavage site, fusion peptide, or interhelical coiled-coil domains of the Env protein (see, e.g., Cao et al., J Virol., 71, 9808-9812 (1997), and Yang et al., J. Virol., 78, 4029-4036 (2004)).

Any clade of HIV is appropriate for antigen selection, including HIV clades A, B, C, D, B, MN, and the like. Thus, it will be appreciated that the following HIV antigens can be used in the compositions and methods of the invention: HIV clade A gp 140, Gag, Env, and/or Pol; HIV clade B gp140, Gag, Env, and/or Pol proteins; HIV clade C gp140, Gag, Env, and/or Pol proteins; and HIV clade MN gp140, Gag, Env, and/or Pol proteins. While it is preferred that the antigen is a Gag, Env, and/or Pol protein, any HIV polypeptide or portion thereof capable of inducing an immune response in a mammal can be used in connection with compositions and methods of the invention. HIV Gag, Env, and Pol proteins from the different HIV clades (e.g., HIV clades A, B, C, MN, etc.), as well as nucleic acid sequences encoding such proteins and methods for the manipulation and insertion of such nucleic acid sequences into vectors, are known (see, e.g., HIV Sequence Compendium, Division of AIDS, National Institute of Allergy and Infectious Diseases (2003), HIV Sequence Database (http://hiv-web.1an1.gov/content/hiv-db/mainpage.html)

The amino acid sequence of an exemplary HIV GAG polypeptide (NCBI Accession No. AAB50258) is provided below:

| GAG polyprotein [Human immunodeficiency virus 1]. | |
|---|---|
| 1 | mgarasvlsg geldrwekir lrpggkkkyk lkhivwasre lerfavnpgl letsegcrqi |
| 61 | lgqlqpslqt gseelrslyn tvatlycvhq rieikdtkea ldkieeeqnk skkkaqqaaa |
| 121 | dtghsnqvsq nypivqniqg qmvhqaispr tlnawvkvve ekafspevip mfsalsegat |
| 181 | pqdlntmlnt vgghqaamqm lketineeaa ewdrvhpvha gpiapgqmre prgsdiagtt |
| 241 | stlqeqigwm tnnppipvge iykrwiilgl nkivrmyspt sildirqgpk epfrdyvdrf |
| 301 | yktlraeqas qevknwmtet llvqnanpdc ktilkalgpa atleemmtac qgvggpghka |
| 361 | rvlaeamsqv tnsatimmqr gnfrnqrkiv kcfncgkegh tarncraprk kgcwkcgkeg |
| 421 | hqmkdcterq anflgkiwps ykgrpgnflq srpeptappe esfrsgvett tppqkqepid |
| 481 | kelypltslr slfgndpssq |

The amino acid sequence of an exemplary HIV envelope glycoprotein [Human immunodeficiency virus type 1] (NCBI Accession No. AAB05174) is provided below:

| | |
|---|---|
| 1 | vrgiqtswqn lwrwgtmilg mlviysaaen lwvavyygvp vwkdaettlf casdakaydt |
| 61 | evhnvwetha cvptdpnpqe ihlenvtedf nmwrnnmveq mhtdiislwd qslkpcvklt |
| 121 | plcvtldcna tasnvtnemr ncsfnittel kdkkqqvysl fykldvvqin eknetdkyrl |
| 181 | incntsaitq acpkvsfepi pihycapagf ailkckdtef ngtgpcknvs tvqcthgirp |
| 241 | vistqlllng slaeegiqir senitnnakt iivqldkavk inctrpnnnt rkgvrigpgq |
| 301 | afyatggiig dirqahchvs rakwndtlrg vakklrehfk nktiifekss ggdieitths |
| 361 | ficggeffyc ntsglfnstw esnstesnnt tsndtitltc rikqiinmwq kvgqamyppp |
| 421 | iqgvircesn itgllltrdg gnnstneifr pgggnmrdnw rselykykvv kieplgvaps |
| 481 | rakrrvvere kravgigavf lgflgaagst mgaasitlta qarqllsgiv qqqsnllrai |
| 541 | eaqqhmlklt vwgikqlqar vlaverylkd qqllgiwgcs gklicttnvp wnsswsnksm |
| 601 | neiwdnmtwl qwdkeisnyt qiiynliees qnqqekneqd llaldkwasl wnwfdisrwl |
| 661 | wyikifimiv ggliglrivf avlsvinrvr qgysplsfqi rtpnpkepdr lgridgegge |
| 721 | qdrdrsirlv sgflalawdd lrslclfsyh rlrdfisiaa rtvellghss lkglrlgweg |
| 781 | lkylwnllly wgrelktsav nlvdtiaiav agrtdrviev gqrifrailn iprrirqgle |
| 841 | rgll |

The amino acid sequence of an exemplary HIV Pol polypeptide (HIV-1 vector pNL4-3, NCBI Accession No. AAK08484) is provided below:

| | |
|---|---|
| 1 | ffredlafpq gkarefsseq transptrre lqvwgrdnns lseagadrqg tvsfsfpqit |
| 61 | lwqrplvtik iggqlkeall dtgaddtvle emnlpgrwkp kmiggiggfi kvrqydqili |
| 121 | eicghkaigt vlvgptpvni igrnlltqig ctlnfpispi etvpvklkpg mdgpkvkqwp |
| 181 | lteekikalv eictemekeg kiskigpenp yntpvfaikk kdstkwrklv dfrelnkrtq |
| 241 | dfwevqlgip hpaglkqkks vtvldvgday fsvpldkdfr kytaftipsi nnetpgiryq |
| 301 | ynvlpqgwkg spaifqcsmt kilepfrkqn pdiviyqymd dlyvgsdlei gqhrtkieel |
| 361 | rqhllrwgft tpdkkhqkep pflwmgyelh pdkwtvqpiv lpekdswtvn diqklvgkln |
| 421 | wasqiyagik vrqlckllrg tkaltevvpl teeaelelae nreilkepvh gvyydpskdl |
| 481 | iaeiqkqgqg qwtyqiyqep fknlktgkya rmkgahtndv kqlteavqki atesiviwgk |
| 541 | tpkfklpiqk etweawwtey wqatwipewe fvntpplvkl wyqlekepii gaetfyvdga |
| 601 | anretklgka gyvtdrgrqk vvpltdttnq ktelqaihla lqdsglevni vtdsqyalgi |
| 661 | iqaqpdkses elvsqiieql ikkekvylaw vpahkgiggn eqvdglvsag irkvlfldgi |
| 721 | dkaqeeheky hsnwramasd fnlppvvake ivascdkcql kgeamhgqvd cspgiwqldc |
| 781 | thlegkvilv avhvasgyie aevipaetgq etayfllkla grwpvktvht dngsnftstt |
| 841 | vkaacwwagi kqefgipynp qsqgviesmn kelkkiigqv rdqaehlkta vqmavfihnf |
| 901 | krkggiggys agerivdiia tdiqtkelqk qitkiqnfry yyrdsrdpvw kgpakllwkg |
| 961 | egavviqdns dikvvprrka kiirdygkqm agddcvasrq ded |

The sequence and map of an exemplary rAd5 vector encoding HIV envA is described, for example, at WO/2006/020071, entitled “Vaccines Against AIDS Comprising CMV/R-Nucleic Acid Constructs.”

Many modifications and variations of the present exemplary DNA sequences and plasmids are possible. For example, the degeneracy of the genetic code allows for the substitution of nucleotides throughout polypeptide coding regions, as well as in the translational stop signal, without alteration of the encoded polypeptide coding sequence. Such substitutable sequences can be deduced from the known amino acid or DNA sequence of a given gene and can be constructed by conventional synthetic or site-specific mutagenesis procedures. Synthetic DNA methods can be carried out in substantial accordance with the procedures of Itakura et al., Science, 198, 1056 (1977) and Crea et al., PNAS USA, 75, 5765 (1978). Site-specific mutagenesis procedures are described in Maniatis et al., Molecular Cloning: A Laboratory Manual, Cold Spring Harbor, N.Y. (2d ed. 1989). The choice of gene or DNA sequence should be one that will achieve a therapeutic effect, for example, in the context of gene therapy, vaccination, and the like. One of skill in the art will appreciate that virtually any pathogen polypeptide known in the art may be inserted in an expression vector for use as an aerosolized genetic vaccine. Such vaccines are useful for the treatment or prevention of an infection with virtually any pathogen known in the art.

### Pathogens

Compositions (e.g., aerosolized genetic vaccines) and related methods of the invention are useful for generating a prophylactic or therapeutic immune response against a pathogen. Pathogens include, but are not limited to, bacteria, viruses, yeast and fungi. Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, Corynebacterium diphtheriae, Corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

Both gram negative and gram positive bacteria may act as pathogens in vertebrate animals. Gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. Tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of viruses that have been found in humans include, but are not limited to, Retroviridae (e.g. human immunodeficiency viruses, such as HIV-1 (also referred to as HDTV-III, LAVE or HTLV-IIIILAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. The agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of pathogenic fungi include, without limitation, Alternaria, Aspergillus, Basidiobolus, Bipolaris, Blastomyces dermatitidis, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, Candida lusitaniae, Coccidioides, Coccidioides immitis, Cladophialophora, Chlamydia trachomatis, Candida albicans, Cryptococcus, Cryptococcus neoformans, Cunninghamella, Curvularia, Exophiala, Fonsecaea, Histoplasma, Histoplasma capsulatum, Madurella, Malassezia, Plastomyces, Rhodotorula, Scedosporium, Scopulariopsis, Sporobolomyces, Tinea, and Trichosporon.

In certain preferred embodiments, the pathogen infects the respiratory tract. A respiratory infectious agent may be a virus (e.g., a respiratory virus), a bacterium or a fungus. In particular embodiments, the invention provides for the treatment or prevention of an infection with a respiratory virus (e.g., influenza viruses, parainfluenza viruses, respiratory syncytial viruses (RSV), adenoviruses, rhinoviruses, and severe acute respiratory syndrome (SARS) viruses). In particular embodiments, methods of the invention are useful for the treatment or prevention of a respiratory infection associated with influenza virus type A, influenza virus type B, influenza virus type C, parainfluenza virus type 1, parainfluenza virus type 2, parainfluenza virus type 3, respiratory syncytial virus (RSV), a respiratory coronavirus, or a respiratory adenovirus.

In other embodiments, the invention provides for the treatment or prevention of an infection associated with Streptococcus pyogenes, Haemophilus influenzae, Corynebacterium diphtheriae, Bordetella pertussis, Moraxella catarrhalis, Rhinovirus, Coronavirus, Adenovirus, Respiratory Syncytial Virus (RSV), Parainfluenza virus, Mumps virus, Streptococcus pneumoniae (pneumococcus), Staphylococcus aureus, Legionella pneumophila, Klebsiella pneumoniae, Pseudomonas aeruginosa, Burkholderia cepacia, Mycoplasma pneumoniae, Mycobacterium tuberculosis, Chlamydia Pneumonia, Mycobacterium avium intracellulare complex (MAC), Candida albicans, Coccidioides immitis, Histoplama capsulatum, Blastomyces dermatitidis, Cryptococcus neoformans, and Aspergillus fumigatus. The infectious agent may be Pneuocystis carinii. The infectious agent may be a rickettsia that causes Q fever or typhus. Preferably, the respiratory infectious agent is Mycobacterium tuberculosis, influenza virus, parainfluenza virus, respiratory syncytial virus (RSV), respiratory coronavirus, respiratory adenovirus or smallpox.

In other embodiments, the infectious agent infects the vaginal mucosa. Such agents include, but are not limited to, human immunodeficiency virus (HIV), human papilloma virus (HPV), herpes simplex virus (HSV) 2, CMV, hepatitis B virus (HBV), and hepatitis C virus (HCV).

In some embodiments, the infectious agent is an enteric virus. As used herein, the term “enteric virus” refers to a virus that infects a cell of the gastrointestinal tract (digestive tract extending from the cavity, through the esophagus, stomach, duodenum, small intestine, large intestine, rectum and anus). Exemplary enteric viruses include but are not limited to coxsackieviruses (type A and B), echoviruses, enteroviruses, and polioviruses. In other embodiments, the infectious agent may be a virus including, but not limited to, herpesvirus, lentivirus, adenovirus, rhinovirus, and rotavirus. In preferred examples, the lentivirus is human immunodeficiency virus.

Respiratory Syncytial Virus

Respiratory syncytial virus (RSV) is the leading cause of serious lower respiratory tract disease in infants and children (Feigen et al., eds., 1987, In: Textbook of Pediatric Infectious Diseases, W B Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season vary by region (Hall, C. B., 1993, Contemp. Pediatr. 10:92-110). In temperate regions of the northern hemisphere, it usually begins in late fall and ends in late spring (Hall, C. B., 1995, In: Mandell G. L., Bernnett J. E., Dolin R., eds., 1995, Principles and Practice of Infections Diseases. 4th ed., Churchill Livingstone, New York at pages 1501-1519). It is estimated that RSV illness results in 90,000 hospitalizations and causes 4,500 deaths annually in the United States. Primary RSV infection occurs most often in children from 6 weeks to 2 years of age and uncommonly in the first 4 weeks of life during nosocomial epidemics (Hall et al., 1979, New Engl. J. Med. 300:393-396). RSV is estimated to cause as much as 75% of all childhood bronchiolitis and up to 40% of all pediatric pneumonias (Cunningham, C. K. et al., 1991, Pediatrics 88:527-532). Children at increased risk from RSV infection include preterm infants (Hall et al., 1979, New Engl. J. Med. 300:393-396) and children with bronchopulmonary dysplasia (Groothuis et al., 1988, Pediatrics 82:199-203), congenital heart disease (MacDonald et al., New Engl. J. Med. 307:397-400), congenital or acquired immunodeficiency (Ogra et al., 1988, Pediatr. Infect. Dis. J. 7:246-249; and Pohl et al., 1992, J. Infect. Dis. 165:166-169), and cystic fibrosis (Abman et al., 1988, J. Pediatr. 113:826-830). The fatality rate in infants with heart or lung disease who are hospitalized with RSV infection is 3%-4% (Navas et al., 1992, J. Pediatr. 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans. Epidemiology and Control, 3rd ed., Plenum Medical Book, New York at pages 525-544). Several epidemics also have been reported among nursing home patients and institutionalized young adults (Falsey, A. R., 1991, Infect. Control Hosp. Epidemiol. 12:602-608; and Garvie et al., 1980, Br. Med. J. 281:1253-1254). Finally, RSV may cause serious disease in immunosuppressed persons, particularly bone marrow transplant patients (Hertz et al., 1989, Medicine 68:269-281).

Treatment options for established RSV disease are limited. Severe RSV disease of the lower respiratory tract often requires considerable supportive care, including administration of humidified oxygen and respiratory assistance (Fields et al., eds, 1990, Fields Virology, 2nd ed., Vol. 1, Raven Press, New York at pages 1045-1072). The only drug approved for treatment of infection is the antiviral agent ribavirin (American Academy of Pediatrics Committee on Infectious Diseases, 1993, Pediatrics 92:501-504). It has been shown to be effective in the treatment of RSV pneumonia and bronchiolitis, modifying the course of severe RSV disease in immunocompetent children (Smith et al., 1991, New Engl. J. Med. 325:24-29). However, ribavirin has a number of limitations including high cost, need for prolonged aerosol administration and potential risk to pregnant women as well as to exposed health care personnel. The American Academy of Pediatrics Committee on Infectious Diseases revised their recommendation for use of ribavirin. The current recommendation is that the decision to use ribavirin should be based on the particular clinical circumstances and physician's experience (American Academy of Pediatrics. Summaries of Infectious Diseases. In: Pickering L. K., ed., 2000 Red Book:Report of the Committee on Infectious Diseases. 25th ed., Elk Grove Village, Ill., American Academy of Pediatrics, 2000, pp. 483-487).

Influenza Viruses

Influenza is an acute febrile illness caused by infection of the respiratory tract. The disease can cause significant systemic symptoms, severe illness requiring hospitalization (such as viral pneumonia), and complications, such as secondary bacterial pneumonia. More than 20 million people died during the pandemic flu season of 1918/1919, the largest pandemic of the 20th century. Recent epidemics in the United States are believed to have resulted in greater than 10,000 (up to 40,000) excess deaths per year and 5,000-10,000 deaths per year in non-epidemic years.

The influenza vaccines currently in use are designated whole virus (WV) vaccine or subvirion (SV) (also called “split” or “purified surface antigen”). The WV vaccine contains intact, inactivated virus, whereas the SV vaccine contains purified virus disrupted with detergents that solubilize the lipid-containing viral envelope, followed by chemical inactivation of residual virus. Attenuated viral vaccines against influenza are also in development. A discussion of methods of preparing conventional vaccine may be found in Wright, P. F. & Webster, R. G., FIELDS VIROLOGY, 4d Ed. (Knipe, D. M. et al. Ed.), 1464-65 (2001), for example.

Parainfluenza Viruses

Human parainfluenza virus type 3 (HPIV3) is a common cause of serious lower respiratory tract infection in infants and children less than one year of age. It is second only to respiratory syncytial virus (RSV) as a leading cause of hospitalization for viral lower respiratory tract disease in this age group (Collins et al., in B. N. Fields Virology, p. 1205-1243, 3rd ed., vol. 1., Knipe et al., eds., Lippincott-Raven Publishers, Philadelphia, 1996; Crowe et al., Vaccine 13:415-421, 1995; Marx et al., J. Infect. Dis. 176:1423-1427, 1997, all incorporated herein by reference). Infections by this virus result in substantial morbidity in children less than 3 years of age. HPIV1 and HPIV2 are the principal etiologic agents of laryngotracheobronchitis (croup) and also can cause severe pneumonia and bronchiolitis (Collins et al., 1996, supra). In a long term study over a 20-year period, HPIV1, HPIV2, and HPIV3 were identified as etiologic agents for 6.0, 3.2, and 11.5%, respectively, of hospitalizations for respiratory tract disease accounting in total for 18% of the hospitalizations, and, for this reason, there is a need for an effective vaccine (Murphy et al., Virus Res. 11:1-15, 1988). The parainfluenza viruses have also been identified in a significant proportion of cases of virally-induced middle ear effusions in children with otitis media (Heikkinen et al., N. Engl. J. Med. 340:260-264, 1999, incorporated herein by reference). Thus, there is a need to produce a vaccine against these viruses that can prevent the serious lower respiratory tract disease and the otitis media that accompanies these HPIV infections. HPIV1, HPIV2, and HPIV3 are distinct serotypes that do not elicit significant cross-protective immunity.

Herpesviruses

The family of herpesviruses is divided into the three subfamilies of alpha-herpesviruses (e.g. herpes simplex virus of type 1 and 2; HSV1 and HSV2), beta-herpesviruses (e.g. cytomegalovirus; HCMV) and gamma-herpesviruses (e.g. Epstein-Barr virus; EBV). The manifestations of infections with herpes viruses are, depending on the virus type, disorders of various organs, such as the skin, the lymphatic system or the central nervous system.

As used herein, the terms “herpes virus” and “herpesvirus” refers to a virus belonging to the Herpesviridae family of large, enveloped double-stranded DNA virus. Exemplary “herpesviruses” include but are not limited to Herpes simplex viruses (HSV1 and HSV2), varicella zoster viruses (VSV), Epstein Barr viruses (EBV), and cytomegaloviruses (CMV).

Infections with the beta-herpesvirus HCMV usually occur during childhood and ordinarily have a subclinical course. The proportion of adults infected worldwide is therefore very high (up to 90%, depending on the investigated population).

Within the family of herpesviruses, cytomegalovirus leads to the highest mortality rate among immunocompromised patients. This is attributable to the fact that cytomegaloviruses cause life-threatening generalized disorders, especially pneumonias, in these people.

The virus particles of herpesviruses have diameters of about 150 to 200 nm and are composed of various structural proteins essential for the virus. The virus core—a fibrillary protein matrix with which the double-stranded linear DNA genome is associated—is located in the interior of the particles. The core is surrounded by an icosahedral capsid which consists of 162 capsomers. The major capsid protein (MCP) of human cytomegalovirus is referred to as UL86.

In certain examples, the herpes virus is selected from the group consisting of Herpes Simplex Type 1, Herpes Simplex Type 2, Cytomegalovirus, Varicella-Zoster virus, Epstein-Barr virus, Human Herpes Virus 6, Human Herpes Virus 7, and Human Herpes Virus 8.

Human Immunodeficiency Virus

HIV infection in humans is considered pandemic. As of January 2006, the Joint United Nations Programme on HIV/AIDS (UNAIDS) and the World Health Organization (WHO) estimate that AIDS has killed more than 25 million people since it was first recognized on Dec. 1, 1981, making it one of the most destructive pandemics in recorded history. It is estimated that about 0.6% of the world's population is infected with HIV (Joint United Nations Programme on HIV/AIDS (2006). “Overview of the global AIDS epidemic”, 2006 Report on the global AIDS epidemic). In 2005 alone, AIDS claimed an estimated 2.4-3.3 million lives, of which more than 570,000 were children.

HIV is a member of the genus Lentivirus, part of the family of Retroviridae. Lentiviruses have many common morphologies and biological properties. Many species are infected by lentiviruses, which are characteristically responsible for long-duration illnesses with a long incubation period (Lévy, J. A. (1993). “HIV pathogenesis and long-term survival”. AIDS 7 (11): 1401-1410). Lentiviruses are transmitted as single-stranded, positive-sense, enveloped RNA viruses. Upon entry of the target cell, the viral RNA genome is converted to double-stranded DNA by a virally encoded reverse transcriptase that is present in the virus particle. This viral DNA is then integrated into the cellular DNA by a virally encoded integrase, along with host cellular co-factors, so that the genome can be transcribed (Smith, Johanna A.; Daniel, Rene (Division of Infectious Diseases, Center for Human Virology, Thomas Jefferson University, Philadelphia) (2006). “Following the path of the virus: the exploitation of host DNA repair mechanisms by retroviruses”. ACS Chem Biol 1 (4): 217-26). Once the virus has infected the cell, two pathways are possible: either the virus becomes latent and the infected cell continues to function, or the virus becomes active and replicates, and a large number of virus particles are liberated that can then infect other cells. Two species of HIV infect humans: HIV-1 and HIV-2.

HIV primarily infects CD4+ T lymphocytes. The HIV replication cycle begins when the glycoprotein envelope gp120 on the surface of the virus links to a CD4 glycoprotein receptor on the host cell. A trans-membrane subunit of the viral membrane, gp41, mediates fusion of the viral membrane and the cell membrane. Once attached, the virus reverse transcribes, integrates, and replicates in a cytolytic fashion, thus shortening the lifespan of the CD4 host cell. Ultimately, viral replication exceeds the immune system's ability to replenish CD4 cells, at which point immunologic function declines and Acquired Immunodeficiency Syndrome (AIDS) develops.

HIV can infect a variety of immune cells, such as CD4+ T cells, macrophages, and microglial cells. HIV-1 entry to macrophages and CD4+ T cells is mediated through interaction of the virion envelope glycoproteins (gp120) with the CD4 molecule on the target cells and also with chemokine coreceptors. Most transmitted strains of HIV-1 use the β-chemokine receptor CCR5 for entry and are thus able to infect and replicate in macrophages and CD4+ T cells (Coakley, E., Petropoulos, C. J. and Whitcomb, J. M. (2005) Curr. Opin. Infect. Dis. 18 (1): 9-15). Macrophages play a key role in several critical aspects of HIV infection, and may be among the first cells infected by HIV. Macrophages and microglial cells are the cells infected by HIV in the central nervous system. The β-chemokines (RANTES, MIP1α, and MIP1β) suppresses replication of these strains of virus by blocking the infection through the CCR5 co-receptor.

Other HIV strains replicate in primary CD4+ T cells as well as in macrophages and use the α-chemokine receptor, CXCR4, for entry. Dual-tropic HIV-1 strains are thought to be transitional strains of the HIV-1 virus and thus are able to use both CCR5 and CXCR4 as co-receptors for viral entry. The α-chemokine, SDF-1, a ligand for CXCR4, suppresses replication of these HIV-1 isolates. It does this by down-regulating the expression of CXCR4 on the surface of these cells. HIV that use only the CCR5 receptor are termed R5, those that only use CXCR4 are termed X4, and those that use both, X4R5. However, the use of co-receptor alone does not explain viral tropism, as not all R5 viruses are able to use CCR5 on macrophages for a productive infection and HIV can also infect a subtype of myeloid dendritic cells (Knight, S. C., Macatonia, S. E. and Patterson, S. (1990). “HIV I infection of dendritic cells”. Int. Rev. Immunol. 6 (2-3): 163-175), which may constitute a reservoir that maintains infection when CD4+ T cell numbers have declined to low levels.

The present invention provides methods of treating infectious disease and/or disorders or symptoms thereof which comprise administering a therapeutically effective amount of a pharmaceutical composition comprising an aerosol immunogenic composition described herein to a subject (e.g., a mammal, such as a human). Thus, one embodiment is a method of treating a subject suffering from or susceptible to an infectious disease or disorder or symptom thereof. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which infection by a pathogen may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a pathogen infection, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Vaccine Production

The invention also provides for a method of inducing an immunological response in a subject, particularly a human, which comprises administering to the subject a viral vector encoding a pathogen polypeptide, or fragment thereof, in a suitable carrier for the purpose of inducing or enhancing an immune response. In one embodiment, an immune response protects the subject from a pathogen infection, such as a herpes, cytomegalovirus, HIV, or AIDs. The administration of this immunological composition may be used either therapeutically in subjects already experiencing a pathogen infection, or may be used prophylactically to prevent a pathogen infection.

If desired, the viral vectors of the invention are combined with a pharmaceutically acceptable carrier, which includes any carrier that does not induce the production of antibodies harmful to the subject receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

Adjuvants are immunostimulating agents that enhance vaccine effectiveness. An adjuvant may be administered in combination with a viral vector of the invention to enhance the effectiveness of an immune response generated against an antigen of interest. The composition may be combined with any other adjuvant known in the art. Effective adjuvants include, but are not limited to, aluminum salts, such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the antigen, pharmaceutically acceptable carrier and adjuvant, also typically contain diluents, such as water, saline, glycerol, ethanol. Auxiliary substances may also be present, such as wetting or emulsifying agents, pH buffering substances, and the like.

Vaccines are administered in a manner compatible with the dose formulation. The immunogenic composition of the vaccine comprises an immunologically effective amount of the virus encoding an antigenic polypeptide and other components. By an immunologically effective amount is meant a single dose, or a vaccine administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgment of the practitioner, but typically range between 5 μg to 250 μg of viral vector per dose.

### Aerosolized Immunogenic Compositions

The invention provides immunogenic compositions that generate an immune response against a pathogen when administered to a subject. In certain examples, the invention features aerosolized immunogenic compositions comprising an immunogenic particle having a size distribution between 0.01 μm and 15 μm. In other examples, the invention features aerosolized immunogenic compositions for inducing a humoral immune response or a cell-mediated immune response in a subject comprising a particle between 0.01 μm and 15 μm in size.

The immunogenic composition, in certain embodiments, is delivered to a mucosal surface. The term “mucosal surface” is meant to refer to any epithelial membrane that covers or lines a body cavity or passage. Mucosal surfaces include, but are not limited to, the respiratory tracts, gastrointestinal tracts, genitourinary tracts, and secretory glands.

In certain specific examples, the mucosal surface is a respiratory surface. A respiratory surface can be any epithelial surface that oxygen or carbon dioxide crosses to move into or out of the body. The mammalian respiratory tract is divided into the upper airways, including the oropharynx, larynx and trachea; the central airways, including the bronchi and bronchioli; and the deep lung, including the alveoli. The lung is the site of many severe, chronic, life-threatening diseases, such as chronic bronchitis, asthma, emphysema, lung cancer, tuberculosis, and other pulmonary infections.

Delivery of various therapeutic agents, particularly macromolecules, to the respiratory tract has proved challenging. Some of the difficulties encountered include excessive loss of inhaled drug in the oropharyngeal cavity, phagocytosis by lung macrophages, and poor control over the site of deposition. Selective delivery into various parts of the respiratory tract by “focal” methods, such as microspray into limited anatomical regions (e.g., nasal or oral cavity, selected airways) has been attempted. Patapoff and Gonda (1997) in “Inhalation Delivery of Therapeutic Peptides and Proteins”, A. Adjei and P. Gupta, eds., Marcel Dekker, Inc. Other methods that have been used include endotracheal catheterization (U.S. Pat. No. 5,803,078). To date, however, no method has been shown to be adequate for the reproducible delivery of polynucleotides to specified portions of the respiratory tract. In addition, delivery to the lung of polynucleotide therapeutics has proved more difficult than delivery of small molecule therapeutics, in part due to the larger size of polynucleotides and their greater susceptibility to physical disruption from the forces required to generate an aerosol, thereby hindering or preventing effective delivery of such polynucleotides.

Accordingly, the invention provides an important improvement because it provides for the effective delivery of polynucleotides to pulmonary tissues. In certain embodiments, the invention features an aerosolized immunogenic composition for generating a humoral immune response or a cell mediated immune response in a subject comprising an immunogenic particle that is between about 0.01 μm and 15 μm in diameter.

Because it is difficult to accurately measure the diameter of small particles, the diameter of one particle of material of a given density will be said to have the same diameter as another particle of the same material if the two particles have the same terminal sedimentation velocity in air under the same conditions. When targeting the deep lung, it is important to have particles that do not have too large a diameter so that the particles can be inhaled deeply into the lungs and thereby deposited on lung tissue and transferred into the patient's circulatory system. It is equally important that such particles not so small that they are inhaled into the lungs and then exhaled without depositing on the lung tissue.

In some embodiments, it may be desirable to target an aerosolized immunogenic composition to particular portions of the lung, such as the large airways of the lung (e.g. the “central airways”) or the alveoli. In other embodiments, it may be desirable to limit delivery to certain portions of the respiratory tract (e.g., the alveoli, the mouth, or the trachea). Selective delivery can be effected by altering particle size. In one embodiment, For example, delivery to the alveoli is used to provide the immunogenic composition to the circulatory system. For alveolar delivery, the immunogenic composition should comprise particle sizes between about 0.1 to about 5 microns. Particles with higher or lower density will effectively behave as bigger or smaller particles, respectively. Similarly, where a subject has a disease affecting lung function, e.g., a disease affecting the small airways and alveoli (e.g., asthma, emphysema, pulmonary infections), it may be desirable to administered smaller particles.

In certain embodiments, the invention features an aerosolized immunogenic composition for generating a cell mediated immune response comprising an immunogenic particle size distribution between 0.01 μm and 15 μm. In other embodiments, the particles range in size between about 10 μm and 15 μm.

Advantageously, the particle size distribution may be tailored to the desired application. For example, in certain embodiments, an immunogenic particle size distribution between 10 μm and 15 μm (e.g., 10, 11, 12, 13, 14, 15) is preferred for generating a cell mediated immune response. This particle size distribution may be used for generating a cell mediated immune response in the absence of a humoral immune response.

In other embodiments, an immunogenic particle size distribution greater than 12 μm is preferred for delivering drugs to a subject's nasal passages. Immunogenic particles below about 5 μm in size (e.g., 1, 2, 3, 4, 4.4, 5 μm) in size are preferred for deep lung and systemic drug delivery using the methods as described herein.

### Prophylactic and Therapeutic Combinations

For some applications, the immunogenic compositions of the invention are provided in combination with other prophylactic or therapeutic agents, or with adjuvants that enhance an immune response. If desired, such additional agents are provided as a submicron suspension, as liposomes, nanosomes, microcapsules or nanocapsules. In certain examples, the one or more additional agents comprise a drug or a combination of drugs for topical or systemic delivery via either a nasal or a pulmonary route. In one embodiment, an immunogenic composition described herein is provided in combination with one or more cytokines, such as interleukins, interferons, tumor necrosis factor (TNF), and colony stimulating factors (GM-CSF, G-CSF, M-CSF). Therapeutic combinations can be delivered by nebulizer (e.g., by nasal or pulmonary delivery) or by any other method known in the art.

### Dosage and Administration

Preferably, the topical concentration is applied to a mucosal lining of the subject, such as in the subject's respiratory tract. Particularly, preferable is the application to the lungs of the subject in the form of an aerosol spray.

In certain examples, for a particular patient population, disease, age, sex and therapeutic or diagnostic polynucleotide, one may need to adjust:

(1) the specific volumes of aerosol and particle free air with consideration to total lung capacity in order to target agent delivery to a specific region of the lungs;

(2) the release point within a patient's inspiratory volume, the release point being as necessary from 0.5 liters or greater up to the patient's vital capacity volume;

(3) the release point within a patient's inspiratory flow rate inside a range of about 0.10 to about 4.0 liters/second preferably about 0.2 to about 3.0 liters per sec.;

(4) particle size for topical pulmonary delivery in a range of about 1-15 microns, 1-5 microns, 0.5 to 5 microns, 2.0 to 4.0 microns, or 10-12 microns;

(5) the amount of heat added to the air to be from 0 Joules to about 100 Joules and preferably less than about 20 Joules to about 50 Joules per 10 μl of formulation;

(6) the relative volume of air added by patient inhalation per 10 μl of formulation is about 100 ml to about 10 liters and preferably about 200 ml to about 5 liters;

(7) the rate of vibration of the porous membrane from 575 to 32,000 kilohertz, preferably 1,000 to 17,000 and more preferably 2,000 to 4,000 kilohertz;

(8) pore size to a range of about 0.25 to about 6.0 microns in diameter preferably 0.5 to 3 microns which is the size of the diameter of the exit opening it being noted that the pore preferably has a conical configuration with the entrance opening being 2 to 20 times the diameter of the exit opening;

(9) viscosity of the formulation to a range of from about 25% to 1,000% of the viscosity of water;

(10) extrusion pressure to a range of about atmospheric-5 psi, 10-20 psi, 25-50 psi, 50 to 1000 psi or 100 to 700 psi;

(11) ambient temperature to 15 C to 30 C and ambient pressure between 1 atmosphere and 75% of 1 atmosphere;

(12) the ratio of bulk media to agent in a formulation to be consistent;

(13) the solubility of agent in bulk media to use highly soluble agents or to use a fine (nanometer size range) dispersion of agent in bulk media;

(14) the desiccator to maximize removal of water, or other carrier, from air;

(15) the shape of the pore opening to be circular in diameter and conical in cross-section with the ratio of the diameter of the small to large end of the cone being about 1/2 to 1/20, and the shape of the porous membrane to an elongated oval;

(16) the thickness of the membrane to 5 to 200 microns; preferably 10 to 50 microns and a tensile strength of over 5,000 psi;

(17) the membrane to have a convex shape or to be flexible so that it protrudes outward in a convex shape preferably beyond the flow boundary layer when formulation is forced through it; and

(18) the firing point to be at substantially the same point at each release for the parameters (1 17), i.e., each release of agent is at substantially the same point so as to obtain repeatability of dosing.

The immunogenic composition is automatically aerosolized at a point in the respiratory cycle after receipt of a signal from a microprocessor programmed to commence aerosol delivery when a signal is received from a monitoring device such as an airflow rate monitoring device. In some applications, a patient using the device withdraws air from a mouthpiece and the total lung capacity, inspiratory flow rate, as well as the inspiratory volume of the patient, are determined one or more times in a monitoring event which determines the volume of aerosol and particle free air to be inhaled and a preferred point in an inhalation cycle for the release of both the aerosol and the particle free air. Inspiratory flow rate and volume, as well as total lung capacity, are each determined and recorded in one or more monitoring events for a given patient in order to develop an inspiratory flow profile for the patient. The recorded information is analyzed by the microprocessor in order to deduce the timing and volume of aerosol and particle free air to be released into the patient's inspiratory cycle with the preferred volumes and point being calculated based on the most likely volume and point to result in repeatably efficient delivery to a specifically targeted area of the lungs.

Pulmonary drug delivery is an attractive alternative to oral, transdermal, and parenteral administration because self-administration is simple, there is no first-pass liver effect of absorbed drugs, and there is reduced enzymatic activity and pH-mediated drug degradation associated with the oral route. Furthermore, structural and physiological features of the lung, including a large mucosal surface and intricate branching for drug absorption, make aerosolization a desirable method for delivering therapeutic agents to the lung.

The preparations are administered in a manner compatible with the dosage formulation, and in such amount as will provide a therapeutic effect.

The methods of the invention can also be used to deliver drugs that cannot be administered orally due to lack of solubility and bio-availability or poor absorption or degradation of drugs in the gastrointestinal tract. Furthermore, the methods of the invention can be used for the systemic administration of drugs which usually require parenteral administration by the intravenous (iv), intramuscular (im), subcutaneous (sc), and intrathecal route.

In certain preferred embodiments, the composition comprises administration of the immunogenic composition in combination with a prime and a boost regimen. Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Vaccine 20:1226-31 (2002); Tanghe, A., Infect. Immun. 69:3041-7 (2001). Providing antigen in different forms in the prime and the boost injections appears to maximize the immune response to the antigen.

In certain cases, the immune response induced by a single dose of a vaccine or immunogenic composition may not be sufficiently strong or sustained to provide effective protection. Repeated administration can enhance the immune response to a vaccine antigen or immunogenic composition, a phenomenon known as “boosting.”

In this approach the immune system is primed by administering an antigen by one method and then boosted by subsequently delivering the same antigen using a different method. Prime-boost vaccination has been shown to stimulate potent cellular immunity by inducing high levels of antigen-specific CD4+ and CD8+ cells. Owing to its ability to elicit a potent cellular immune response, DNA priming followed by adenovirus boosting has been used almost exclusively for vaccinating against viral pathogens. Additionally, there are reports indicating that this approach also stimulates a strong humoral immune response, thus suggesting that this strategy may also be effective for providing protection against bacterial disease (Sullivan, N J et al. (2003). Accelerated vaccination for Ebola virus haemorrhagic fever in non-human primates. Nature 424: 681-684; Tritel, Met al. (2003). Prime-boost vaccination with HIV-1 Gag protein and cytosine phosphate guanosine oligodeoxynucleotide, followed by adenovirus, induces sustained and robust humoral and cellular immune responses. J Immunol 171: 2538-2547).

The prime-boost approach can also be used to enhance the response to potentially convenient vaccines (such as DNA plasmid products) that may not by themselves be sufficiently immunogenic. Additionally, the prime and boost may each stimulate different modalities of the immune response, resulting in a more effective combination of cellular and antibody responses than would be elicited by a single vaccine.

In certain examples, the prime comprises an aerosolized immunogenic particles with a size distribution between 0.01 μm and 15 μm. The boost can comprise a viral vector. In certain examples, the viral vector is an adenoviral vector, for example a recombinant adenoviral vector.

In other certain examples, the prime comprises a viral vector. In certain examples, the viral vector is an adenoviral vector, for example a recombinant adenoviral viral vector. The boost may comprise an aerosolized immunogenic particles with a size distribution between 0.01 μm and 15 μm.

For example, a prime boost regimen may include, but is not limited to DNA-rAd5 (DNA prime, rAd5 encoding an antigen boost); MVA-rAd5; DNA-MVA; protein-rAd5. In particular, since the aerosol route does not produce anti-Ad5 antibodies, a prime-boost regimen might include rAd5-rAd5 where the prime is given by aerosol, and the boost is given either by aerosol or systemically (e.g., by intramuscular, intradermal, or subcutaneous).

Dosing may be carried out as frequently as necessary to maintain therapeutic level, e.g. frequency of dosing may be increased to multiple times a day (e.g. 2, 3, 4, 5, 6, 7, 8, 9, 10, 11, 12, or more times a day) to provide such amount of the immunogenic composition as necessary to maintain therapeutic level or produce an immune response, e.g. therapeutic level in the lung.

One of ordinary skill in the art will be able to readily design effective dosing protocols. An effective dose delivered will usually be in the range of about 1 μg/dosing event to about 500 mg/dosing event, although more or less may be found to be effective depending on various factors, including, but not limited to, the subject's weight, and the desired result. If necessary, dosing can be repeated, based on the subject's response to initial or subsequent dosing. The success of a given dosing event can be measured by various parameters, including, but not limited to, assessment of various physiological parameters, such as, for example, improvement in condition (e.g. therapeutic effect), the presence of any new phenotype which occurs as a result of expression of the delivered polynucleotide.

### Immunogenic Composition Delivery

The invention is based, in part, on the identification of immunogenic compositions comprising polynucleotides that are suitable for delivery as aerosols comprising droplets that are between about 2-15 μm in size. When a vaccine formulation or other immunogenic composition is applied to the device, the resulting aerosol contains the vaccine or immunogenic composition and can deliver it into the lungs by normal breathing. The small sized droplets are dispersed throughout the entire surface of the lung, providing a large and broad delivery of the vaccine formulation or immunogenic composition. Droplets of this size can be produced using any method known in the art. For example, U.S. Pat. No. 4,533,082, discloses a fluid droplet production apparatus with a membrane and a piezo-electric actuator that contracts and expands in order to drive the membrane.

Nebulizers, in particular, inhalation nebulizers that form aerosols are of particular use in the methods of the invention as described herein. In certain examples, the invention uses nebulizers to deliver immunogenic particles. A variety of inhalation nebulizers are known. EP 0170715A1, incorporated by reference in its entirety herein, uses a compressed gas flow to form an aerosol. A nozzle is arranged as an aerosol generator in an atomizer chamber of the inhalation nebulizer and has two suction ducts arranged adjacent a compressed-gas channel. When compressed air flows through the compressed-gas channel, the liquid to be nebulized is drawn in through the suction ducts from a liquid storage container. This nebulizer is an example of continuously operating inhalation nebulizers, in which the aerosol generator produces an aerosol not only during inhalation, but also while the subject exhales. The aerosol produced by the aerosol generator is actually inhaled by the patient only in the inhalation phase, while any aerosol produced at other times is lost. In order to avoid aerosol losses, attempts have been made to restrict aerosol production to part or all of the inhalation phase. Either a patient can interrupt aerosol production manually, or the patient's respiration can be detected by sensors that automatically control aerosol production. Neither situation is flawless, as manual control of aerosol production is an additional strain for patients and often leads to insufficient results. Automatic control of aerosol production represents an enormous technical expenditure which as a rule bears little relation to the obtained benefit.

In the methods of the invention, inhalation nebulizers can be used to deliver therapeutically effective amounts of pharmaceuticals by forming an aerosol which includes particles of a size that can easily be inhaled. The aerosol can be used, for example, by a patient within the bounds of an inhalation therapy, whereby the therapeutically effective pharmaceutical or drug reaches the patient's respiratory tract upon inhalation.

One such inhalation nebulizer is the PARI device, which is commercially available. PARI's eFlow, an electronic, portable nebulizer, enables aerosolization of liquid medications via a vibrating, perforated membrane. The PARI device is described in U.S. Pat. No. 5,152,456, U.S. Pat. No. 5,261,601, U.S. Pat. No. 5,518,179, U.S. Pat. No. 6,962,151, incorporated by reference in their entireties herein. Information on the PARI device can be found publicly on the world wide web at http://www.paripharma.com/technologiesl.htm.

U.S. Pat. No. 5,152,456, incorporated by reference in its entirety herein, describes a dispensing apparatus that comprises a housing defining a chamber receiving liquid to be dispensed and comprising a perforate membrane which defines a front wall of the chamber. A vibrating device is connected to the housing and is operable to vibrate the perforate membrane to dispense droplets of liquid through holes in the perforate membrane. The housing comprises an annular member having a relatively thin inner annular portion connected to the perforate membrane and a relatively thick outer annular portion connected to the vibrating device. The U.S. Pat. No. 5,152,456 patent describes an apparatus that is suitable for dispensing pharmaceutical products as an atomized mist, and provides a hand-held inhaler for oral inhalation.

U.S. Pat. No. 5,261,601, incorporated by reference in its entirety herein, describes a dispensing apparatus for use in dispensing liquid as an atomized spray. The U.S. Pat. No. 5,261,601 describes a dispensing apparatus that comprises a housing defining a chamber receiving liquid to be dispensed and comprising a perforate membrane which defines a front wall of the chamber. A vibrating means is connected to the housing and is operable to vibrate the perforate membrane to dispense droplets of liquid through holes in the perforate membrane. The membrane defines an array of holes each of which is flared such that the cross-section narrows in a direction from the rear surface of the membrane in contact with the liquid towards the front surface of the membrane. The apparatus is suitable for dispensing pharmaceutical products as an atomized mist and provides a hand-held inhaler for oral inhalation.

U.S. Pat. No. 5,518,179, incorporated by reference in its entirety herein, describes a fluid droplet production apparatus, for example, for use an atomizer spraying device, that has a membrane which is vibrated by an actuator, which has a composite thin-walled structure and is arranged to operate in a bending mode. According to a certain embodiments of the invention the fluid droplet apparatus comprises a membrane, an actuator, for vibrating the membrane, where the actuator comprises a composite thin-walled structure arranged to operate in a bending mode and to vibrate the membrane substantially in the direction of actuator bending, and a means for supplying fluid directly to a surface of the membrane, as fluid is sprayed therefrom on vibration of the membrane. The membrane is structured so as to influence the menisci of fluid introduced to the membrane.

In certain preferred embodiments, the actuator is substantially planar, but it is possible that thin-walled curved structures may be appropriate in some circumstances. Another thin-walled structure which is not planar, would be a structure having bonded layers in which the stiffness of each layer varied across the common face area over which they are bonded in substantially the same way. In all cases, the actuator is thin-walled over its whole area. Fluid is brought from a fluid source directly into contact with the membrane (which may be tapered in thickness and/or have a textured surface) and is dispensed from the membrane by the operation of the vibration means, (advantageously without the use of a housing defining a chamber of which the membrane is a part). The membrane may be a perforate membrane, in which case the front face may have annular locally raised regions disposed substantially concentrically with the holes.

Preferably, the holes defined by a perforate membrane each have a relatively smaller cross-sectional area at the front face and a relatively larger cross-sectional area at the rear face, and can be referred to as tapered holes. In certain examples, the reduction in cross-sectional area of the tapered holes from rear face to front face is smooth and monotonic. Such tapered holes are believed to enhance the dispensation of droplets. In response to the displacement of the relatively large cross-sectional area of each hole at the rear face of the perforate membrane a relatively large fluid volume is swept in this region of fluid.

The size of the smaller cross-sectional area of the perforations on the front face of the membrane may be chosen in accordance with the diameter of the droplets desired to be emergent from the membrane. Dependent upon fluid properties and the excitation operating conditions of the membrane, for circular cross-sectional perforation the diameter of the emergent droplet is typically in the range of 1 to 3 times the diameter of the perforation on the droplet-emergent face of the membrane.

Other factors, such as the exact geometrical form of the perforations, being fixed, the degree of taper influences the amplitude of vibration of the membrane needed for satisfactory droplet production from that perforation. Substantial reductions in the required membrane vibrational amplitude are found when the mean semi-angle of the taper is in the range 30 degrees to 70 degrees, although improvements can be obtained outside this range.

For perforate membranes with tapered perforations as described above, it is found that fluid may be fed from the fluid source by capillary feed to a part of the front face of the membrane and in this embodiment fluid is drawn through at least some of the holes in the membrane to reach the rear face of the membrane prior to emission as droplets by the action of the vibration of the membrane by the vibration means. This embodiment has the advantage that, in dispensing fluids that are a multi-phase mixture of liquid(s) and solid particulate components, examples being suspensions and colloids, only those particulates whose size is small enough in comparison to the size of the holes for their subsequent ejection within fluid droplets pass through from the front to the rear face of the perforate membrane. In this way the probability of perforate membrane clogging by particulates is greatly reduced.

The faces of the membrane need not be planar. In particular, for perforate membranes, the front face may advantageously have locally raised regions immediately surrounding each hole. Such locally-raised regions are believed to enhance the dispensation of droplets by more effectively “pinning” the menisci of the fluid adjacent to the front face of the holes than is achieved by the intersection of the holes with a planar front face of the membrane, and thereby to alleviate problems with droplet dispensation caused by “wetting” of the front face of the membrane by the fluid.

Other conditions being fixed, such tapered perforations reduce the amplitude of vibration of the perforated membrane needed to produce droplets of a given size. One reason for such reduction of amplitude being achieved is the reduction of viscous drag upon the liquid as it passes through the perforations. Consequently a lower excitation of the electromechanical actuator may be used. This gives the benefit of improved power efficiency in droplet creation.

One advantage of the arrangement of the invention is that a relatively simple and low cost apparatus may be used for production of a fluid droplet spray.

U.S. Pat. No. 6,962,151, incorporated by reference in its entirety herein, describes an inhalation nebulizer having both an aerosol generator and a mixing chamber. The aerosol generator includes a liquid storage container for a liquid medicament. In this, a liquid medicament can be a drug that is itself a liquid, or the liquid medicament can be a solution, suspension or emulsion that contains the medicament of interest. In a preferred embodiment, the liquid medicament is an active agent that is in a solution, a suspension or an emulsion.

The aerosol generator also includes a diaphragm that is connected on one side to the liquid storage container, such that a liquid contained in the liquid storage container will come into contact with one side of the diaphragm. The diaphragm is connected to a vibration generator that can vibrate the diaphragm so that a liquid in the liquid storage container can be dispensed or dosed for atomization through openings present in the diaphragm and enter the mixing chamber.

The mixing chamber has an inhalation valve that allows ambient air to flow into the mixing chamber during an inhalation phase while preventing aerosol from escaping during an exhalation phase. The mixing chamber also has an exhalation valve that allows discharge of the patient's respiratory air during the exhalation phase while preventing an inflow of ambient air during the inhalation phase.

The aerosol generator may include a cylindrical liquid storage container that is defined on one side by a diaphragm that preferably is a circular disk. A liquid filled in the liquid storage container contacts one side of the diaphragm. A vibration generator, for instance a piezoelectric crystal, surrounds the diaphragm circumferentially such that the diaphragm can be vibrated by the vibration generator. This requires a electric drive circuit for the vibration generator, the structure and function of which are well known to the person skilled in this art. Through openings present in the diaphragm, the liquid adjoining one side of the diaphragm is atomized through to the other side of the diaphragm and thus is atomized into the mixing chamber.

The liquid storage container preferably provides an entry point for the medicament to be dispensed. In one embodiment, the liquid storage container is a liquid reservoir that is directly fitted into the inhalation nebulizer. In another embodiment, the medicament is provided to the liquid storage container as a metered volume from either a single dose or multi dose container. If a multi dose container is used, it is preferably equipped with a standard metering pump system as used in commercial nasal spray products. If the liquid storage container is cylindrical, it is preferred that the diaphragm has a circular design and the vibration generator has an annular design. The inhalation nebulizer includes an aerosol generator and a mixing chamber having an inhalation valve and an exhalation valve.

Preferably, the aerosol generator is arranged in a section of the mixing chamber that is also of a cylindrical design. Thereby an annular gap is obtained around the aerosol generator through which the ambient air can flow into the mixing chamber during the inhalation phase.

A mouthpiece is preferably integrally formed with the mixing chamber, but it also can be attached removably to the mixing chamber. A subject inhales the aerosol through the mouthpiece. The aerosol is generated by the aerosol generator and is stored in the mixing chamber. The size and the form of the mouthpiece can be chosen such that it enlarges the mixing chamber and simultaneously provides for the arrangement of the exhalation valve. The exhalation valve is preferably located adjacent the opening of the mouthpiece facing the subject.

When a subject exhales into the opening of the mouthpiece, the exhalation valve is opened so that the respiratory air of the subject is discharged into the surroundings. To this end, a valve element of the exhalation valve is lifted and frees the opening of the exhalation valve. The inhalation valve is closed when the subject exhales into the inhalation nebulizer, as the valve element of the inhalation valve closes the opening of said valve. When a subject inhales through the opening of the mouthpiece, the inhalation valve is opened and frees the opening as the valve element is lifted. Thereby ambient air flows through the inhalation valve and the annular gap into the mixing chamber and is inhaled by the subject together with the aerosol. As aerosol has accumulated in the mixing chamber during an exhalation phase, there is available to the subject an increased amount of aerosol, a so-called aerosol bolus, especially at the beginning of an inhalation phase.

Aerosols of the invention may be dispersed by jet, ultrasonic nebulizer, or electronic nebulizer. Alternatively, the formulation may be administered as a dry powder using a metered dose inhaler or a dry powder inhaler, for example. Aerosolized formulations deliver high concentrations of vaccine directly to airways with low systemic absorption, and include for example, inhalation solutions, inhalation suspensions, and inhalation sprays. Inhalation solutions and suspensions are aqueous-based formulations containing the genetic vaccine and, if necessary, additional excipients. Such formulations are intended for delivery to the respiratory airways by inspiration.

One factor to be considered in pulmonary delivery is reaching the deep lung. To achieve high concentrations of a genetic vaccine in both the upper and lower respiratory airways, the vaccine is preferably nebulized in jet nebulizers, an ultrasonic nebulizer, or an electronic nebulizer particularly those modified with the addition of one-way flow valves, such as for example, the Pari LC PIus™ nebulizer, commercially available from Pari Respiratory Equipment, Inc., Richmond, Va., which delivers up to 20% more drug than other unmodified nebulizers.

The pH of the formulation is also important for aerosol delivery. When the aerosol is acidic or basic, it can cause bronchospasm and cough. A comfortable range of pH depends on a patient's tolerance. An aerosol solution having a pH between 5.5 and 7.0 is usually considered tolerable. To avoid bronchospasm, the pH of the formulation is preferably maintained between 5.5 and 7.0, most preferably between 5.5 and 6.5 to permit generation of a vaccine aerosol well tolerated by patients without any secondary undesirable side effects, such as bronchospasm and cough. Propellants, such as HFA 134a, HFA 227, or combinations thereof, may also be used in the formulation. If desired, excipients that promote drug dispersion or enhance valve lubrication may also be formulated with the vaccine.

In certain embodiments of the invention, the particles produced by aerosolization, for example using the nebulizer devices, are 0.01-5 um in size. In certain embodiments of the invention, the particles produced by aerosolization, for example using the nebulizer devices, are 2-5 μm in size. In other certain embodiments of the invention, the particles produced by aerosolization, for example using the nebulizer devices, are 10-15 μm in size.

In certain examples, the specified area of the respiratory tract to which the particles are delivered is determined by the particle size. For instance, a particle of 0.01-5 um in size, e.g. a 0.01, 0.1, 0.5, 1, 1.1, 1.2, 1.5, 2, 2.1, 2.2, 2.5, 3, 3.1, 3.2, 3.5, 4, 4.5, or 5 micron sized particle may be delivered to the whole lung. In other embodiments, a particle of a size of 10-12 um, for example 10, 10.5, 11, 11.5, 12 micron may be delivered to the large upper airways. The location of delivery of the particles may, in certain embodiments, have an effect on immune response generated, for example, humoral or cellular immune response. In certain examples, the immune response generated by delivery of the immunogenic composition is determined by the particle size, as described below.

In certain examples, the invention features methods for delivering an immunogenic composition to one or more mucosal surfaces of a subject. The mucosal surface can be a distal surface, selected from, but not limited to, a pulmonary airway, gastrointestinal surface, genitourinary surface, and secretory gland. The mucosal surface, in other certain examples, is a respiratory surface.

An aspect of the invention involves manipulating the particle size in order to treat (target) particular areas of the respiratory tract. By creating aerosolized particles which have a relatively narrow range of size, it is possible to further increase the efficiency of the agent delivery and improve the reproducibility of the dosing. Aerosol particle size can be adjusted by adjusting the size of the pores of the membrane. In general, for delivery to the respiratory tract, the aerosol is created by forcing the drug formulation through a nozzle comprised of a porous membrane having pores in the range of about, but not limited to, 2 to 15 microns in size. When the pores have this size the droplets that are formed will have a diameter approximately equal to the pore size, depending on factors such as the viscosity of the fluid. In order to ensure that the low resistance filter has the same or less flow resistance as the nozzle, the pore size and pore density of the filter should be adjusted as necessary with adjustments in pore size and pore density of the nozzle's porous membrane.

Particle size can also be adjusted by the use of a vibration device which provides, for example, a vibration frequency in the range of about 800 to about 4000 kilohertz. Vibration devices useful in the delivery devices of the present invention are described in U.S. Pat. Nos. 5,497,763; 5,819,726; 5,906,202; and 5,522,385, each of which is incorporated herein by reference in their entireties.

### Dry Powder Formulation

As an alternative therapy to aerosol delivery, vaccines of the invention are administered in a dry powder formulation for efficacious delivery into the endobronchial space. Such formulations have several advantages, including product and formulation stability, high drug volume delivery per puff, and low susceptibility to microbial growth. Therefore, dry powder inhalation and metered dose inhalation are most practical when high amounts of vaccine need to be delivered. Depending on the efficiency of the dry powder delivery device, effective dry powder dosage levels typically fall in the range of about 20 to about 60 mg. The invention therefore provides a sufficiently potent formulation of a genetic vaccine in dry powder or metered dose form of drug particles. Such a formulation is convenient because it does not require any further handling such as diluting the dry powder. Furthermore, it utilizes devices that are sufficiently small, fully portable and tend to have a long shelf life.

For dry powder formulations of the invention, a genetic vaccine composition is milled to a powder having mass median aerodynamic diameters ranging from 1-15 microns by media milling, jet milling, spray drying, super-critical fluid energy, or particle precipitation techniques. Particle size determinations may be made using a multi-stage Anderson cascade impactor or other suitable method. Alternatively, the dry powder formulation may be prepared by spray drying or solution precipitation techniques. Spray drying has the advantage of being the least prone to degrading the vaccine. Solution precipitation is performed by adding a co-solvent that decreases the solubility of a drug to a uniform drug solution. When sufficient co-solvent is added the solubility of the drug falls to the point where solid drug particles are formed which can be collected by filtration or centrifugation. Precipitation has the advantage of being highly reproducible and can be performed under low temperature conditions, which reduce degradation. Super-critical fluid technology can produce particles of pharmaceutical compounds with the controlled size, density and crystallinity ideal for powder formulations.

The dry powder formulations of the present invention may be used directly in metered dose or dry powder inhalers. Currently, metered dose inhaler technology is optimized to deliver masses of 1 microgram to 5 mg of a therapeutic. Spacer technology, such as the aerochamber, may also be utilized to enhance pulmonary exposure and to assist patient coordination.

An alternate route of dry powder delivery is by dry powder inhalers. There are two major designs of dry powder inhalers, device-metering designs in which a reservoir of drug is stored within the device and the patient “loads” a dose of the device into the inhalation chamber, and the inspiratory flow of the patient accelerates the powder out of the device and into the oral cavity. Alternatively, dry powder inhalers may also employ an air source, a gas source, or electrostatics, in order to deliver the vaccine. Current technology for dry powder inhalers is such that payload limits are around 10 mg of powder. The dry powder formulations are temperature stable and have a physiologically acceptable pH of 4.0-7.5, preferably 6.5 to 7.0.

In other embodiments, a genetic vaccine is administered as a liquid aerosol. In solution, the concentration of the vaccine is about 0.5, 1, 5, 10, 20, 40, 60, 80, 100 mg/mL, or more and is formulated in a physiologically acceptable solution, preferably in one quarter strength of normal saline. Ideally, the subject is administered with at least 10, 50, 100, 200, 500, 700, 1000, or more than 1000 micrograms of a genetic vaccine administered as an aerosol. The use of dry powder inhalation preferably results in the delivery of at least about 1, 5, 10, 20, 30, 40, 50, 60, or more than 60 mg of the genetic vaccine to the respiratory airways of the patient receiving treatment. In such a formulation, the genetic vaccine is delivered as a powder in an amorphous or crystalline state in particle sizes between 1 and 15 μm in mass median aerodynamic diameter necessary for efficacious delivery of the genetic vaccine into the endobronchial space for treatment, amelioration, or prevention of a pathogen infection. Fractions of 2 to 4 microns may also be employed to target the peripheral lung. Patient inspiration techniques, such as breath holding for example, may also optimize deposition of the vaccine.

In other embodiments, the invention features a device comprising a genetic vaccine, one or more propellants, and if desired, a surfactant. The liquefied propellant serves as an energy source to expel the formulation from the valve in the form of an aerosol, and as a dispersion medium for the drug and surfactant. The surfactant lubricates the metering valve mechanism, and helps disperse drug particles. Drug dissolution usually necessitates the addition of less volatile ethanol. In one embodiment, the device is a metered-dose inhaler (MDI). Typically, an MDI is provided a molded plastic actuator which positions the subject's lips very close to the spray orifice. Optionally, it may also be provided with a spacer, which is a hollow tube that provides for enhanced delivery of the vaccine.

### Kits

The invention provides kits for the treatment or prevention of pathogen infection. In one embodiment, the kit includes a therapeutic or prophylactic composition containing an effective amount of an immunogenic composition in unit dosage form. In some embodiments, the kit comprises a device (e.g., nebulizer, metered-dose inhaler) for immunogenic composition dispersal or a sterile container which contains a therapeutic or prophylactic immunogenic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an immunogenic composition of the invention is provided together with instructions for administering the immunogenic composition to a subject having or at risk of developing an infectious disease (e.g., tuberculosis, influenza, RSV, HIV). The instructions will generally include information about the use of the composition for the treatment or prevention of a pathogen infection. In other embodiments, the instructions include at least one of the following: description of the therapeutic agent; dosage schedule and administration for treatment or prevention of pathogen infection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Aerosolized rAd5 Encoding an Antigen Induced IL2 Production and Polyfunctional T Cells

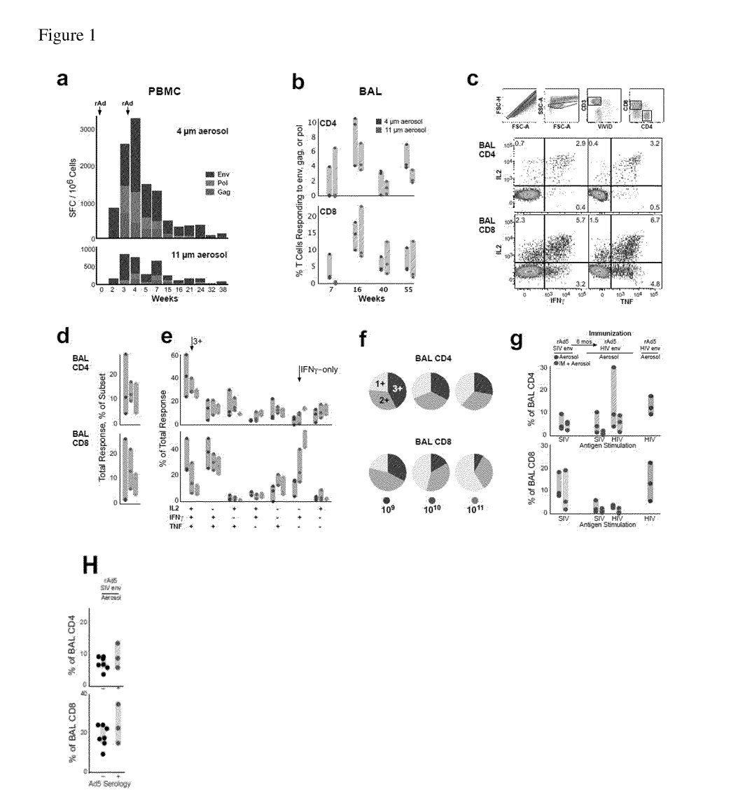

The qualitative and quantitative aspects of immune responses elicited by immunization with replication deficient, recombinant adenoviral serotypes 5 (rAd5) and 35 (rAd35) encoding antigens delivered to the lung in defined particle sizes using an Investigational eFlow Nebulizer System were studied. To determine if this approach could generate local, systemic, and mucosal adaptive responses, six rhesus macaques were immunized in a pilot experiment. For each animal, a mixture of 10¹¹ particles of rAd5 encoding SIV env⁹ and 10¹¹ particles encoding an SIV Gag-Pol fusion protein (FIG. 8) were administered by aerosol. Two different drop sizes were used to test whether immunogenicity was affected by delivery to the upper airways (11 μm aerosol) vs. the entire lung (4 μm). Animals were immunized twice with a three week interval. T cell responses in PBMC measured by IFNγ ELISpot peaked at week four, one week after the second immunization (FIG. 1A); the 4 μm aerosol generated about 3-fold greater responses than the 11 μm aerosol. Thereafter, the response declined to background levels by 3-6 months. Immune complexes (ICS) measurements showed a similar kinetic, with the peak reaching 0.5 to 1% of CD4 or CD8 memory cells producing cytokines in response to in vitro SIV peptide stimulation, and then becoming undetectable over the next few months. There were no measurable responses in jejunal biopsies or inguinal or mesenteric lymph node biopsies. The hierarchy of responses to the antigens encoded by the inserts is the same as has been reported for intramuscular immunization: Env generated the largest responses, followed by Pol and Gag.

In contrast to the transient nature of the T cell responses from peripheral blood monocytes, T cell responses measured in the bronchoalveolar lavage (BAL) were substantially higher and much more durable (FIG. 1B). Unlike the systemic T cell responses, there was no difference in the magnitude of the responses induced by 4 μm vs. 11 μm aerosols in the BAL.

An important facet of a vaccine-induced T cell response is its quality: the repertoire of functions that are elicited. Aerosol immunization induced CD4 and CD8 T cell responses in the BAL that were highly polyfunctional (simultaneously making IFNγ, TNF, and IL2; see, e.g., FIG. 1C). This is unlike peripheral T cells induced by intramuscular rAd5, for which responses are dominated by IFNγ-only cells. The production of IL2 and the polyfunctional nature of the cells are highly desirable from the standpoint of generating persistent, highly effective T cells. The polyfunctional T cells induced by the aerosolized vaccination also secreted much larger amounts of cytokine on a per-cell basis (as estimated by the median fluorescence intensity), as has been previously described for optimized polyfunctional effector T cells (Darrah, et al., Nat Med 13 (7), 843 (2007)).

To determine the dose response to immunization with rAd5 encoding an antigen by this route, three macaques were immunized. Each animal received rAd5 encoding HIV envA (Seaman et al., J Virol 79 (5), 2956 (2005)) with doses ranging from 10⁹ to 10¹¹ particles. Over this range, the total magnitude of the T cell response in the BAL was not demonstrably affected (FIG. 1D); however, differences in the quality of the T cell response were detected (FIGS. 1E, F). As has been demonstrated for immunization with rAd5 encoding an antigen in mice, higher doses of rAd5 are associated with less polyfunctional responses (Darrah, et al., Nat Med 13 (7), 843 (2007)). In particular, the high dose immunization shifted the balance of CD4 and CD8 T cells from a polyfunctional response toward one comprised of only IFNγ.

### Example 2

### Potent Immunogenicity Identified in Seropositive Animals.

While rAd5 vectors elicit potent T cell responses in humans, a major limitation is their decreased immunogenicity when given intramuscularly (IM) to individuals who are seropositive against Ad5 from prior exposure to infection (Barouch and Nabel Hum Gene Ther 16 (2), 149 (2005)). To determine the effect of pre-existing Ad5 immunity on the immunogenicity of rAd5 encoding an antigen given by aerosol, macaques were successively immunized with different rAd5 vectors. First, three animals were each given either 10¹¹ rAd5 SIV env solely by aerosol, or simultaneously by aerosol and IM. The BAL T cell response to SIV env following this immunization was robust (FIG. 1G, left). The animals generated either low or high levels of neutralizing antibodies to Ad5 in their serum (Table 1, below).

TABLE 1
Induction of anti-vector antibodies

| First Regimen | | | | | Second Regimen⁴ | |
|---|---|---|---|---|---|---|
| | | Drop | # of | | Route and | |
| Route¹ | Dose | size² | times³ | Ad5 IC90⁴ | Dose | Ad5 IC90⁶ |
| AE | 10¹¹ | 4 μm | 2 | 432⁵, 1249, 1144 | AE 10¹⁰ | 322 |
| AE | 10¹¹ | 11 | 2 | nd⁵, nd⁵, nd | AE 10¹⁰ | nd, nd |
| AE | 10⁹ | 4 | 1 | nd, nd, nd | | |
| AE | 10¹⁰ | 4 | 1 | nd, nd, nd | | |
| AE | 10¹¹ | 4 | 1 | 44, nd, nd | | |
| IM + AE | 10¹⁰ | 4 | 1 | >8800⁵, >8800⁵, >8800⁵ | AE 10¹⁰ | >8800, >8800, >8800 |

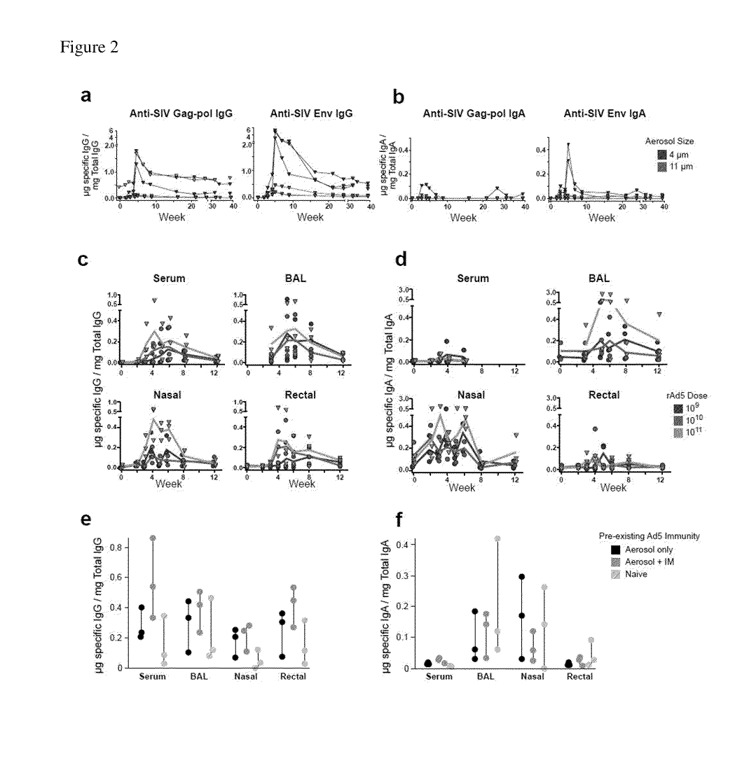

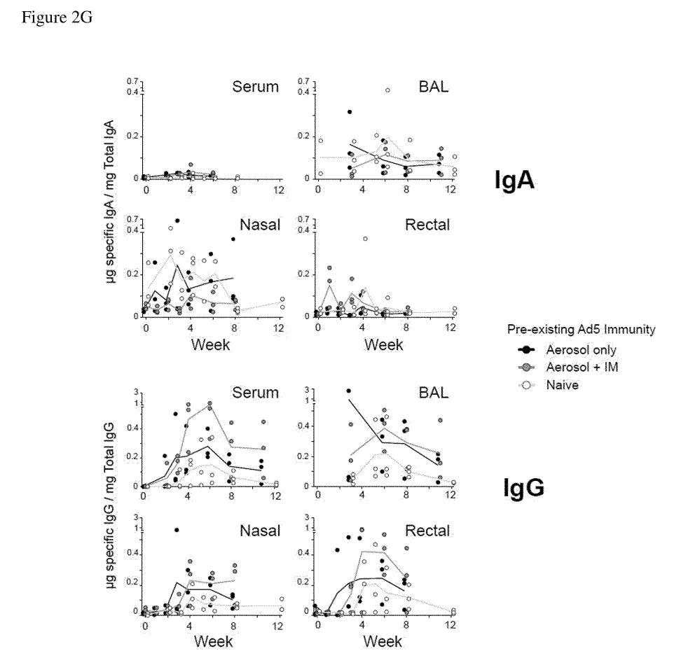

Notes:
¹AE = Aerosol, IM = intramuscular. For each group, 3 animals were immunized.
²See materials and methods.
³Number of administrations (spaced at 3 week intervals)
⁴Serum dilution that inhibits 90% of Ad5 infectivity, measured 8-40 weeks after the primary immunizations, nd = below the limit of detection, <1:12.
⁵Some animals were immunized with another regimen 6-14 months after the first immunization. All immunizations in the second regimen were single administrations using a 4 μm aerosol drop size. Immunogenicity for these 6 animals (and 3 Ad-naïve animals) is reported in FIGS. 1g and 2e, f.
⁶IC90 was measured 8 weeks after the second regimen.

Over 6 months later, these animals were then immunized with 10¹⁰ particles of rAd5 encoding a different antigen, HIV envA. This vector induces strong T cell responses in the BAL when given by aerosol to Ad5-naïve animals (FIG. 1G, right). All six pre-immunized animals generated very strong CD4 T cell responses to this second immunogen, even the three with very high levels of neutralizing Ad5 antibodies (FIG. 1G, middle). The CD8 responses were decreased in animals previously exposed to Ad5; however, the responses were still detectable (1-5% of CD8 T cells), and higher than achieved with other vaccination routes. Notably, the second rAd5 immunization did not eliminate the T cell responses to the original immunization (SIV env), showing that the responses generated after the initial immunization persist through additional heterologous immunizations.

In a separate experiment, we immunized 10 naïve animals with aerosolized rAd5 encoding SIV antigens; of these animals, three were Ad5 seropositive from previously acquired (natural) infection. The BAL CD4 and CD8 T cell responses following immunization of the seropositive animals were no lower than those in the seronegative animals (FIG. 1h), indicating that natural infection with Ad5 does not limit the immunogenicity of aerosolized recombinant Ad5 vectors.

Thus, aerosolized immunization with aerosolized rAd5 encoding an antigen elicited strong peripheral cellular responses that peaked within 4-6 weeks and subsequently decreased to background levels, while the BAL responses were very strong and durable. The quality of the T cell response was best at lower doses of rAd5 encoding an antigen, and the ability to generate de novo T cell responses in the BAL was not strongly affected by previous exposure to Ad5 given systemically or by aerosol.

### Example 3

### Aerosolized rAd5 Encoded Antigens Elicited Robust Humoral Responses

In addition to T cells, induction of antibody responses at mucosal surfaces is important for mediating protection against many viral and bacterial infections. Thus, the induction of antibodies to both the antigens encoded by the gene inserts as well as the rAd5 vector used in these immunizations was determined locally and systemically. Aerosol immunization generated very poor anti-vector (anti-Ad5) immune responses in the serum at doses of 10⁹-10¹¹ (Table 1). Measurable neutralizing titres were only observed following repeated immunization with the highest dose (10¹¹ particles), and these were still dramatically lower than responses observed following IM alone or IM+aerosol (AE) immunization. However, aerosolized rAd5 elicited robust humoral responses to the antigens encoded by the gene inserts. Serum IgG levels peaked ˜6 weeks after immunization and persisted for at least 6 months (FIG. 2A). IgA levels in the serum were also measurable, albeit at much lower levels (FIG. 2B). As was seen for anti-vector humoral responses (Table 1) and the transient systemic cellular responses (FIG. 1A), the 4 μm drop size generated stronger humoral responses to the antigens encoded by the gene inserts than did the 11 μm aerosol.

Specific humoral responses were generated not only systemically, but mucosally, at both local and distal sites. In the dose-response study, higher responses were attained at the highest dose, although even the lowest dose was immunogenic (FIG. 2C, D), inducing both IgG and IgA. Importantly, the specific IgA levels at the local mucosa were much higher than in the serum—indicative that the IgA was locally-produced secretory IgA and not solely transudated serum IgA. Finally, the question of whether humoral immunogenicity was affected by the presence of neutralizing anti-vector antibodies was addressed. Similar to the cellular responses (FIG. 1G), aerosolized rAd5 generated equivalent humoral responses irrespective of the pre-existing levels of anti-Ad5 antibodies (FIG. 2E,F). Thus, immunization with aerosolized rAd5 encoding an antigen is effective at generating humoral responses when delivered by 4 μm drop size. Moreover, this mode of immunization resulted in secretory IgA at local mucosal sites.

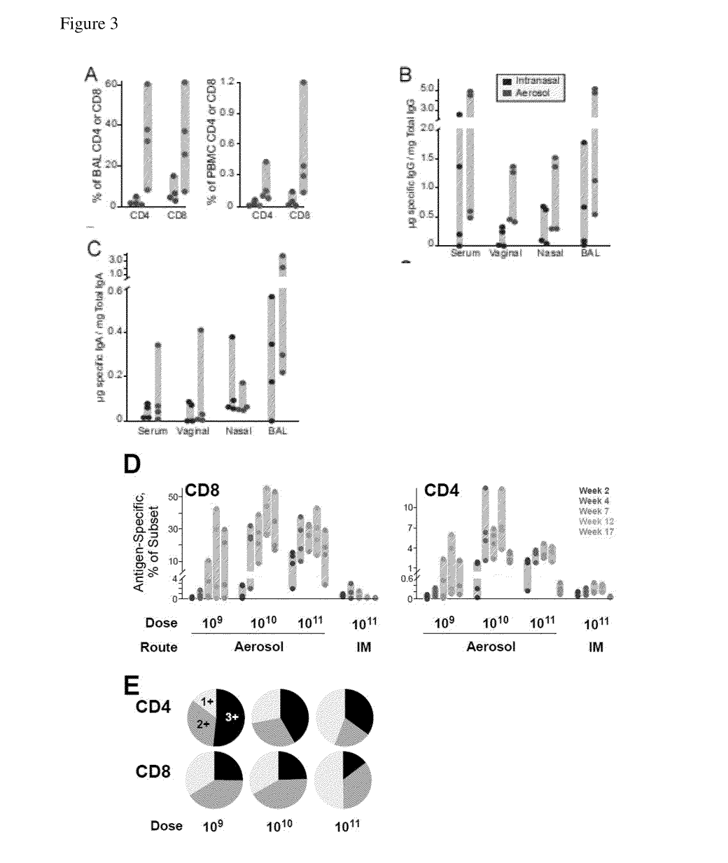

Interestingly, the cellular and humoral immunogenicity profiles observed after delivery of rAd to the lung appear unique. Intranasal (IN) delivery of vaccines has been studied for many years and is clinically approved for influenza vaccination. To compare and contrast aerosol delivery to intranasal delivery, we inoculated eight rhesus macaques in two groups, four receiving 10¹⁰ particles of rAd5 encoding HIV envelope by 4 μm aerosol, and four receiving the same dose intranasally (FIG. 3). Animals were immunized twice, four weeks apart. Results from the aerosol delivery were consistent with earlier studies (FIGS. 1 and 2).

Intranasal delivery generated weak but detectable cellular responses in the BAL and the PBMC (FIG. 3A): both CD4 and CD8 T cells specific to the HIV env were quantified. The aerosol delivery elicited almost 10-fold greater responses than IN for both CD4 and CD8. The quality of the T cell response was similar for CD8 cells; for CD4, the aerosol elicited a greater proportion of the more-differentiated IFNγ-producing cells, whereas IN elicited a greater proportion of TNF-producing cells. This demonstrates that the aerosol delivery provides a much stronger induction of cellular responses than does intransal delivery.

Induction of antigen-specific IgG (FIG. 3B) and IgA (FIG. 3C) was similar between the two routes, although there was a trend towards greater induction by the aerosol route than the IN route. Of note, aerosol immunization induced 4-fold higher vaginal IgG responses than IN; however, these were in concordance with the higher serum levels and may simply reflect transudation of serum immunoglobulin. Importantly, both IN and aerosol delivery generated high levels of IgA in the respiratory mucosa, exceeding that in serum, indicative of secretory IgA at this site.

### Example 4

### Immunogenicity of Aerosolized rAd35 Encoding TB Antigens

The generation of large T cell responses in the lung may be highly desirable for containing respiratory pathogens, such as TB, RSV, and influenza. To test this concept, a pilot immunization of rhesus macaques was performed using a rAd35 vector encoding the TB antigens 85A/B and 10.4 (Radosevic, K. et al., Infect Immun 75 (8), 4105 (2007)) (FIG. 10, 11). rAd35 encoding TB antigens 85A/B and 10.4 was highly immunogenic in the lungs when given by aerosol to rhesus macaques (FIG. 3D). A single dose of even 10⁹ particles of rAd35 encoding an antigen generated measurable T cell responses; a second dose boosted the response in most animals. Higher doses gave very strong T cell responses; responses ranged from 25-50% of CD8 T cells and from 3-12% of CD4 T cells. Notably, even two IM immunizations with 10¹¹ particles generated BAL responses that were 10-50 fold lower than the aerosol regimen.

In this experiment, three of the animals across the dose groups were Ad35 seropositive from previous (naturally acquired) infection. However, consistent with the data in FIG. 1G, there was no decrease in the BAL T cell responses in these animals compared to the seronegative animals. Finally, as was seen for rAd5 encoding an antigen, the quality of the T cell response varied with dose, with the lower doses of rAd35-based vaccines giving more polyfunctional T cell responses (FIG. 3E).

### Example 5

### Aerosolized rAd5 Encoding Influenza Hemagglutinin (HA) Protected against Influenza

To determine whether aerosolized rAd5-based vaccines could protect against challenge with a respiratory pathogen, ferrets were immunized with rAd5 encoding influenza hemagglutinin (HA) or nucleoprotein (NP) (Indonesia strain) by 4 μm aerosol. rAd5 has been demonstrated to afford protection after IM immunization (Gao, W et al. (2006) J. Virol 80(4):1959-64; Hoelscher, M. A. et al. (2006) Lance 367(9509):475-81). Eighteen days after immunization, the animals were challenged intranasally with a near-lethal dose of H5N1 avian influenza (Vietnam 1203/04).

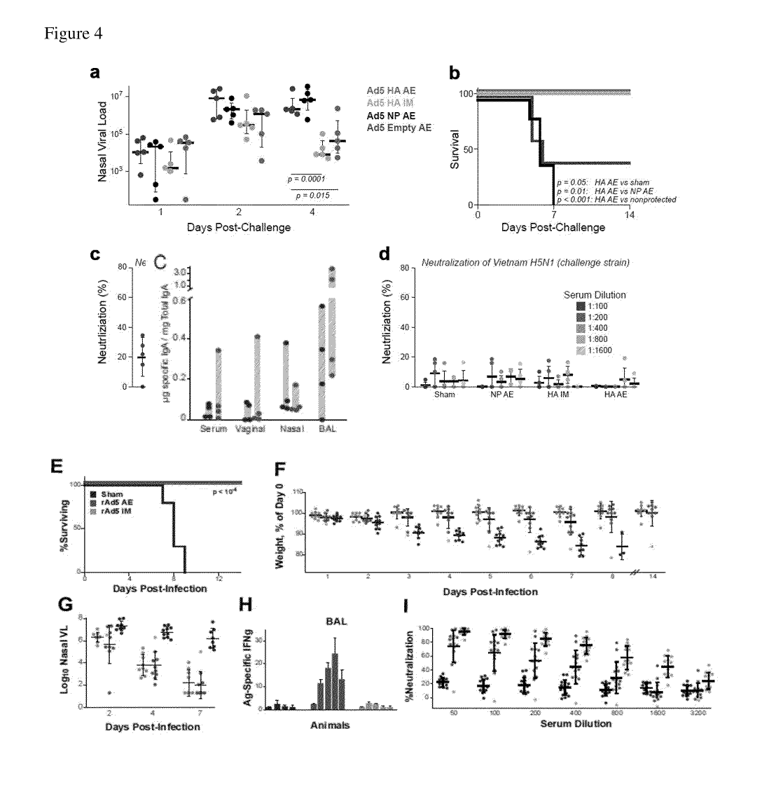

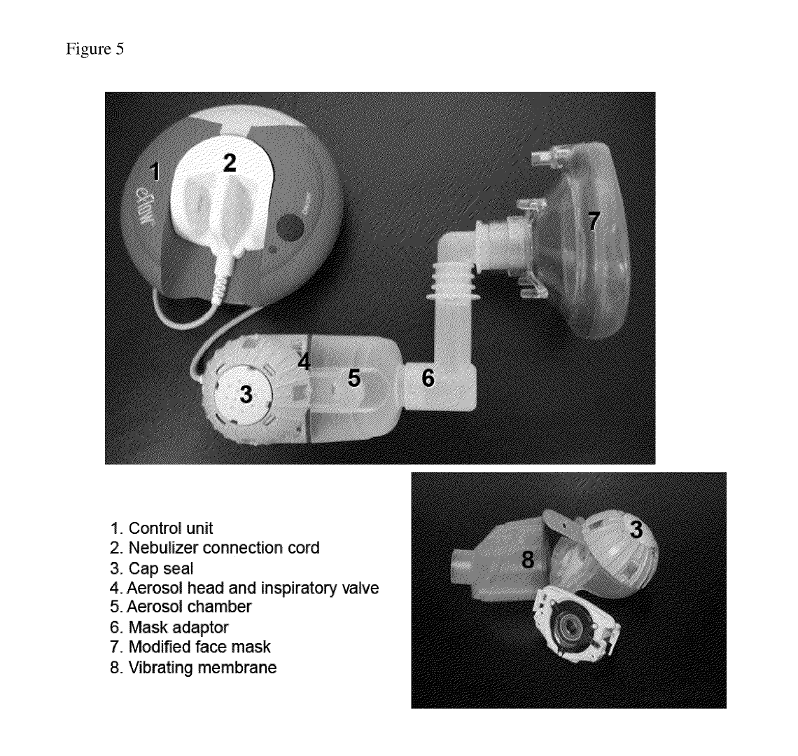

Immunization with sham or rAd5-NP by aerosol provided no protection. Animals immunized with rAd5-HA either by IM or by aerosol showed significant reductions in nasal viral loads by day 4 (FIGS. 4A and 4E). Similarly, as shown in FIG. 5, both vaccine groups were completely protected, with 100% survival (FIG. 4E) and nearly complete protection against weight loss (FIG. 4F)._The early control of viremia was also associated with a significantly improved survival. 3/5 sham-vaccinated and 5/5 NP-vaccinated animals were euthanized within 7 days due to illness (FIG. 4B and FIG. 4E). Only one of the two surviving sham-vaccinated animals eventually controlled viremia below 5×10⁴ on day 14. In contrast, 10/10 rAd5 HA aerosol- or IM-vaccinated animals survived to the end of the experiment (day 14); only two animals (one in each group) had not completely controlled virus by day 14, but still had viral loads well below 10⁴.

Cellular immunity in ferrets is difficult to quantify, as there are few monoclonal antibodies available for intracellular cytokine or ELISpot assays. In a separate pilot experiment, quantitative RT-PCR was used to verify that T cell responses were induced in the lungs of ferrets immunized using the same regimen used in this challenge study. The humoral response was quantified in the sera of the animals at the day of challenge (pre-immune sera were all negative). Vaccination elicited neutralizing titres to the vaccine strain (FIG. 4C, D), but only in the IM immunization regimen. No neutralization of the challenge strain was observed in any of the animals, suggesting that the principal mechanism of viral control (FIG. 4A, B) was either local mucosal antibody secretion, or a rapid anamnestic response to the challenge.

Additionally, the BAL-associated T cell responses in five animals from each group were quantified by quantitative RT-PCR (Svitek N. and von Messling V. (2007) Virology 326(2):404-10) to confirm that robust cellular immunity was induced by the aerosolized (but not IM) administration of the vaccine (FIG. 4H). Furthermore, vaccination by either route elicited neutralizing titres to the vaccine strain (FIG. 4I). Neutralization of the challenge strain was undetectable in sera from both vaccine groups at this early time point. Among the vaccine recipients, one animal with neutralizing tires below detection showed the highest nasal viral loads and significant weight loss, but was still able to control the virus and survive. Without being bound by any particular theory, it is believed that the mechanism of protection is most likely due to the humoral response. In any case, the immune responses elicited by the aerosolized vaccination provided a level of protection comparable to IM immunization.

The need for effective HIV, TB, and RSV vaccines have focused efforts on vaccine regimens that generate cellular and humoral immunity at mucosal sites. It is likely that highly effective cellular responses at these sites would significantly alter pathogenesis even after breakthrough infection. Protection from TB will require a strong cellular response in the lung prior to exposure, to circumvent the delay in the systemic T cell response at this site after infection. Similarly, protection against RSV will likely require a local, high-level Th1-biased cellular response in the lung soon after infection. As reported above, the utility of delivering immunogenic recombinant adenoviral (rAd) vectors directly to the lung was tested. Vaccine delivery to mucosal sites is typically achieved through introduction by intranasal or oral routes. To date, oral administration of rAd vectors has not elicited potent immune responses. Intranasal delivery of rAd5 vectors has raised safety concerns because the virus was found in the olfactory bulb of immunized animals. In contrast, aerosol delivery of vaccines has proven effective for measles vaccination. Several mucosal routes (nasal, oral and respiratory) have been tested: aerosol delivery generated good immunogenicity in the age ranges usually targeted for mass vaccination (9 months to 15 years) (Cutts et al., Biologicals 25 (3), 323 (1997)), suggesting that this could provide an alternative, cost-effective strategy to control this disease in developing countries. However, the physical-chemical characteristics of particles required to achieve this effect and the ability to extend this approach to other vaccines has been limited by a lack of understanding of the delivery requirements.

Here, a device was used that allowed definition of these properties. High density aerosols of rAd vaccines with mass median diameters (MMD) of 11.4 μm or 4.4 μm deposit in upper or total airway epithelia respectively (Martonen, Inhal Toxicol 13 (4), 307 (2001)). Distinct immunogenicity profiles were induced by the two different drop sizes in nonhuman primates. The smaller drop size aerosol was far more immunogenic, both for the transient systemic T cell response and for the systemic and mucosal humoral responses.

A number of features of the aerosolized rAd immunization facilitate its ability to serve as a mucosal vaccine. First, the immunization is largely silent with regard to the generation of anti-vector immune responses. Specifically, although humoral immunity to the antigens encoded by the vaccine insert is highly effective (and equivalent to IM immunization), the anti-adenoviral responses are nearly absent. In addition, the ability to stimulate both local cellular responses and systemic humoral responses is largely unaffected by pre-existing anti-vector antibodies. High rates of adenoviral seroprevalence are found in populations targeted for vaccination, especially in the developing world. This Ad5 seroprevalence has raised concerns that the utility of adenoviral vectors in humans may be limited (Barouch and Nabel Hum Gene Ther 16 (2), 149 (2005)). Although studies in humans and animals suggest that pre-existing immunity to adenoviral vectors may attenuate immune responses after IM immunization (Sumida et al., J Immunol 174 (11), 7179 (2005); Vogels et al., J Virol 77 (15), 8263 (2003)), mucosally-targeted delivery systems by aerosol delivery may overcome this potential difficulty. Furthermore, the aerosol approach allows consideration of homologous prime-boost regimens (where a boost could be given either by aerosol, or by IM to engage systemic responses), and the potential to administer the same vector for multiple different immunizations.

Immunization with aerosolized rAd generates durable, high magnitude, polyfunctional effector CD4 and CD8 T cell responses in the lung, ideal for regimens aimed at controlling TB, RSV, or influenza. This delivery route elicits humoral responses both at mucosal and systemic sites, including secretory IgA at mucosal sites. Because such delivery may elicit responses at distal sites, such as the rectum and vagina, it may serve as an adjunct to other immunization modalities that generate systemic T cell responses against sexually-transmitted pathogens, such as HIV.

In summary, aerosolized adenoviral delivery to the lung generates potent cellular and humoral immune responses with unique properties. This route of introduction elicited immune responses that fully protect against lethal influenza viral challenge. Importantly, this approach can be used repeatedly to generate humoral, Th1, and CD8 responses. The durable, high cellular responses induced in the lung may facilitate protection against TB, in which a major immunological deficit appears to be the long delay in trafficking of antigen-specific T cells to the lung, and may prove applicable to a range of challenging infectious diseases.

The results described above, were carried out using the following methods and materials.

### Aerosolization of Adenoviral Particles.

The Investigational e-Flow® Nebulizer System (PARI Pharma, Munich, Germany; FIG. 5) was used in compliance with the recommendations by the company and the Institutional Biosafety Office (IBC) at the NIH. The device is an electronic, portable nebulizer that utilizes a vibrating perforated membrane to generate aerosols of defined droplet sizes. Aerosol heads generating droplets with a mass median diameter (MMD) of 4.4 μm (±1.6 μm, geometric standard deviation) with the goal of delivering the immunogen deep into the lung, or alternatively aerosol heads generating droplets with a MMD of 11.4 μm (±1.8 μm) to target primarily the upper airways (Brand, P. et al., Intrapulmonary distribution of deposited particles. J Aerosol Med 12 (4), 275 (1999)). Geometric aerosol droplet size distribution using isotonic saline solution was determined by laser diffraction using a Malvern MasterSizerX (Malvern, Herrenberg, Germany). The delivery unit (mouthpiece and face mask) was changed with each immunization to avoid cross contamination between immunizations. Pediatric masks were customized to fit over the mouth and nares of either primates or ferrets. For immunization, 1 ml total volume of the adenoviral particles (concentration depending on dose) was aerosolized and delivered over 1 minute. Particles were detected inside the face mask but not a few inches away from the mask. Macaques were anesthetized for immunization; anesthetized animals had an inflated air cushion facemask placed over their mouth and nares and sealed with paper based tape. Ferrets were not anesthetized. After delivery of the entire reagent (1 ml total volume) over a 1 minute period, the mask was kept on the animal's face for an additional 3 minute to ensure complete inhalation. The mouth and nares were then washed with 70% ethanol followed by 3% hydrogen peroxide.

### Animals.

All animal studies were approved by the Animal Care and Use Committees of the Vaccine Research Center, NIAID, NIH, and of Bioqual, Inc. (Rockville, Md.). 37 colony-bred Indian-origin rhesus macaques and 20 ferrets housed and cared in accordance with local, state, federal and institute policies in an American Association for Accreditation of Laboratory Animal Care accredited facility were used in this study. All macaques were seronegative for SIV, SrV, and STLV-1. Blood and tissue specimens were collected prior to immunization, and at weekly or monthly time points following immunization. Ferrets were procured (Tripe F, Sayre, P A, USA) and tested seronegative for H5 and H1 influenza antibodies prior to study initiation. Body weights and temperature were collected prior to challenge and daily after influenza challenge for up to 8 days. Nasal washes for viral load were collected day 1, 2, 4, 6 and 7 post-infection. Remaining animals were euthanized 14 days after challenge. Lungs and trachea were collected and fixed from both moribund and euthanized animals.

### Evaluation of Animals.

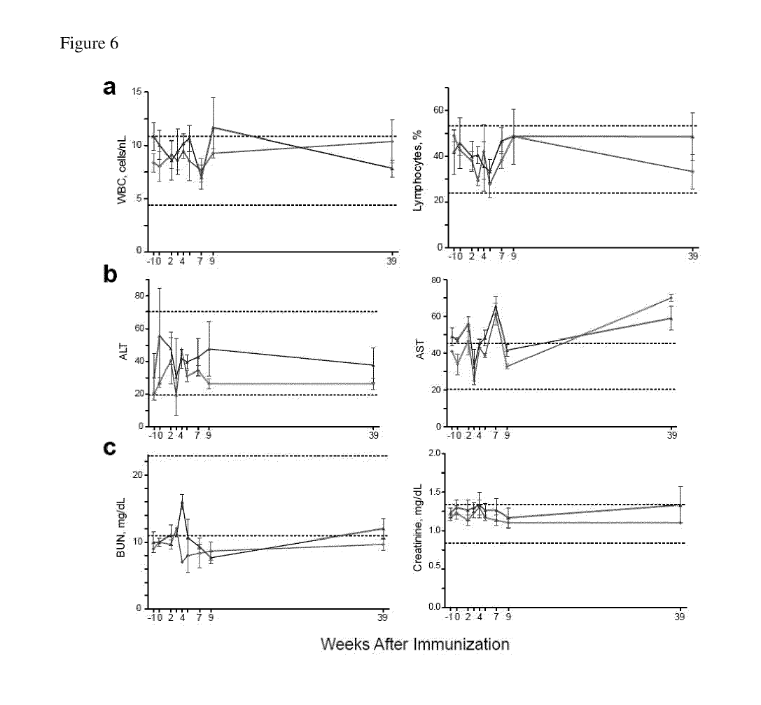

All animals tolerated the procedure extremely well and demonstrated no altered clinical signs upon daily monitoring by trained veterinary personnel. Periodic clinical pathology evaluation (CBC and clinical chemistry of the serum samples) did not reveal any changes affecting the health of the animals. Liver and kidney function tests were within normal limits for at least 40 weeks after immunization (FIGS. 6A-6C).

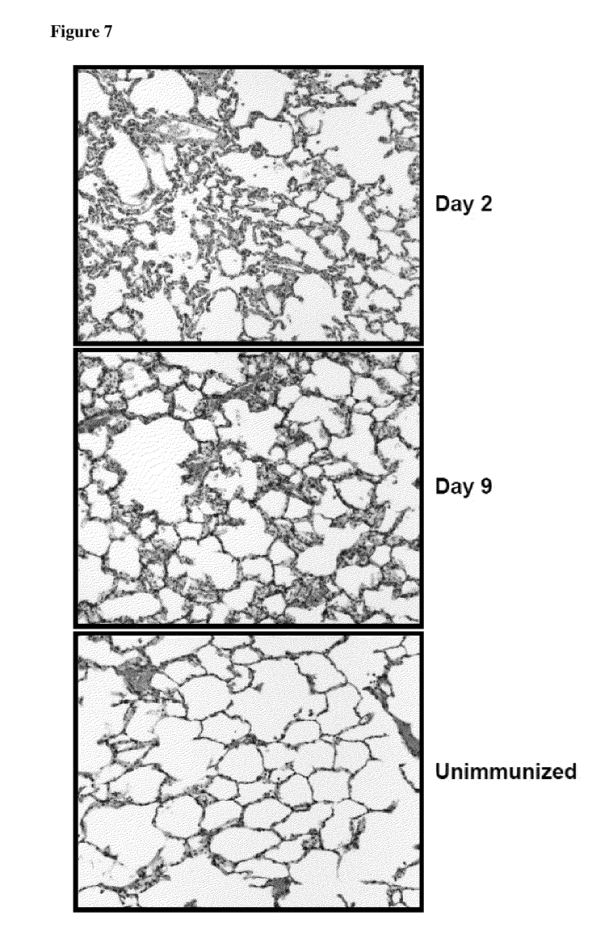

In order to evaluate the potential for primary lung immunization to potentially cause pathology, lung pathology was analyzed 2 or 9 days after immunization. There was minimal to absent pathology of the airways or the lung parenchyma (FIG. 7). Evaluation of the bronchial lymph nodes and the tonsils demonstrated moderate to marked lymphofollicular activation with expanding marginal zones. To date, more than 90 nonhuman primates have been immunized by this regimen and no negative effects have been observed.

### Cellular Immune Assays.

Assays on PBMC were performed on cryopreserved cells at a single assay time. Assays on bronchoalveolar lavage (BAL) cells were performed immediately upon isolation, following filtration through a 70 μm cell strainer. All antibodies were purchased from Becton Dickinson, Inc. (San Jose, Calif.), either conjugated, or unconjugated and derivatized. All reagents were qualified and titrated using rhesus macaque PBMC. HIV Env, SIV Env and SIV Gag-Pol specific responses were determined using overlapping peptides as previously described (Betts, et al., J Virol 75 (24), 11983 (2001); Maecker, H. T. et al., J Immunol Methods 255 (1-2), 27 (2001); Mattapallil, J. J. et al., J Exp Med 203 (6), 1533 (2006)). Control cultures were set up for each sample with anti-CD28 and anti-CD49d in the absence of peptides. Following stimulation, cells were labeled with cell surface markers (CD4-Alexa700APC, CD8-QDot655, CD45RA-TRPE, and CD95-APC) and ViViD (to discriminate live/dead cells) (Perfetto et al., J Immunol Methods 313 (1-2), 199 (2006)). After fixing, cells were permeabilized and labeled with IFNγ-FITC, TNFα-Cy7PE, IL-2-PE, and CD3-Cy7APC. Labeled cells were analyzed using a modified Becton Dickinson LSR II. For ELISpot assays, PBMC (2×10⁵ cells) were incubated with final concentration of 2 μg/ml peptide for 18-20 hrs at 37° C. in a human IFN-γ ELISpot plate and processed as per kit instructions (Becton Dickinson, Inc.). Briefly, the plate was washed 4 times with washing buffer followed by 2 washes with dH₂O. 2 μg/ml of detector antibody (U-Cytech, Utrecht, Netherlands) was added and incubated for 2 hours at room temperature. Plates were washed 3 times with wash buffer and then incubated for 1 hour with 100 μl avidin-HRP. The plates were developed with AEC Substrate and read on a CTL reader after drying overnight.

### Humoral Immune Assays.

These assays were performed by ELISA as previously described (Letvin, et al., J Virol 81 (22), 12368 (2007)). Briefly, specific IgA or IgG antibodies to HIV env, SIV env, or SIV Gag/Pol antigens were measured using microtiter plates coated respectively with 100 ng/well HIV Clade A rgp140, 100 ng/well SIVmac251 rgp130 (ImmunoDiagnostics, Woburn, Mass.) or 125 ng total protein/well of SIVmac251 viral lysate, an amount which lacks detectable envelope protein (Advanced Biotechnologies Inc, Columbia, Md.). Total IgA or IgG was measured using plates coated with 50 ng/well goat anti-monkey IgA (Rockland, Gilbertsville, Pa.) or 60 ng total protein/well of goat whole IgG fraction to monkey IgG (MP BioMedicals, Solon, Ohio). The standards were pooled macaque serum containing previously calibrated amounts of the relevant antibody or immunoglobulin (Letvin, et al., J Virol 81 (22), 12368 (2007)). Secondary reagents were 25 ng/ml biotinylated goat anti-monkey IgA (OpenBiosystems, Huntsville, Ala.) or 200 ng/ml anti-human IgG (Southern Biotech, Birmingham, Ala.) and 0.5 μg/ml avidin-labeled peroxidase (Sigma). Plates for antigen-specific ELISAs were developed with tetramethylbenzidine substrate (Sigma) for 30 minutes, stopped with 2N sulfuric acid, then read at 450 nm in a SpectraMax M5 plate reader (Molecular Devices, Sunnyvale, Calif.). Absorbance values in total IgA or IgG ELISAs were recorded at 414 nm after treatment with 2,2-azinobis(3-ethylbenzthiazolinesulfonic acid). The concentration of antigen-specific IgA or IgG antibody measured in each sample was divided by the concentration of total IgA or IgG to obtain the “specific activity”.

### Histology

4% formaldehyde fixed lung tissue was sectioned at 5 μm, stained with hematoxylin and eosin using standard methods and evaluated by a board certified pathologist.

### Viral Neutralization Assays

Adenoviral neutralizing titres were performed by the NVITAL laboratory (Rockville, Md.) based on a published assay (Sprangers, et al., J Clin Microbiol 41 (11), 5046 (2003)). Influenza virus neutralization was performed as described (Yang, et al., Science 317 (5839), 825 (2007)).

### Statistics.

Comparisons of distributions were by the Students T test, using either JMP (SAS Institute, Cary, N.C.) or SPICE (VRC, NIH).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of inducing a pathogen-specific immune response in a subject, the method comprising selectively administering to the respiratory tract of a subject an aerosol genetic vaccine comprising an effective amount of an expression vector comprising a polynucleotide encoding a pathogen polypeptide and a pharmaceutically acceptable excipient.

2. The method of claim 1, wherein the method treats or prevents a pathogen infection in a the subject.

3. The method of claim 1, wherein the method induces a cellular immune response in a subject's lung.

4. The method of claim 3, wherein the method further induces a humoral immune response in lung, peripheral blood, and mucosal sites.

5. The method of claim 1, wherein the method induces a systemic humoral response in a subject.

6. The method of claim 1, wherein the method induces a pathogen-specific humoral and/or cellular immune response in a subject having pre-existing anti-adenoviral immunity.

7. The method of claim 1, wherein the pathogen polypeptide is a bacterial, viral, fungal, or yeast polypeptide.

8. The method of claim 7, wherein the viral polypeptide is an influenza, respiratory syncytial virus (RSV), SIV or HIV polypeptide.

9. The method of claim 8, wherein the viral polypeptide is influenza hemagglutinin (HA), influenza nucleoprotein, HIV Env, SIV Env, Gag, or Pol, and the bacterial polypeptide is tuberculosis 10.4 or 85A/B antigens.

10. The method of claim 1, wherein the method induces a protective immune response against a mucosal pathogen.

11. The method of claim 1, wherein the composition comprises particles having a mass median diameter between about 1 and 15 μm, about 3-5 μm or 9-12 μm, or about 11.4 μm or 4.4 μm.

12. The method of claim 1, wherein the immunity persisted for at least about 3, 6, 9 or 12 months.

13. The method of claim 1, wherein the method generates a negligible anti-vector immune response.

14. The method of claim 1, wherein the method induces an antibody response at mucosal surfaces.

15. A method of selectively administering to the upper airway of a subject an aerosol genetic vaccine comprising an effective amount of an expression vector comprising a polynucleotide encoding a pathogen polypeptide and a pharmaceutically acceptable excipient.

16. The method of claim 15, wherein particles of said vaccine have a mass median diameters of about 11.4 μm.

17. A method for inducing protective immunity in a subject against tuberculosis, the method comprising administering to the subject an aerosol genetic vaccine comprising an adenoviral vector comprising a polynucleotide encoding tuberculosis antigens 85A/B or 10.4.

18. An immunogenic composition comprising an effective amount of an expression vector comprising a polynucleotide encoding a pathogen polypeptide and a pharmaceutically acceptable excipient, wherein the immunogenic composition is formulated for aerosol delivery to the lung.

19. An immunogenic composition comprising an adenoviral expression vector comprising a pathogen polynucleotide encoding a polypeptide selected from the group consisting of TB 85A/B or 10.4 antigens, influenza hemagglutinin or nucleoprotein, HIV or SIV Gag-Pol and HIV or SIV Env, wherein the pathogen polynucleotide is positioned for expression in a mammalian cell and a pharmaceutically acceptable excipient, wherein the immunogenic composition is formulated for aerosol delivery to lung tissue.

20. The immunogenic composition of claim 19, wherein the composition comprises particles having a mass median diameter between about 1 and 15 μm, about 3-5 μm or 9-12 μm, or about 11.4 μm or 4.4 μm.

21. A genetic vaccine in an amount sufficient to induce a protective immune response in a subject, the vaccine comprising an adenoviral expression vector comprising a pathogen polynucleotide encoding a polypeptide selected from the group consisting of TB 85A/B or 10.4 antigens, influenza hemagglutinin or nucleoprotein, HIV or SIV Gag, Pol or Env, having a mass median diameter between about 1 and 15 μm and a pharmaceutically acceptable excipient, wherein the vaccine is formulated for aerosol delivery to the lung.

22. A pharmaceutical pack comprising an effective amount of an immunogenic composition of claim 19, a device for aerosol delivery of said composition, and written instructions for use of the pack for the treatment or prevention of a pathogen infection.

23. A device for dispersing a genetic vaccine into particles and delivering a dose of said particles to a subject, the device comprising an immunogenic composition of claim 19.

24. The device of claim 23, wherein the device is a nebulizer, metered dose inhaler or dry powder inhaler.

25. The device of claim 23, wherein the device selectively delivers the vaccine to total lung tissue.

26. A method for production of an aerosol genetic vaccine for the treatment or prevention of a pathogen infection, the method comprising
(a) providing an effective amount of an immunogenic composition comprising an effective amount of an expression vector comprising a polynucleotide encoding a pathogen polypeptide and a pharmaceutically acceptable excipient; and
(b) dispersing the composition into particles having a median mass median diameter of between 1 and 15 μm.

27. The method of claim 1, wherein the selective administration targets the upper respiratory tract, central airways, lung surface, alveoli, or deep lung.

28. The method of claim 27, wherein the method induces a stable lung T cell response.


SEQUENCE LISTING (19 sequences)

SEQ ID NO: 1 - DNA, 18 bp, Artificial Sequence
  Description of Artificial Sequence Synthetic primer
agaggaacacattccccc

SEQ ID NO: 2 - DNA, 32 bp, Artificial Sequence
  Description of Artificial Sequence Synthetic primer
ggggagaaaggactgtgtattctgtcaaatgg

SEQ ID NO: 3 - DNA, 35 bp, Artificial Sequence
  Description of Artificial Sequence Synthetic primer
tttgacagaatacacagtcctttctccccggctgg

SEQ ID NO: 4 - DNA, 33 bp, Artificial Sequence
  Description of Artificial Sequence Synthetic primer
acaaaatacgagaatgactacgtccggcgttcc

SEQ ID NO: 5 - DNA, 30 bp, Artificial Sequence
  Description of Artificial Sequence Synthetic primer
ggacgtagtcattctcgtattttgtatagc

SEQ ID NO: 6 - DNA, 18 bp, Artificial Sequence
  Description of Artificial Sequence Synthetic primer
tcaccaacacagtggggg

SEQ ID NO: 7 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 8 - DNA, 1364 bp, Mycobacterium tuberculosis
  modified_base (1275)..(1276): a, c, g, t, unknown or other
cgacacatgcccagacactgcggaaatgccaccttcaggccgtcgcgtcggtcccgaattggccgtgaacgaccgccggataagggtttcggcggtgcgcttgatgcgggtggacgcccgaagttgtggttgactacacgagcactgccgggcccagcgcctgcagtctgacctaattcaggatgcgcccaaacatgcatggatgcgttgagatgaggatgagggaagcaagaatgcagcttgttgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgcctagctccgaacagacacaacatctagcnncggtgacccttgtggnncanatgtttcctaaatcccgtccctagctcccgcngcnnccgtgtggttagctacctgacnncatgggttt

SEQ ID NO: 9 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 10 - DNA, 978 bp, Mycobacterium tuberculosis
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctga

SEQ ID NO: 11 - protein, 96 aa, Mycobacterium tuberculosis
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 12 - DNA, 291 bp, Mycobacterium tuberculosis
atgtcgcaaatcatgtacaactaccccgcgatgttgggtcacgccggggatatggccggatatgccggcacgctgcagagcttgggtgccgagatcgccgtggagcaggccgcgttgcagagtgcgtggcagggcgataccgggatcacgtatcaggcgtggcaggcacagtggaaccaggccatggaagatttggtgcgggcctatcatgcgatgtccagcacccatgaagccaacaccatggcgatgatggcccgcgacacggccgaagccgccaaatggggcggctag

SEQ ID NO: 13 - protein, 552 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNNEEEQTRLYQNPTTYISIGTSTLNQRLVPKIATRSKVNGQSGRMEFFWTILKPNDAINFESNGNFIAPEYAYKIVKKGDSAIMKSELEYSNCNTKCQTPMGAINSSMPFHNIHPLTIGECPKYVKSSRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMIAGLSLWMCSNGSLQCRICI

SEQ ID NO: 14 - protein, 500 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 15 - protein, 844 aa, Human immunodeficiency virus 1
VRGIQTSWQNLWRWGTMILGMLVIYSAAENLWVAVYYGVPVWKDAETTLFCASDAKAYDTEVHNVWETHACVPTDPNPQEIHLENVTEDFNMWRNNMVEQMHTDIISLWDQSLKPCVKLTPLCVTLDCNATASNVTNEMRNCSFNITTELKDKKQQVYSLFYKLDVVQINEKNETDKYRLINCNTSAITQACPKVSFEPIPIHYCAPAGFAILKCKDTEFNGTGPCKNVSTVQCTHGIRPVISTQLLLNGSLAEEGIQIRSENITNNAKTIIVQLDKAVKINCTRPNNNTRKGVRIGPGQAFYATGGIIGDIRQAHCHVSRAKWNDTLRGVAKKLREHFKNKTIIFEKSSGGDIEITTHSFICGGEFFYCNTSGLFNSTWESNSTESNNTTSNDTITLTCRIKQIINMWQKVGQAMYPPPIQGVIRCESNITGLLLTRDGGNNSTNEIFRPGGGNMRDNWRSELYKYKVVKIEPLGVAPSRAKRRVVEREKRAVGIGAVFLGFLGAAGSTMGAASITLTAQARQLLSGIVQQQSNLLRAIEAQQHMLKLTVWGIKQLQARVLAVERYLKDQQLLGIWGCSGKLICTTNVPWNSSWSNKSMNEIWDNMTWLQWDKEISNYTQIIYNLIEESQNQQEKNEQDLLALDKWASLWNWFDISRWLWYIKIFIMIVGGLIGLRIVFAVLSVINRVRQGYSPLSFQIRTPNPKEPDRLGRIDGEGGEQDRDRSIRLVSGFLALAWDDLRSLCLFSYHRLRDFISIAARTVELLGHSSLKGLRLGWEGLKYLWNLLLYWGRELKTSAVNLVDTIAIAVAGRTDRVIEVGQRIFRAILNIPRRIRQGLERGLL

SEQ ID NO: 16 - protein, 1003 aa, Human immunodeficiency virus 1
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDGLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 17 - DNA, 36146 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagtaacggccgccagtgtgctggaattcgcccttgatttaaacgaattctcaggccacctcgcgggcctcgccggtgtcctccatgtggctgctgctgtccacctccttgccgccgccgtagtccttgatgatcttggccttgcggcggggcaccaccttgatgtcggtgcccaccttcaggatcacggcgccctcgcccttccacagcagctcgccggggcccttccacagctggtcgcggccctcgcggtagtacacgcggaagttcttgaacttgctgttcttgctctgctggaactggatctcctgctcggtggtgatcatgttgatcaggcgctcggcgggcgtcatgtcgccgatgccgccgcggcgcttgaagttcatgcagtgcacggccatcagcacgatggtctccacgctgttggcctgctcgcggatgcggtcgatctggttcttcaggtggtggttcatggcctccaccacgccctggctctgcgggttgtagggcacgccgaaggtgtgctcgatgccggcccaccaggccaccatcttcacctcctggctggcgaagttggcgccgttgtcggtgtgcaggtgggtgatgggccagcggccggccagcttcagcaggaacagggcggtctggcggccggtctcctgcgggatcacctcggcctcgatgaagccgctggccacgtgcacggccacgatgatgatcttgccctccaggtgggtgcaggccatctgccaggtgcccaggtcgctgttggcctggccgtggatggcctcgcccttctggtggcacttgtcgcaggtgtccacgatctggcgggccacgatgcggggcaggccgaacttgaacaccagctccttcacgttgctgtggtacttgtcgtgctcctcctgggcgggctcgatcttctccaggaacagcacctggcggatgccctggctcaccaggtggtcgatctcctggttgccgccgatgcccttgtgggcgggcacccaggccacgtagatctcgctcttcttgatcatctcctcgatgatctggttcaccaggcggctctcgctctcggtggggcagccggtgatgatgcccatcacgtactggctgtccacgatgatgttggccttggggccgctgtcggtcagggccatcaggaaggcctcgagctcggcctgctggttggtggtctgctccagcaccttcaccttgtccttgccgcggtcggtgatgtagccggccttgccctccttgctctgcttgttgcagctgccgtcggtgtagtaggtctcctcgccctcgatggggtccttcaccaggttgaacaccaggcgcaccaggggaggggtgctgatgaagtcccactcgggtatccaggtcacctgccagtagtcggtccaccactgctcccacacgtccttctccacgggcaggtggaacttgggcacctggccccagatcacgatggcctccttgccgatcttctggatcacgtgggccagcaggcgcacgccgttggtgtgggtgttcttgatcttggcgaacttgcccaccttcaggatcttgtcctcctggtggatcttgtagctccactggttgtcctggctcttgatcacggtggcctccaggggcttgccctcctggtagtagcagccctcctgctcctggctcaggatgatcttgttctcctcgtactcggcctcggccatctcggtccactgcacctcctcggtcagggtcatcttgccgcggatcaggcggcacaggtgcttggtcttgatgccggggtagatctgggcggcccagttcagcacgcccaccagcttctggatgtcgttcacggtccaggtctcgcgctggggcagctcgatcttctgcagcttccacttggtgggccacagctcgtagcccatccactggaagggtgggtccttctggaacttctcctcgggcgtgctgaagccgatgctgttcagcagctccttgctctgcagcaccacgcggtcgtgctccaggtcggtgcggtcgctggcgatcaggatgtcatgcatgtactgcaccagggtcacgtcggggttggccttgcggaagggctccagcacgtggcgcatggtgtactggaagatggcggggctgcccttccagccctggggcagcaccttgtagatgtagcgcttgccgggctcggcgttgttcacgctgggcagggtgaaggcggtgtactggcggaactcctcgtccagggggatgctgaagtaggcgtcgccgatgtccagcacggtgatgcgcttgcgcttggccaggccggcggggtgcgggatgcccagctgcacctcggtgaagtcctgggtcacgcggttcagctcgcggaagtcgatcagcatgcgccacttgttcttgtccttcttcttgatggcgaaggtgggcgtgttgtaggggttggtgggtggggcctcctccagctggccgtccttctccatcttctcgcagatctcgcgcagggccacgatcttctccttgctcaggggccactgcttcagcttggggccgtccttgccgggcttcagggccaccttcacgggctccaccttggcgatggggaagttcaggctcatgcccagggcggtcagcaggttgcggccgaagatgttgatgggcgtgtcgccggtcatgatggtgcccttgatgcgcttgcccagcacctcgatctccacgttcttgtactctccggtgttgatgaagccgccgatgccgcccacgatcttcggggtgtagtgcgggcccagctcgatgccggtcacgatgctgtcgtcggcgccggtgtctagaagcacctccacgggctggccctcgatcgatgcagtgactactggtctcctccaaagagagaattgaggtgcagcaaatcctctgtcacctccttgtaaggcttctctctgctttctctctgcttttctctctgctgcttgcccaactgcatgtagttctttagcagatccacagctgggtcctctgggggagcagttggcatcagcccctgatgcacttgagccatggggaaattgcggggcttctttccccatggaccaaggcctacccgcctggcggtccgggcacttggccatcacgtggtccatcttgccgcacttccagcagccctggcggcgcggcgcgcggcactggcgggcgctgtggccctccttgccgcagttccagcacttgatgggcttgcgcgggccgcgctgctgggcggccgcgaacgggatgggcacgggcgccagggcctccttcagggcctcggccatcaggcgggccttctggcccgggccgcccacgccctggcaggcggtcagcatctcctccagggtcgggttcacgcccaggcccttcagcaccagcttgcagtccgggttggcgttctggatcagcagggtctgggtcatccagttcttcacggcggcgtcggtctgctcggcgcgcaggctcttgtagaagcggtccacgtagctctggaagggctccttcgggccctgcttcacgtccaggatgttggtcgggttgtacatgcgcacgcacttctgcaggcccagctgtatccagcggcggtagatgttgcccaccgggatcgggttctgctggcggtacatccactggatctgctcgtccacgctgctggtggtgccggcgatgtcgctgccgctgggctcgcgcagctggccctgctgcggcgcgggctgcgggtgctgcaggtcccagtcggcggcctcctcgttgatgatgtcgcggatgatctgcatggcggcctggtggtcgcccacgcagttcagcatctggttgatgtcgtagggcgtgcagccctcgctcagggcctggaagccgggcaccacctcggcgccgaacttcttctcctcgatcagcttcacccaggcgttcagggtgcgcgggctcagcggcaggtgcacgtagttgccgccgatctgctgcaccgggtagttgccgccgcgtccgctgctgggcgcggtcgggcggctggtcttgggcatggtctcggtggtgccggtctccaccaccaggtggcgctgcacgatctgcttggcctcctcggtgtgcttcaccttctcctcggcgtggatgcaccagatcacgcacacggtgttgtacaggctcttcaggttctcgctgccggtgggcaccagcggcgccagcacgctcaggatcttctggcagccctccttgttctccagcaggctctcggccaggccgaagcggtccagctcgttggcggcccacaccacgtgcttcagcatgtacttcttcttgccgttcgggcgcaggcggatcttctccagctcgtcggccttcttgccgctcagcacgctgttgcgcacgcccatggtggcggcgtcgaatctgatcacacgtgtcgaaaatggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgcgatccggcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatgcgttcgacctttctcttcttttttgggcccatgatggcagatccgtatagtgagtcgtattagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagatgcatgctcgagcggccgccagtgtgatggatatctgcagaattccagcacactggcggccgttactagtggatccgagctcggtacccggccgttataacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 18 - DNA, 4388 bp, Artificial Sequence
  Description of Artificial Sequence Synthetic polynucleotide
gccgccaccatgggcgtgcgcaacagcgtgctgagcggcaagaaggccgacgagctggagaagatccgcctgcgcccgaacggcaagaagaagtacatgctgaagcacgtggtgtgggccgccaacgagctggaccgcttcggcctggccgagagcctgctggagaacaaggagggctgccagaagatcctgagcgtgctggcgccgctggtgcccaccggcagcgagaacctgaagagcctgtacaacaccgtgtgcgtgatctggtgcatccacgccgaggagaaggtgaagcacaccgaggaggccaagcagatcgtgcagcgccacctggtggtggagaccggcaccaccgagaccatgcccaagaccagccgcccgaccgcgcccagcagcggacgcggcggcaactacccggtgcagcagatcggcggcaactacgtgcacctgccgctgagcccgcgcaccctgaacgcctgggtgaagctgatcgaggagaagaagttcggcgccgaggtggtgcccggcttccaggccctgagcgagggctgcacgccctacgacatcaaccagatgctgaactgcgtgggcgaccaccaggccgccatgcagatcatccgcgacatcatcaacgaggaggccgccgactgggacctgcagcacccgcagcccgcgccgcagcagggccagctgcgcgagcccagcggcagcgacatcgccggcaccaccagcagcgtggacgagcagatccagtggatgtaccgccagcagaacccgatcccggtgggcaacatctaccgccgctggatacagctgggcctgcagaagtgcgtgcgcatgtacaacccgaccaacatcctggacgtgaagcagggcccgaaggagcccttccagagctacgtggaccgcttctacaagagcctgcgcgccgagcagaccgacgccgccgtgaagaactggatgacccagaccctgctgatccagaacgccaacccggactgcaagctggtgctgaagggcctgggcgtgaacccgaccctggaggagatgctgaccgcctgccagggcgtgggcggcccgggccagaaggcccgcctgatggccgaggccctgaaggaggccctggcgcccgtgcccatcccgttcgcggccgcccagcagcgcggcccgcgcaagcccatcaagtgctggaactgcggcaaggagggccacagcgcccgccagtgccgcgcgccgcgccgccagggctgctggaagtgcggcaagatggaccacgtgatggccaagtgcccggaccgccaggcgggtaggccttggtccatggggaaagaagccgcgcaatttcccgatggctcaagtgcatcaggggctgatgccaactgctccgccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgcatcgatcgagggccagcccgtggaggtgcttctagacaccggcgccgacgacagcatcgtgaccggcatcgagctgggcccgcactacaccccgaagatcgtgggcggcatcggcggcttcatcaacaccggagagtacaagaacgtggagatcgaggtgctgggcaagcgcatcaagggcaccatcatgaccggcgacacgcccatcaacatcttcggccgcaacctgctgaccgccctgggcatgagcctgaacttccccatcgccaaggtggagcccgtgaaggtggccctgaagcccggcaaggacggccccaagctgaagcagtggcccctgagcaaggagaagatcgtggccctgcgcgagatctgcgagaagatggagaaggacggccagctggaggaggccccacccaccaacccctacaacacgcccaccttcgccatcaagaagaaggacaagaacaagtggcgcatgctgatcgacttccgcgagctgaaccgcgtgacccaggacttcaccgaggtgcagctgggcatcccgcaccccgccggcctggccaagcgcaagcgcatcaccgtgctggacatcggcgacgcctacttcagcatccccctggacgaggagttccgccagtacaccgccttcaccctgcccagcgtgaacaacgccgagcccggcaagcgctacatctacaaggtgctgccccagggctggaagggcagccccgccatcttccagtacaccatgcgccacgtgctggagcccttccgcaaggccaaccccgacgtgaccctggtgcagtacatgcatgacatcctgatcgccagcgaccgcaccgacctggagcacgaccgcgtggtgctgcagagcaaggagctgctgaacagcatcggcttcagcacgcccgaggagaagttccagaaggacccacccttccagtggatgggctacgagctgtggcccaccaagtggaagctgcagaagatcgagctgccccagcgcgagacctggaccgtgaacgacatccagaagctggtgggcgtgctgaactgggccgcccagatctaccccggcatcaagaccaagcacctgtgccgcctgatccgcggcaagatgaccctgaccgaggaggtgcagtggaccgagatggccgaggccgagtacgaggagaacaagatcatcctgagccaggagcaggagggctgctactaccaggagggcaagcccctggaggccaccgtgatcaagagccaggacaaccagtggagctacaagatccaccaggaggacaagatcctgaaggtgggcaagttcgccaagatcaagaacacccacaccaacggcgtgcgcctgctggcccacgtgatccagaagatcggcaaggaggccatcgtgatctggggccaggtgcccaagttccacctgcccgtggagaaggacgtgtgggagcagtggtggaccgactactggcaggtgacctggatacccgagtgggacttcatcagcacccctcccctggtgcgcctggtgttcaacctggtgaaggaccccatcgagggcgaggagacctactacaccgacggcagctgcaacaagcagagcaaggagggcaaggccggctacatcaccgaccgcggcaaggacaaggtgaaggtgctggagcagaccaccaaccagcaggccgagctcgaggccttcctgatggccctgaccgacagcggccccaaggccaacatcatcgtggacagccagtacgtgatgggcatcatcaccggctgccccaccgagagcgagagccgcctggtgaaccagatcatcgaggagatgatcaagaagagcgagatctacgtggcctgggtgcccgcccacaagggcatcggcggcaaccaggagatcgaccacctggtgagccagggcatccgccaggtgctgttcctggagaagatcgagcccgcccaggaggagcacgacaagtaccacagcaacgtgaaggagctggtgttcaagttcggcctgccccgcatcgtggcccgccagatcgtggacacctgcgacaagtgccaccagaagggcgaggccatccacggccaggccaacagcgacctgggcacctggcagatggcctgcacccacctggagggcaagatcatcatcgtggccgtgcacgtggccagcggcttcatcgaggccgaggtgatcccgcaggagaccggccgccagaccgccctgttcctgctgaagctggccggccgctggcccatcacccacctgcacaccgacaacggcgccaacttcgccagccaggaggtgaagatggtggcctggtgggccggcatcgagcacaccttcggcgtgccctacaacccgcagagccagggcgtggtggaggccatgaaccaccacctgaagaaccagatcgaccgcatccgcgagcaggccaacagcgtggagaccatcgtgctgatggccgtgcactgcatgaacttcaagcgccgcggcggcatcggcgacatgacgcccgccgagcgcctgatcaacatgatcaccaccgagcaggagatccagttccagcagagcaagaacagcaagttcaagaacttccgcgtgtactaccgcgagggccgcgaccagctgtggaagggccccggcgagctgctgtggaagggcgagggcgccgtgatcctgaaggtgggcaccgacatcaaggtggtgccccgccgcaaggccaagatcatcaaggactacggcggcggcaaggaggtggacagcagcagccacatggaggacaccggcgaggcccgcgaggtggcctgagaattcgtttaaacacgcgtgtatac

SEQ ID NO: 19 - DNA, 1964 bp, Artificial Sequence
  Description of Artificial Sequence Synthetic polynucleotide
gaattcgtttaaacacgcgtgtatacgccgccaccatgggctgcctgggcaaccagctgctgatcgccatcctgctgctgagcgtgtacggcatctactgcaccctgtacgtgaccgtgttctacggcgtgcccgcctggcgcaacgccaccatcccgctgttctgcgccaccaagaaccgcgacacctggggcaccacccagtgcctgcccgacaacggcgactacagcgaggtggccctgaacgtgaccgagagcttcgacgcctggaacaacaccgtgaccgagcaggccatcgaggacgtgtggcagctgttcgagaccagcatcaagccctgcgtgaagctgagcccgctgtgcatcaccatgcgctgcaacaagagcgagaccgaccgctggggcctgaccaagagcatcaccaccaccgccagcaccaccagcaccaccgccagcgccaaggtggacatggtgaacgagaccagcagctgcatcgcccaggacaactgcaccggcctggagcaggagcagatgatcagctgcaagttcaacatgaccggcctgaagcgcgacaagaagaaggagtacaacgagacctggtacagcgccgacctggtgtgcgagcagggcaacaacaccggcaacgagagccgctgctacatgaaccactgcaacaccagcgtgatccaggagagctgcgacaagcactactgggacgccatccgcttccgctactgcgcgccacccggctacgccctgctgcgctgcaacgacaccaactacagcggcttcatgcccaagtgcagcaaggtggtggtgagcagctgcacccgcatgatggagacccagaccagcacctggttcggcttcaacggcactcgagccgagaaccgcacctacatctactggcacggccgcgacaaccgcaccatcatcagcctgaacaagtactacaacctgaccatgaagtgccgccggcccggcaacaagaccgtgctgcccgtgaccatcatgagcggcctggtgttccacagccagcccatcaacgaccgcccgaagcaggcctggtgctggttcggcggcaagtggaaggacgccatcaaggaggtgaagcagaccatcgtgaagcacccacgctacaccggcaccaacaacaccgacaagatcaacctgaccgcgcccggcggcggcgacccggaggtgaccttcatgtggaccaactgccgcggcgagttcctgtactgcaagatgaactggttcctgaactgggtggaggaccgcaacaccgccaaccagaagcccaaggagcagcacaagcgcaactacgtgccctgccacatccgccagatcatcaacacctggcacaaggtgggcaagaacgtgtacctgccaccgcgcgagggcgacctgacctgcaacagcaccgtgaccagcctgatcgccaacatcgactggatcgacggcaaccagaccaacatcaccatgagcgccgaggtggccgagctgtatcgattggagctgggcgactacaagctggtggagatcacgcccatcggcctggcgcccaccgacgtgaagcgctcgctgaccctgaccgcccagagccgcaccctgctggccggcatcgtgcagcagcagcagcagctgctggacgtggtgaagcgccagcaggagctgctgcgcctgaccgtgtggggcaccaagaacctgcagacccgcgtgaccgccatcgagaagtacctgaaggaccaggcccagctgaacgcgtccctgacgcccaagtggaacaacgagacctggcaggagtgggagcgcaaggtggacttcctggaggagaatatcaccgccctgctggaggaggcccagatccagcaggagaagaacatgtacgagctgcagaagctgaacagctgggacgtgttcggcaactggttcgacctggctagctggatcaagtacatccagtga